SEQUENCE LISTING (151 sequences)

SEQ ID NO: 1 - protein, 5 aa, Artificial Sequence
  Adenovirus protease substrate consensus
XXGXG

SEQ ID NO: 2 - protein, 5 aa, Artificial Sequence
  Adenovirus protease substrate consensus
XXXXG

SEQ ID NO: 3 - protein, 5 aa, Artificial Sequence
  Alphavirus protease substrate consensus
XGXXX

SEQ ID NO: 4 - protein, 7 aa, Artificial Sequence
  Rubella virus protease substrate consensus
SRGGGTC

SEQ ID NO: 5 - protein, 7 aa, Artificial Sequence
  HIV protease substrate consensus
XXXXPXX

SEQ ID NO: 6 - protein, 7 aa, Artificial Sequence
  HTLV protease substrate consensus
XXXXVXQ

SEQ ID NO: 7 - protein, 10 aa, Artificial Sequence
  HTLV protease substrate consensus
KVKVXVVQPK

SEQ ID NO: 8 - protein, 7 aa, Artificial Sequence
  HTLV protease substrate consensus
PPXXLPI

SEQ ID NO: 9 - protein, 3 aa, Artificial Sequence
  Arteri virus protease substrate consensus
XEX

SEQ ID NO: 10 - protein, 8 aa, Artificial Sequence
  Coronavirus 3C protease substrate consensus
XXLQXGXX

SEQ ID NO: 11 - protein, 6 aa, Artificial Sequence
  SARS corona virus PLpro protease substrate consensus
XXGGXX

SEQ ID NO: 12 - protein, 7 aa, Artificial Sequence
  SARS corona virus 3CL protease substrate consensus
XXLQSGF

SEQ ID NO: 13 - protein, 4 aa, Artificial Sequence
  Torovirus protease substrate consensus
XXQX

SEQ ID NO: 14 - protein, 5 aa, Artificial Sequence
  CMV protease substrate consensus
VVXAS

SEQ ID NO: 15 - protein, 7 aa, Artificial Sequence
  CMV protease substrate consensus
VVNASCR

SEQ ID NO: 16 - protein, 7 aa, Artificial Sequence
  Herpes simplex virus protease substrate consensus
XXXASXX

SEQ ID NO: 17 - protein, 4 aa, Artificial Sequence
  Flavivirus protease substrate consensus
XXXX

SEQ ID NO: 18 - protein, 6 aa, Artificial Sequence
  Denguevirus protease substrate consensus
GXXXXX

SEQ ID NO: 19 - protein, 8 aa, Artificial Sequence
  Denguevirus protease substrate consensus
XXGRKSXT

SEQ ID NO: 20 - protein, 8 aa, Artificial Sequence
  Denguevirus protease substrate consensus
AAGRKSLT

SEQ ID NO: 21 - protein, 7 aa, Artificial Sequence
  West Nile virus protease substrate consensus
XXXXXXX

SEQ ID NO: 22 - protein, 9 aa, Artificial Sequence
  West Nile virus protease substrate consensus
PNRKRGWPA

SEQ ID NO: 23 - protein, 11 aa, Artificial Sequence
  West Nile virus protease substrate consensus
FASGKRSQIGV

SEQ ID NO: 24 - protein, 6 aa, Artificial Sequence
  Yellow fever virus protease substrate consensus
GXXXXX

SEQ ID NO: 25 - protein, 7 aa, Artificial Sequence
  Yellow fever virus protease substrate consensus
FGRRSIP

SEQ ID NO: 26 - protein, 7 aa, Artificial Sequence
  Yellow fever virus protease substrate consensus
EGRRGAA

SEQ ID NO: 27 - protein, 5 aa, Artificial Sequence
  Japanese Encephalitis virus protease substrate consensus
XXXXX

SEQ ID NO: 28 - protein, 8 aa, Artificial Sequence
  Japanese Encephalitis virus protease substrate consensus
NKKRGWPA

SEQ ID NO: 29 - protein, 9 aa, Artificial Sequence
  Japanese Encephalitis virus protease substrate consensus
AATGKRSAX

SEQ ID NO: 30 - protein, 6 aa, Artificial Sequence
  Tick bone virus protease substrate consensus
XXSXXX

SEQ ID NO: 31 - protein, 9 aa, Artificial Sequence
  Tick bone virus protease substrate consensus
RGRRSFSEV

SEQ ID NO: 32 - protein, 8 aa, Artificial Sequence
  Tick bone virus protease substrate consensus
SGRRSFGD

SEQ ID NO: 33 - protein, 9 aa, Artificial Sequence
  Hepatitis C NS3 Protease substrate consensus
XXXXCXXXX

SEQ ID NO: 34 - protein, 8 aa, Artificial Sequence
  Hepatitis C NS2-3 Protease substrate consensus
XXXXXXXX

SEQ ID NO: 35 - protein, 8 aa, Artificial Sequence
  Hepatitis C NS2-3 Protease substrate consensus
GXXLLXPI

SEQ ID NO: 36 - protein, 7 aa, Artificial Sequence
  Pestivirus protease substrate consensus
XXXLXXX

SEQ ID NO: 37 - protein, 9 aa, Artificial Sequence
  Hepatitis A, 3C protease substrate consensus
XWSQGISXD

SEQ ID NO: 38 - protein, 7 aa, Artificial Sequence
  Hepatitis A, 3C protease substrate consensus
EFFQSFP

SEQ ID NO: 39 - protein, 4 aa, Artificial Sequence
  HRV 3C protease substrate consensus
FQGP

SEQ ID NO: 40 - protein, 4 aa, Artificial Sequence
  HRV 2A protease substrate consensus
XXGX

SEQ ID NO: 41 - protein, 4 aa, Artificial Sequence
  Enterovirus 3C protease substrate consensus
FQGP

SEQ ID NO: 42 - protein, 8 aa, Artificial Sequence
  Coxsackievirus protease substrate consensus
TTGXXXQQ

SEQ ID NO: 43 - protein, 6 aa, Artificial Sequence
  Coxsackievirus protease substrate consensus
AXGXQX

SEQ ID NO: 44 - protein, 8 aa, Artificial Sequence
  Echovirus protease substrate consensus
XTGXXXQQ

SEQ ID NO: 45 - protein, 8 aa, Artificial Sequence
  Echovirus protease substrate consensus
XXGAFXQQ

SEQ ID NO: 46 - protein, 6 aa, Artificial Sequence
  Echovirus protease substrate consensus
XFGQQX

SEQ ID NO: 47 - protein, 9 aa, Artificial Sequence
  Echovirus protease substrate consensus
XXGXFXQQS

SEQ ID NO: 48 - DNA, 20 bp, Artificial Sequence
  Single strand DNA oligonucleotide
caccggtccagaagaagaat

SEQ ID NO: 49 - DNA, 22 bp, Artificial Sequence
  Single strand DNA oligonucleotide
tcattgttgttcagtgaagtat

SEQ ID NO: 50 - DNA, 24 bp, Artificial Sequence
  Single strand DNA oligonucleotide
caccagtggttttaggaaaatggc

SEQ ID NO: 51 - DNA, 23 bp, Artificial Sequence
  Single strand DNA oligonucleotide
ttattggaaggtaacaccagagc

SEQ ID NO: 52 - protein, 11 aa, Artificial Sequence
  Hepatitis C NS3 protease cleavage sequence
PVSARRGKEIF

SEQ ID NO: 53 - protein, 11 aa, Artificial Sequence
  Hepatitis C NS3 protease cleavage sequence
PVSARREREIL

SEQ ID NO: 54 - protein, 11 aa, Artificial Sequence
  Hepatitis C NS3 protease cleavage sequence
QGWRLLAPITA

SEQ ID NO: 55 - protein, 11 aa, Artificial Sequence
  Hepatitis C NS3 protease cleavage sequence
PVSARRGREIL

SEQ ID NO: 56 - protein, 11 aa, Artificial Sequence
  Hepatitis C NS3 protease cleavage sequence
GGWKLLAPITA

SEQ ID NO: 57 - protein, 11 aa, Artificial Sequence
  Hepatitis C NS3 protease cleavage sequence
PVSARRGREVL

SEQ ID NO: 58 - protein, 11 aa, Artificial Sequence
  Hepatitis C NS3 protease cleavage sequence
KGWKLLAPITA

SEQ ID NO: 59 - protein, 11 aa, Artificial Sequence
  Hepatitis C NS3 protease cleavage sequence
PVSARLGREVL

SEQ ID NO: 60 - protein, 11 aa, Artificial Sequence
  Hepatitis C NS3 protease cleavage sequence
PVSARLGRELL

SEQ ID NO: 61 - protein, 11 aa, Artificial Sequence
  Hepatitis C NS3 protease cleavage sequence
PVSARLGQEVL

SEQ ID NO: 62 - protein, 11 aa, Artificial Sequence
  Hepatitis C NS3 protease cleavage sequence
QGWRLLAHITA

SEQ ID NO: 63 - protein, 11 aa, Artificial Sequence
  Hepatitis C NS3 protease cleavage sequence
CLIRLKPTLHG

SEQ ID NO: 64 - protein, 11 aa, Artificial Sequence
  Hepatitis C NS3 protease cleavage sequence
DLEVVTSTWVL

SEQ ID NO: 65 - protein, 11 aa, Artificial Sequence
  Hepatitis C NS3 protease cleavage sequence
LTIPASAYEVR

SEQ ID NO: 66 - protein, 11 aa, Artificial Sequence
  Hepatitis C NS3 protease cleavage sequence
TIPASAYEVCN

SEQ ID NO: 67 - protein, 11 aa, Artificial Sequence
  Hepatitis C NS3 protease cleavage sequence
TVPASAYQVRN

SEQ ID NO: 68 - protein, 11 aa, Artificial Sequence
  Hepatitis C NS3 protease cleavage sequence
TIPASAYQVRN

SEQ ID NO: 69 - protein, 11 aa, Artificial Sequence
  Hepatitis C NS3 protease cleavage sequence
TIPASAYEVRN

SEQ ID NO: 70 - protein, 11 aa, Artificial Sequence
  Hepatitis C NS3 protease cleavage sequence
TVPVSTYEVRN

SEQ ID NO: 71 - protein, 11 aa, Artificial Sequence
  Hepatitis C NS3 protease cleavage sequence
TVPASAYEVRN

SEQ ID NO: 72 - protein, 11 aa, Artificial Sequence
  Hepatitis C NS3 protease cleavage sequence
TVPASAYHVRN

SEQ ID NO: 73 - protein, 11 aa, Artificial Sequence
  Hepatitis C NS3 protease cleavage sequence
TTPASALTYGN

SEQ ID NO: 74 - protein, 11 aa, Artificial Sequence
  Hepatitis C NS3 protease cleavage sequence
TVPASAVGVRN

SEQ ID NO: 75 - protein, 11 aa, Artificial Sequence
  Hepatitis C NS3 protease cleavage sequence
TVPVSSVEIRN

SEQ ID NO: 76 - protein, 11 aa, Artificial Sequence
  Hepatitis C NS3 protease cleavage sequence
IHPAASLEWRN

SEQ ID NO: 77 - protein, 11 aa, Artificial Sequence
  Hepatitis C NS3 protease cleavage sequence
TCPASSLEYRN

SEQ ID NO: 78 - protein, 11 aa, Artificial Sequence
  Hepatitis C NS3 protease cleavage sequence
SVPVSAVEVKN

SEQ ID NO: 79 - protein, 11 aa, Artificial Sequence
  Hepatitis C NS3 protease cleavage sequence
FAGVDGNTYTT

SEQ ID NO: 80 - protein, 11 aa, Artificial Sequence
  Hepatitis C NS3 protease cleavage sequence
FAGVDGNTYVS

SEQ ID NO: 81 - protein, 11 aa, Artificial Sequence
  Hepatitis C NS3 protease cleavage sequence
FVGVDGSTHVS

SEQ ID NO: 82 - protein, 11 aa, Artificial Sequence
  Hepatitis C NS3 protease cleavage sequence
FAGVDGDTYTT

SEQ ID NO: 83 - protein, 11 aa, Artificial Sequence
  Hepatitis C NS3 protease cleavage sequence
FAGVDGRTTVT

SEQ ID NO: 84 - protein, 11 aa, Artificial Sequence
  Hepatitis C NS3 protease cleavage sequence
FVGVDGSTRVS

SEQ ID NO: 85 - protein, 11 aa, Artificial Sequence
  Hepatitis C NS3 protease cleavage sequence
FAGVDAETHVT

SEQ ID NO: 86 - protein, 11 aa, Artificial Sequence
  Hepatitis C NS3 protease cleavage sequence
FAGVDGETYTS

SEQ ID NO: 87 - protein, 11 aa, Artificial Sequence
  Hepatitis C NS3 protease cleavage sequence
FAGVDGATYTS

SEQ ID NO: 88 - protein, 11 aa, Artificial Sequence
  Hepatitis C NS3 protease cleavage sequence
FAGVDGDTHTT

SEQ ID NO: 89 - protein, 11 aa, Artificial Sequence
  Hepatitis C NS3 protease cleavage sequence
FAGVDGTTYVS

SEQ ID NO: 90 - protein, 11 aa, Artificial Sequence
  Hepatitis C NS3 protease cleavage sequence
FAGVDGTTTVS

SEQ ID NO: 91 - protein, 11 aa, Artificial Sequence
  Hepatitis C NS3 protease cleavage sequence
FAGVDGQTRVT

SEQ ID NO: 92 - protein, 11 aa, Artificial Sequence
  Hepatitis C NS3 protease cleavage sequence
FAGVDANTYVT

SEQ ID NO: 93 - protein, 11 aa, Artificial Sequence
  Hepatitis C NS3 protease cleavage sequence
FAGVEATTTVG

SEQ ID NO: 94 - protein, 11 aa, Artificial Sequence
  Hepatitis C NS3 protease cleavage sequence
FAGVDAETRVT

SEQ ID NO: 95 - protein, 11 aa, Artificial Sequence
  Hepatitis C NS3 protease cleavage sequence
VAGVDATTYST

SEQ ID NO: 96 - protein, 11 aa, Artificial Sequence
  Hepatitis C NS3 protease cleavage sequence
FSGVDAHTYTT

SEQ ID NO: 97 - protein, 11 aa, Artificial Sequence
  Hepatitis C NS3 protease cleavage sequence
FSGVDATTHTT

SEQ ID NO: 98 - protein, 11 aa, Artificial Sequence
  Hepatitis C NS3 protease cleavage sequence
TAGVDAQTHTI

SEQ ID NO: 99 - protein, 11 aa, Artificial Sequence
  Hepatitis C NS3 protease cleavage sequence
FSGVDAETYIT

SEQ ID NO: 100 - protein, 11 aa, Artificial Sequence
  Hepatitis C NS3 protease cleavage sequence
ISQAEAALENL

SEQ ID NO: 101 - protein, 11 aa, Artificial Sequence
  Hepatitis C NS3 protease cleavage sequence
IAQAEAALENL

SEQ ID NO: 102 - protein, 11 aa, Artificial Sequence
  Hepatitis C NS3 protease cleavage sequence
ISNVEAAVERL

SEQ ID NO: 103 - protein, 11 aa, Artificial Sequence
  Hepatitis C NS3 protease cleavage sequence
LGQAEAALEKL

SEQ ID NO: 104 - protein, 11 aa, Artificial Sequence
  Hepatitis C NS3 protease cleavage sequence
IAQAEATLENL

SEQ ID NO: 105 - protein, 11 aa, Artificial Sequence
  Hepatitis C NS3 protease cleavage sequence
PQRAYALDTEM

SEQ ID NO: 106 - protein, 11 aa, Artificial Sequence
  Hepatitis C NS3 protease cleavage sequence
DARVCACLWMM

SEQ ID NO: 107 - protein, 11 aa, Artificial Sequence
  Hepatitis C NS3 protease cleavage sequence
PQRAYALDTEV

SEQ ID NO: 108 - protein, 11 aa, Artificial Sequence
  Hepatitis C NS3 protease cleavage sequence
PPRAYALDREM

SEQ ID NO: 109 - protein, 11 aa, Artificial Sequence
  Hepatitis C NS3 protease cleavage sequence
PHRAYAMDNEQ

SEQ ID NO: 110 - protein, 11 aa, Artificial Sequence
  Hepatitis C NS3 protease cleavage sequence
PQRAYALDQEL

SEQ ID NO: 111 - protein, 11 aa, Artificial Sequence
  Hepatitis C NS3 protease cleavage sequence
PQQAYALDAAE

SEQ ID NO: 112 - protein, 11 aa, Artificial Sequence
  Hepatitis C NS3 protease cleavage sequence
LLLADARVCVA

SEQ ID NO: 113 - protein, 11 aa, Artificial Sequence
  Hepatitis C NS3 protease cleavage sequence
PPRAYAMDREM

SEQ ID NO: 114 - protein, 11 aa, Artificial Sequence
  Hepatitis C NS3 protease cleavage sequence
PPQAYAMDREM

SEQ ID NO: 115 - protein, 11 aa, Artificial Sequence
  Hepatitis C NS3 protease cleavage sequence
DLEIVTSTWVL

SEQ ID NO: 116 - protein, 11 aa, Artificial Sequence
  Hepatitis C NS3 protease cleavage sequence
DLEVITSTWVL

SEQ ID NO: 117 - protein, 11 aa, Artificial Sequence
  Hepatitis C NS3 protease cleavage sequence
DLEVMTSTWVL

SEQ ID NO: 118 - protein, 11 aa, Artificial Sequence
  Hepatitis C NS3 protease cleavage sequence
DLEVTTSTWVL

SEQ ID NO: 119 - protein, 11 aa, Artificial Sequence
  Hepatitis C NS3 protease cleavage sequence
DLEVTTSAWVL

SEQ ID NO: 120 - protein, 11 aa, Artificial Sequence
  Hepatitis C NS3 protease cleavage sequence
DLEIMTSTWVL

SEQ ID NO: 121 - protein, 11 aa, Artificial Sequence
  Hepatitis C NS3 protease cleavage sequence
DEMEECSQHLP

SEQ ID NO: 122 - protein, 11 aa, Artificial Sequence
  Hepatitis C NS3 protease cleavage sequence
AGVAGALVAFK

SEQ ID NO: 123 - protein, 11 aa, Artificial Sequence
  Hepatitis C NS3 protease cleavage sequence
DEMEECASHLP

SEQ ID NO: 124 - protein, 11 aa, Artificial Sequence
  Hepatitis C NS3 protease cleavage sequence
DEMEECSRHIP

SEQ ID NO: 125 - protein, 11 aa, Artificial Sequence
  Hepatitis C NS3 protease cleavage sequence
DEMEECASKAA

SEQ ID NO: 126 - protein, 11 aa, Artificial Sequence
  Hepatitis C NS3 protease cleavage sequence
DEMEECSQAAP

SEQ ID NO: 127 - protein, 11 aa, Artificial Sequence
  Hepatitis C NS3 protease cleavage sequence
DEMEECASRAL

SEQ ID NO: 128 - protein, 11 aa, Artificial Sequence
  Hepatitis C NS3 protease cleavage sequence
ECTTPCSGSWL

SEQ ID NO: 129 - protein, 11 aa, Artificial Sequence
  Hepatitis C NS3 protease cleavage sequence
DCSTPCSGSWL

SEQ ID NO: 130 - protein, 11 aa, Artificial Sequence
  Hepatitis C NS3 protease cleavage sequence
ESTTPCSGSWL

SEQ ID NO: 131 - protein, 11 aa, Artificial Sequence
  Hepatitis C NS3 protease cleavage sequence
DTATPCATSWL

SEQ ID NO: 132 - protein, 11 aa, Artificial Sequence
  Hepatitis C NS3 protease cleavage sequence
DCTAPCAGSWL

SEQ ID NO: 133 - protein, 11 aa, Artificial Sequence
  Hepatitis C NS3 protease cleavage sequence
DCPVPCSGSWL

SEQ ID NO: 134 - protein, 11 aa, Artificial Sequence
  Hepatitis C NS3 protease cleavage sequence
DYPSPCSDDWL

SEQ ID NO: 135 - protein, 11 aa, Artificial Sequence
  Hepatitis C NS3 protease cleavage sequence
DYPSPCNGDWL

SEQ ID NO: 136 - protein, 11 aa, Artificial Sequence
  Hepatitis C NS3 protease cleavage sequence
EDVVCCSMSYT

SEQ ID NO: 137 - protein, 11 aa, Artificial Sequence
  Hepatitis C NS3 protease cleavage sequence
DDVVCCSMSYS

SEQ ID NO: 138 - protein, 11 aa, Artificial Sequence
  Hepatitis C NS3 protease cleavage sequence
QSVVCCSMSYS

SEQ ID NO: 139 - protein, 11 aa, Artificial Sequence
  Hepatitis C NS3 protease cleavage sequence
GSEVCCSMSYS

SEQ ID NO: 140 - protein, 11 aa, Artificial Sequence
  Hepatitis C NS3 protease cleavage sequence
DDIVCCSMSYT

SEQ ID NO: 141 - protein, 11 aa, Artificial Sequence
  Hepatitis C NS3 protease cleavage sequence
EDVVCCSMSYS

SEQ ID NO: 142 - protein, 11 aa, Artificial Sequence
  Hepatitis C NS3 protease cleavage sequence
DSVICCSMSYS

SEQ ID NO: 143 - protein, 12 aa, Artificial Sequence
  Synthetic HRV 3C protease substarate
SAIFQGPISMDK

SEQ ID NO: 144 - protein, 11 aa, Artificial Sequence
  Synthetic HRV 3C protease substarate
LEALFQGPDSQ

SEQ ID NO: 145 - protein, 5 aa, Artificial Sequence
  Synthetic HRV 3C protease substarate
EALFQ

SEQ ID NO: 146 - protein, 8 aa, Artificial Sequence
  Synthetic HRV 3C protease substarate
DSLEVLFQ

SEQ ID NO: 147 - protein, 11 aa, Artificial Sequence
  Synthetic HRV 3C protease substarate
LEVLFQGPDSQ

SEQ ID NO: 148 - protein, 12 aa, Artificial Sequence
  Synthetic HRV 3C protease substarate
TSAVLQSGFRDK

SEQ ID NO: 149 - protein, 6 aa, Artificial Sequence
  Synthetic HRV 3C protease substarate
TSAVLQ

SEQ ID NO: 150 - protein, 11 aa, Artificial Sequence
  Synthetic HRV 3C protease substarate
LEALFQAADSQ

SEQ ID NO: 151 - protein, 11 aa, Artificial Sequence
  Synthetic HRV 3C protease substarate
LEALFQGPDSQ

(19) United States
(12) Patent Application Publication
(10) Pub. No.: US 2009/0281042 A1
(43) Pub. Date: Nov. 12, 2009

(54) COMPOSITIONS AND METHODS USING SAME FOR THE DETECTION OF VIRUSES

(76) Inventors: Assaf Ezra, Bat-yam (IL); Dorit Arad, Tel-aviv (IL); Gilad Wainreb, Tel-aviv (IL)

Correspondence Address: CANTOR COLBURN, LLP, 20 Church Street, 22nd Floor, Hartford, CT, 06103, US

(21) Appl. No.: 12/066,312

(22) PCT Filed: Sep. 10, 2006
(86) PCT No.: PCT/IL06/01055

§ 371 Date: Oct. 16, 2008

Related U.S. Application Data
(60) Provisional application No. 60/714,760, filed on Sep. 8, 2005.
(60) Provisional application No. 60/729,752, filed on Oct. 25, 2005.

(51) Int. Cl. A61K 38/08; C07K 5/08; C07K 7/06; C12N 9/50; A61K 38/06; A61P 31/12

(52) U.S. Cl. 514/15; 530/330; 530/329; 530/331; 530/328; 435/219; 435/5; 514/18; 514/17; 514/16

(57) ABSTRACT

An isolated peptide is provided. The isolated peptide comprising an amino acid sequence selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 and 47, said amino acid sequence being no more than 14 amino acids in length. Also provided are compositions which comprise the peptides and use of same in the detection of viruses.

### FIELD AND BACKGROUND OF THE INVENTION

The present invention relates to novel compositions for the simple and rapid detection of a cleavage activity of a catalytic molecule. Diagnostic tests and kits using these compositions are also provided.

There is an increasing need for rapid, specific and cost-effective detection of health-related agents, whether markers of disease or health risk, markers or agents of normal and pathogenic processes or disease, or indicators of foreign pathogens and their byproducts. This is made ever more evident by the repeated incidence of spread of contagious disease throughout the world, such as HIV, SARS, Avian Flu, West Nile Fever, resistant pathogenic bacterial diseases, etc. In the face of a growing threat of epidemic and pandemic outbreaks of disease, early detection of disease agents has become crucial to adequate care and prevention. However, the cost, complexity and inefficiency of many currently available methods of pathogen detection and typing have forced the implementation of time consuming, labor intensive, ultimately costly and inefficient policies, such as quarantine, vector eradication and prophylactic vaccination, with questionable success. Further, numerous markers of disease and pathological processes have been identified, some of which comprise catalytic activity, such as teleomerase in various cancers, Terminal Deoxynucleotidyl Transferase (TdT) in leukemias and β- and γ-secretase in Alzheimer's disease.

In 1918 the world experienced the Spanish flu, killing more than 25 million people in Europe. Since then the fear of medical emergency has returned. Just a few years ago, the deadly SARS virus erupted in the East, threatening to paralyze the world's economies. The SARS outbreak was slowed and eventually overcome, but only after extremely disruptive measures, including lengthy quarantine of healthy and sick people alike that were isolated for no more than a fever. Currently, the outbreak of Avian Flu threatens to once again remind the world of the importance of improved methods for the rapid detection of dangerous pathogens.

In the 21 century the danger of spread of a new pandemic is enormously high due to the high likelihood of infection in air travel, and the increased mobility of individuals, and populations. Methods currently in use for detection of pathogenic agents include immunological detection, such as enzyme linked immunosorbent assay (ELISA), enzyme immunoassay (EIA) and immunofluorescence assay (IFA), which rely on detection of anti-pathogen antibodies, 10-20 days after initial infection, RT-PCR, which is costly, time-consuming and often inaccurate, and tissue culture infectivity, also time-consuming and costly. Therefore there is an urgent need for rapid and simple diagnostic methods and kits for detection of afflicted, and potentially infectious individuals in airports and other public places. The method must be capable of accurate detection, in the face of the highly variable character of current viral agents, able to mutate and form highly pathogenic species. There is a need to detect several viral pathogens at once, in order to exclude the possibility of mixing cross infectivity and production of more violent types of viruses. Rapid and accurate typing of the pathogenic agents is also a priority. There is a need for rapid detection of a number of pathogenic agents, in the hospital setting or at home, which would provide accurate data for choice of treatment, prevent mixing and infectivity of different pathogenic agents as well, and facilitate on-site monitoring and fine tuning of treatment protocols.

Many organisms have characteristic catalytic activity associated with specific stages of growth or differentiation, metabolism, etc. Likewise, pathological processes often have characteristic enzymatic activities which can be employed in their diagnosis, such as cancer markers and cardiac enzymes. Methods based on detection of catalytic activity for diagnostics and classification have been disclosed—for example, for diagnosis and classification of pathogenic bacteria (see, for example, Maiden et al, J. Clin. Micro, 1996; 34:376-84 and U.S. Pat. No. 5,888,760 to Godsey, et al), DNA-phytolyases in carcinogenesis (see, for example, Kundu et al ChemBioChem 2002; 3:1053-60), β-secretase activity in Alzheimer's disease (see US Patent Application No. 200302555 to Hazuda et al), mapping of metabolic activity in living cells (see Boonacker et al, J of Histochem and Cytochem 2001; 49:1473-86), caspase and other apoptosis-related enzymes (see US Patent Application No. 20020150885, to Weber et al) and detection of viruses (see, for example, U.S. Pat. No. 4,952,493 to Kettner, et al).

Efficient measurement and clinical use of such catalytic activity requires well defined, specific substrates which can be made readily detectable. One such potentially diagnostic catalytic activity is the specific protease activity of viral infection.

During the replication of many viruses, such as the SARS virus, human immunodeficiency virus, human papilloma virus, herpes virus, rhinovirus, picornavirus, coronavirus, hepatitis C virus, and others, the viral genetic material is transcribed to form a polyprotein, which is ultimately cleaved into two or more biologically active proteins. The cleavage of the viral polyprotein into individual proteins is a critical part of the viral life cycle. Many viruses, including those of the adenovirus, baculovirus, comovirus, picornavirus, retrovirus, and togavirus families, encode proteases which cleave the viral polyprotein at these specific cleavage positions to form the active proteins required for viral replication. For example, polyprotein processing during replication of the Cocksfoot mottle virus (genus Sobemovirus) is described in Makinen, K. et al., J. Gen. Virol. 2000; 81:2783-89, the contents of which are incorporated herein by reference. For reference, a non-exhaustive listing of some known examples of viral proteases and other enzymes is provided hereinbelow.

Some virally-encoded proteases cleave only the polyprotein of a specific virus. Others cleave the polyprotein of more than one type of virus. The specificity of protease action arises from the nature of the interaction of the protease at the cleavage region(s) of the polyprotein. In addition, the rate of cleavage at these positions varies, depending on the peptide sequence of the polyprotein surrounding the cleavage position.

The cleavage sites along the viral proteins that are recognized by these viral proteases have been shown to contain highly conserved amino acid sequences, which suggests the possibility of their incorporation into diagnostic and therapeutic methods.

For example, U.S. Pat. No. 4,952,493 to Kettner et al. discloses peptide substrates for detection of viral-specific protease activity, designed according to conserved cleavage sites recognized by viral proteases. The cleavage sites of these peptide substrates are established according to amino acid sequences of viral-specific cleavage sites, determined by sequencing of viral polypeptides, or according to the coding sequences of the viral genome, and can be compared by alignment with other viral polypeptide sequences. Conservative substitutions of certain amino acid residues with other, biologically similar residues is considered tolerable.

US Patent Application No. 20050214890 to Tan et al. discloses the use of matrix-bound recombinant fluorescent fusion substrates for the detection of parasitic, protozoan, viral and other protease activity in a sample, wherein the detection is based on the pattern of protease recognition of multiple substrates. The cleavage and/or recognition substrates are designed from known consensus cleavage and/or binding sequences. Enhanced detection of target proteases is due to the simultaneous assay of multiple substrates.

However, none of the above described methods describe, suggest or mention selecting the viral substrates such that optimized affinity is obtained ultimately resulting in rapid simultaneous analysis of multiple samples, and multiple viruses as well as specific recognition of novel strains.

Improved efficiency of the protease detection assay by optimized design of the substrate cleavage sequences would provide for superior rapidity, sensitivity and economy of detection and characterization of viral infections. Further, methods for the design of substrate cleavage sequences, and the products thereof can be used in the screening and development of anti-viral pharmaceuticals.

There is thus a widely recognized need for, and it would be highly advantageous to have optimized substrates and methods for detecting cleavage activity of catalytic molecules for rapid, specific detection of disease and infective processes characterized by cleavage activity, devoid of the above limitations.

### SUMMARY OF THE INVENTION

According to one aspect of the present invention there is provided an isolated peptide comprising an amino acid sequence selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 and 47, the amino acid sequence being no more than 14 amino acids in length.

According to another aspect of the present invention there is provided a composition comprising a substrate of a viral protease attached to at least one detactable moiety, the substrate comprising the amino acid sequence.

According to further features in preferred embodiments of the invention described below, the at least one detectable moiety is a FRET pair, and whereas cleavage of the substrate generates a signal from the FRET pair.

According to further features in preferred embodiments of the invention described below, the composition further comprising a separating moiety.

According to yet another aspect of the present invention there is provided a composition being of the general formula:

X-Y-Z

wherein:

Y comprises a substrate of a viral protease the substrate comprising the amino acid sequence, cleavage of X-Y-Z by the viral protease forming cleavage products X-Y′ and Y″-Z wherein Y′ is a first cleavage product of Y and Y″ is a second cleavage product of Y;

X comprises a detectable moiety; and

Z comprises a separating moiety capable of binding to a separate phase of a two phase separating system;

wherein the X-Y-Z does not form a contiguous portion of a natural substrate the viral protease.

According to further features in preferred embodiments of the invention described below, the detectable moiety X comprises a labeling agent selected from the group consisting of an enzyme, a fluorophore, a chromophore, a protein, a pro-enzyme, a chemiluminescent substance and a radioisotope.

According to further features in preferred embodiments of the invention described below, the separating moiety Z is selected from the group consisting of an immunological binding agent, a magnetic binding moiety, a peptide binding moiety, an affinity binding moiety, a nucleic acid moiety.

According to still another aspect of the present invention there is provided a composition being of the general formula:

X-Y-Z

wherein:

Y comprises a substrate of a viral protease the substrate comprising the amino acid sequence, cleavage of X-Y-Z by the viral protease forming cleavage products X-Y′ and Y″-Z wherein Y′ is a first cleavage product of Y and Y″ is a second cleavage product of Y;

X or Z comprises a marker, either a detectable moiety and/or a separating moiety capable of separating between cleaved and uncleaved composition in a suited manner.

wherein the X-Y-Z does not form a contiguous portion of a natural substrate the viral protease.

According to further features in preferred embodiments of the invention described below, wherein the marker, moiety X or Z, comprises a labeling agent selected from the group consisting of an enzyme, a fluorophore, a chromophore, a protein, a chemiluminescent substance, a quencher, a FRET pair, a bead, a peptide, a pre-enzyme and a radioisotope, an immunological binding agent, a magnetic binding moiety, a peptide binding moiety, an affinity binding moiety, a nucleic acid moiety.

According to an additional aspect of the present invention there is provided a method for detecting at least one virus in a sample, the method comprising
(a) contacting the sample with at least one of the compositions under conditions allowing cleavage of the substrate; and
(b) monitoring cleavage of the substrate, wherein the cleavage of the substrate is indicative of the presence of the at least one virus in the sample.

According to further features in preferred embodiments of the invention described below, step (a) comprises contacting the sample with at least two substrates of different viral proteases, wherein absence of the cleavage of any of the at least two substrates indicative of the absence of a virus from the sample.

According to further features in preferred embodiments of the invention described below, the sample is selected from the group consisting of mucus, saliva, throat wash, nasal wash, spinal fluid, sputum, urine, semen, sweat, feces, plasma, blood, bronchioalveolar fluid, vaginal fluid, tear fluid and tissue biopsy.

According to further features in preferred embodiments of the invention described below, detection of the cleavage activity in the sample is diagnostic of a medical condition.

According to further features in preferred embodiments of the invention described below, the monitoring is effected using a homogeneous assay.

According to further features in preferred embodiments of the invention described below, the monitoring is effected using a heterogeneous assay.

According to yet an additional aspect of the present invention there is provided a diagnostic kit for detection of at least one virus in a sample, the kit comprising at least one composition, and reagents for detecting cleavage of the substrate.

According to still an additional aspect of the present invention there is provided a diagnostic kit comprising a packaging material and a plurality compositions for detecting presence of a plurality of viruses, wherein each of the compositions is of a general formula,

X-Y-Z

wherein:

Y comprises a substrate of a viral protease, cleavage of X-Y-Z by the viral protease forming cleavage products X-Y′ and Y″-Z wherein Y′ is a first cleavage product of Y and Y″ is a second cleavage product of Y;

X or Z comprises a marker, either a detectable moiety and/or a separating moiety capable of separating between cleaved and uncleaved compositions in a suited manner;

wherein the X-Y-Z does not form a contiguous portion of a natural substrate the viral protease,

wherein each of the X or Z comprise of at least one distinctively detectable moiety and whereas the packaging material comprises a label or package insert indicating that the kit is for detection of plurality of viruses in a sample.

According to a further aspect of the present invention there is provided a diagnostic kit comprising a packaging material and a plurality of compositions for detecting presence of a plurality of viruses, wherein each of the compositions is of a general formula,

X-Y-Z

wherein:

Y comprises a substrate of a viral protease, cleavage of X-Y-Z by the viral protease forming cleavage products X-Y′ and Y″-Z wherein Y′ is a first cleavage product of Y and Y″ is a second cleavage product of Y;

X comprises a detectable moiety; and

Z comprises a separating moiety capable of binding to a separate phase of a two phase separating system;

wherein the X-Y-Z does not form a contiguous portion of a natural substrate the viral protease,

wherein each of the X is distinctively detectable and whereas the packaging material comprises a label or package insert indicating that the kit is for detection of plurality of viruses in a sample.

According to further features in preferred embodiments of the invention described below, the plurality of compositions are attached to a single solid support.

According to further features in preferred embodiments of the invention described below, the distinctive detection is effected by an addressable location on the single solid support.

According to further features in preferred embodiments of the invention described below, the distinctive detection is effected by different detectable moieties.

According to further features in preferred embodiments of the invention described below, each of the plurality of compositions is attached to a solid support.

According to further features in preferred embodiments of the invention described below, wherein the solid support is configured as a bead.

According to further features in preferred embodiments of the invention described below, the bead is selected from the group consisting of a colored bead, a magnetic bead, a tagged bead and a fluorescent bead.

According to further features in preferred embodiments of the invention described below, is a respiratory kit comprising at least two viruses selected from group consisting of Corona Viruses, SARS, HMPV (Human Meta pneumo virus), Influenza A+B, Avian Influenza, Adeno virus, RSV (Respiratory Syncytial Virus), Rhino virus, Para influenza viruses.

According to further features in preferred embodiments of the invention described below, is a respiratory kit comprising Hanta virus and La Crosse Encephalitis.

According to further features in preferred embodiments of the invention described below, is a gastrointestinal kit comprising at least two viruses selected from group consisting of Rota virus, Adeno 40/41, Hepatitis A, Hepatitis C, Hepatitis E, caliciviruses and CMV (Cytomegalovirus).

According to further features in preferred embodiments of the invention described below, the diagnostic kit is a meningitis kit comprising at least two viruses selected from group consisting of Enteroviruses (1-˜80), West Nile virus, Herpes Simplex 1, 2, and 6.

According to further features in preferred embodiments of the invention described below, the diagnostic kit, is a meningitis kit comprising at least two viruses selected from group consisting of a Toga virus, Flavi virus and Rabies.

According to further features in preferred embodiments of the invention described below, the diagnostic kit is a sexually transmitted diseases kit comprising at least two viruses selected from group consisting of HIV strain, Herpes simplex 1, Herpes simplex 2, HSV-1, HSV-2, HPV (Human Papilloma Viruses), and HTLV-1.

According to further features in preferred embodiments of the invention described below, the diagnostic kit is a Traveler's kit comprising at least two viruses selected from group consisting of Hepatitis A, Hepatitis B, Hepatitis C, HIV, Herpes Virus 1 and 2.

According to further features in preferred embodiments of the invention described below, the diagnostic kit is a veterinarian kit comprising at least two viruses selected from group consisting of Rabies and Distemper.

According to further features in preferred embodiments of the invention described below, the at least one sample comprises a plurality of samples.

According to further features in preferred embodiments of the invention described below, the at least one virus comprises a plurality of viruses.

According to further features in preferred embodiments of the invention described below, the virus is adenovirus and the substrate comprises SEQ ID NO: 1 or 2.

According to further features in preferred embodiments of the invention described below, the virus is alphavirus and the substrate comprises SEQ ID NO: 3.

According to further features in preferred embodiments of the invention described below, the virus is Rubella virus and the substrate comprises SEQ ID NO: 4.

According to further features in preferred embodiments of the invention described below, the virus is HIV and the substrate comprises SEQ ID NO: 5.

According to further features in preferred embodiments of the invention described below, the virus is HTLV and the substrate comprises SEQ ID NO: 6, 7 or 8.

According to further features in preferred embodiments of the invention described below, the virus is Arteri virus and the substrate comprises SEQ ID NO: 9.

According to further features in preferred embodiments of the invention described below, the virus is Corona virus and the substrate comprises SEQ ID NO: 10.

According to further features in preferred embodiments of the invention described below, the virus is SARS corona virus is and the substrate comprises SEQ ID NO: 11 or 12.

According to further features in preferred embodiments of the invention described below, the virus is Torovirus virus and the substrate comprises SEQ ID NO: 13.

According to further features in preferred embodiments of the invention described below, the virus is CMV virus and the substrate comprises SEQ ID NO: 14, or 15.

According to further features in preferred embodiments of the invention described below, the virus is Herpes virus and the substrate comprises SEQ ID NO: 16.

According to further features in preferred embodiments of the invention described below, the virus is Flavivirus virus and the substrate comprises SEQ ID NO: 17.

According to further features in preferred embodiments of the invention described below, the virus is Denguevirus virus and the substrate comprises SEQ ID NO: 18, 19 or 20.

According to further features in preferred embodiments of the invention described below, the virus is West Nile virus and the substrate comprises SEQ ID NO: 21, 22 or 23.

According to further features in preferred embodiments of the invention described below, the virus is Yellow fever virus and the substrate comprises SEQ ID NO: 24, 25 or 26.

According to further features in preferred embodiments of the invention described below, the virus is Japanese Encephalitis virus and the substrate comprises SEQ ID NO: 27, 28 or 29.

According to further features in preferred embodiments of the invention described below, wherein the virus is Tick bone virus and the substrate comprises SEQ ID NO: 30, 31 or 32.

According to further features in preferred embodiments of the invention described below, the virus is Hepatitis C virus and the substrate comprises SEQ ID NO: 33, 34 or 35.

According to further features in preferred embodiments of the invention described below, the virus is Pestivirus and the substrate comprises SEQ ID NO: 36.

According to further features in preferred embodiments of the invention described below, the virus is Hepatitis A virus and the substrate comprises SEQ ID NO: 37 or 38.

According to further features in preferred embodiments of the invention described below, the virus is HRV and the substrate comprises SEQ ID NO: 39 or 40.

According to further features in preferred embodiments of the invention described below, the virus is Enterovirus and the substrate comprises SEQ ID NO: 41, 42, 43, 44, 45, 46 or 47.

According to further features in preferred embodiments of the invention described below, the virus is an HRV virus and the substrate comprises SEQ ID NOs: 143-151.

According to yet a further aspect of the present invention there is provided a method for designing a kinetically optimal substrate for a protease of a virus, the method comprising:
(a) identifying in a plurality of cleavage sequences of a polyprotein of at least one strain of the virus, a cleavage sequence displaying most rapid cleavage kinetics by the protease, and
(b) identifying a family-wide consensus cleavage sequence displaying most rapid cleavage kinetics, the family-wide consensus cleavage sequence being useful for designing the kinetically optimal substrate for the protease of the virus.

According to further features in preferred embodiments of the invention described below, the protease of a virus is a viral encoded protease.

According to further features in preferred embodiments of the invention described below, the virus is selected from the group consisting of a DNA virus and an RNA virus.

According to further features in preferred embodiments of the invention described below, the virus is selected from the group consisting of Tectiviridae, Papovaviridae, Circoviridae, Parvoviridae, and Hepadnaviridae, Cystoviridae, Birnaviridae, Reoviridae, Coronaviridae, Flaviviridae, Togaviridae, Arterivirus, Astroviridae, Caliciviridae, Picornaviridae, Potyviridae, Retroviridae, Orthomyxoviridae, Filoviridae, Paramyxoviridae, Rhabdoviridae and Bunyaviridae, Adenoviridae, Herpesviridae, Picornaviridae.

According to further features in preferred embodiments of the invention described below, the viral protease is selected from the group consisting of a serine protease, a metalloprotease, an aspartic protease, a cysteine protease, a 3C proteinase, PA transcriptase, adenine protease, 2A protease, chymotrypsin or a trypsin. For example: NS3, NS2, NS-pro cysteine protease, nsP2 cysteine protease, nsP23pro, C protein protease, SFV NS, HIV aspartic protease, nsp4 Arteriviruses protease, HCMV protease. NS2-3, NS3-4Ap protease, HTLV-1 PR.

According to further features in preferred embodiments of the invention described below, the method further comprising the steps of:

(c) designing a plurality of cleavage sequences having the family-wide consensus cleavage sequence; and

(d) identifying in the plurality of cleavage sequences, a cleavage sequence having most rapid cleavage kinetics with the protease.

According to further features in preferred embodiments of the invention described below, the designing comprises designing the cleavage sequences having optimal solubility, temperature sensitivity and/or pH sensitivity.

According to further features in preferred embodiments of the invention described below, the identifying of step (a) comprises empiric experimentation.

According to further features in preferred embodiments of the invention described below, the identifying of step (a) comprises data mining.

According to still a further aspect of the present invention there is provided an isolated peptide comprising an amino acid sequence selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 and 47, the amino acid sequence being no more than 14 amino acids in length and comprises mimetics for inhibiting activity of a respective viral protease.

According to still a further aspect of the present invention there is provided use of the peptide for the manufacture of a medicament identified for treating viral infection.

According to still a further aspect of the present invention there is provided a pharmaceutical composition comprising as an active ingredient the isolated peptide.

The present invention successfully addresses the shortcomings of the presently known configurations by providing compositions for and methods of detecting viruses.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b is a graphic illustration of the plasmids pMND1 (1a) and pMND2 (1b), harboring the HRV 16 3C and SARS 3C1 proteases, respectively.

FIG. 2 is a time course of the expression of recombinant 3C protease from SARS and HRV in E. coli. Transformed clones were induced with 0.4 mM IPTG, and whole cell lysate was analyzed by electrophoresis on a Bioanalyzer 2100 lab-on-chip, using a Protein 200 plus chip kit (Agilent Technologies, Inc, Palo Alto Calif.). Lane 1—MW ladder; Lanes 2-4—pMND-2, at 0, 1, and 6 hours post induction, respectively; Lanes 4-6—pMND-1, 0, 1, and 6 hours post induction, respectively. Note the steady increase of SARS 3CL and HRV 16 3C protease expression with time.

FIG. 3 is a graphic illustration of the superior substrate properties of designed PEP1 substrate, compared to those of the native cleavage site (Ori 2). Reactions contained 5 μM fluorescent substrate and 500 nM recombinant 3C protease, monitored by fluorometry at 340/490±15 nm. Enzyme concentration was 500 pM-1 μM Pep-1-filled ellipses; Ori 2-filled diamonds; Pep1 control-filled rectangles; Ori 2 control-filled triangles. Note superior substrate cleavage with Pep 1.

FIG. 4 is a graphic illustration of the superior reaction kinetics for HRV 3C protease (250 nM) with synthetic Pep 1 substrate, in a range of substrate concentrations from 3 nM to 4 μM.

FIG. 5 is a graphic illustration of the specificity of the synthetic Pep 1 substrate for cleavage with HRV 3C protease. Fluorescent Pep 1 substrate was incubated with E. coli lysate (filled squares), recombinant SARS 3CL lysate (filled circles), recombinant HRV 3C lysate (filled triangles), and control (no lysate) (filled diamonds). Note the absolute specificity of Pep 1 for HRV 3C protease.

FIG. 6 is a graphic illustration of efficient cleavage of Pep 1 substrate by HRV 3C protease under nasal wash conditions. Fluorescent Pep 1 substrate was reacted with recombinant HRV 3C, in the absence (filled squares) and presence (filled circles) of 50% volume nasal wash (sample E 0052). Control was nasal wash (E 0052) (filled diamonds) alone. Note the absence of effect of the nasal wash on substrate cleavage.

FIG. 7 is a scheme of a composition of the present invention which can be used for the detection of cleaving events.

FIG. 8 is a schematic showing the basic events of the system which employs the composition described in FIG. 7 above.

FIG. 9 is a schematic representation showing the separation mechanism of multiple viruses.

FIG. 10 is a schematic representation of the simultaneous detection of three molecules that undergo three types of cleavage reactions.

FIG. 11 is a scheme depicting the sequential proteolysis of Hepatitis C polyprotein by NS3 protease.

### DESCRIPTION OF THE PREFERRED EMBODIMENTS

The present invention is of compositions and methods of using same for the detection of viruses.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The realm of diagnostic assays for detection of acute infections is rapidly changing from antibody detection to pathogen detection, from clinical laboratory based to point-of-care based, from single analyte detection to multiple analyte detection, and is more focused on detection using less invasive approaches for collecting biological samples. New assays are typically more sensitive than are conventional assays and have the capability of providing more information that characterizes the pathogen or the host response to the pathogen. From a public health perspective, the advent of molecular epidemiology, which allows tracking of pathogens based on unique genetic sequences or antigenic properties, has revolutionized how epidemiologists investigate and evaluate epidemics and assess endemic diseases. In addition, the use of point-of-care (POC) devices can impact the detection and surveillance of infections and will enhance our ability to accurately identify the causes of illnesses.

Viral detection via the identification of viral enzymatic activity, has been previously suggested, such as in U.S. Pat. No. 4,952,493 and U.S. Pat. Application No. 20050048473 to Dorit Arad. Such diagnostic assays, which are based on enzymatic activity are far superior to molecular assays, such as the Polymerase Chain Reaction (PCR), as the latter are cumbersome, taking a few hours to perform, and costly. In addition, currently available PCR methods give 40% of false positive and negative results (since they identify also inactive viruses) rendering the method ineffective and also unsafe. Generally, detection of viral protease in a live sample indicates the presence of a live and active viral infection in the body. This is in sharp contrast to PCR analyses which may detect DNA or RNA sequences which are unrelated to the live virus, and antibody detection assays which measure the presence of the body's immune reactivity against the virus. Conversely, antibody assays which detect viral antigens do not always represent the presence of a live virus since viral antigens change and mutate towards specific antibodies even within one disease life cycle.

While conceiving the present invention, the present inventors have hypothesized that the optimal substrate for a protease enzymatic activity assay would be the consensus cleavage site of the polyprotein (i.e., the protease natural substrate) which cleavage kinetics is the most rapid. Substrates which conform to this sequence may be adventitiously used in enzymatic diagnostic tests for the rapid and broad identification of as many viruses which share the same protease.

As is illustrated hereinbelow (and exemplified in details in Example 1 for Hepatitis C NS3 protease), the present inventors identified such kinetically optimized substrates for a large number of virus families (i.e., DNA and RNA viruses, see Example 2). Substrates of HRV and Enterovirus, thus identified, were used for successfully detecting the respective viruses in nasal and cerebral-spinal fluid (CSF) samples and compared to RT-PCR analysis (see Examples 4 and 5). However, as mentioned, the accuracy of RT-PCR detection is dubious, while the protease detection of the present invention has distinct advantages over detection by RT-PCR, as described hereinabove.

Thus, according to one aspect of the present invention there is provided a method for designing a kinetically optimal substrate for a protease of a virus.

As used herein the phrase “kinetically optimal substrate” refers to a conserved amino acid sequence corresponding to a natural cleavage site which is most rapidly cleaved by the protease (defined by K(1M), k(cat) and k(cat)/K(m) assuming the enzyme obeys a Michaelis-Menten kinetic).

The substrate may be an amino acid substrate (i.e., comprising an amino acid sequence) or mimetics thereof.

As used herein the phrase “a protease of a virus” refers to a virally encoded protease (examples of proteases are provided below). The virus may be any virus which expresses a proteolytic enzyme (preferably not exhibiting cleavage specificity of a host protease).

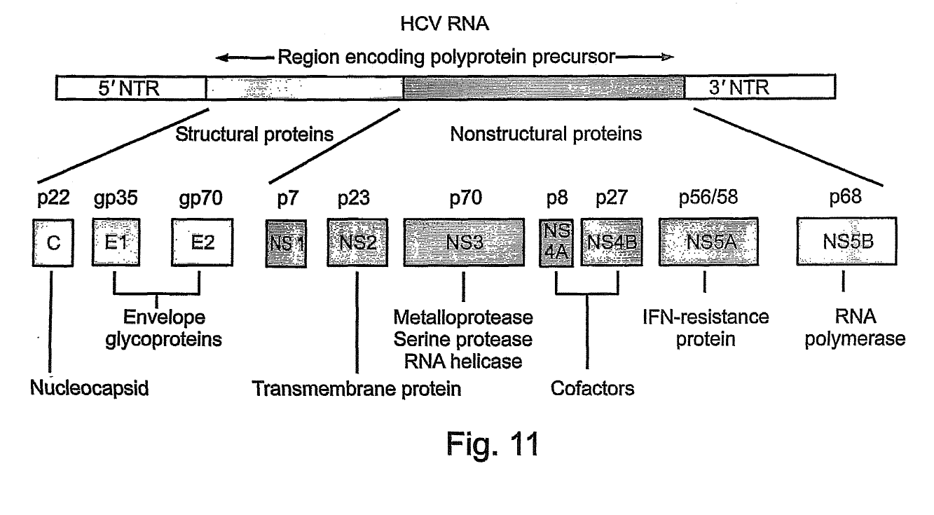

The method of this aspect of the present invention is effected by (a) identifying in a plurality of cleavage sequences of a polyprotein (see e.g., FIG. 11) of at least one strain of the virus, a cleavage sequence displaying most rapid cleavage kinetics by the protease, and (b) identifying a family-wide consensus cleavage sequence displaying most rapid cleavage kinetics, said family-wide consensus cleavage sequence being useful for designing the kinetically optimal substrate for the protease of the virus.

Empiric kinetic characterization may be effected using any method known in the art, typically involving the preparation of soluble, fluorogenic substrates by using recombinant or synthetic methods (e.g., HPLC-based assay). Alternatively, cellular libraries of peptide substrates may be used [e.g., see Boulware and Daugherty 2006 PNAS 103:20-7583:7588]. See also Orr D. C. et al. “Hydrolysis of a series of synthetic peptide substrates by the human rhinovirus 14 3C proteinase, cloned and expressed in E. coli”, J. Gen. Vir. Vol. 70, pp. 2931-42 (1989), the contents of which are incorporated by reference.

Alternatively or additionally literature data mining may be employed for elucidating the cleavage sequence displaying the most rapid cleavage kinetics, as described in details in Example 1 of the Examples section which follows.

Once the cleavage sequence displaying the most rapid cleavage kinetics is at hand, a family-wide consensus cleavage sequence displaying the most rapid cleavage kinetics is identified.

As used herein “a family-wide consensus” refers to the most commonly occurring amino acid at each position of the aligned series of the sequences corresponding to the most rapid cleavage sequence of polyproteins belonging to the same viral family.

This is done by using common bioinformatic tools employing a sequence alignment algorithm such as BLAST (Basic Local Alignment Search Tool, available through www.ncbi.nlm.nih.gov/BLAST) or the Smith-Waterman algorithm (see Example 1 of the Examples section which follows).

Once the family-wide consensus is identified, peptides which comprise this consensus may be designed and their sequences adapted, if needed, to exhibit a biochemical property of interest. Examples include optimal solubility, temperature stability and pH stability. These peptides are considered as the substrates of the present invention.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2—NH, CH2—S, CH2—S═O, O═C—NH, CH2—O, CH2—CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)—CO—), ester bonds (—C(R)H—C—O—O—C(R)—N—), ketomethylene bonds (—CO—CH2—), α-aza bonds (—NH—N(R)—CO—), wherein R is any allyl, e.g., methyl, carba bonds (—CH2—NH—), hydroxyethylene bonds (—CH(OH)—CH2—), thioamide bonds (—CS—NH—), olefinic double bonds (—CH═CH—), retro amide bonds (—NH—CO—), peptide derivatives (—N(R)—CH2—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, Tic, naphthylalanine (Nal), phenylisoserine, threoninol, ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodemosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (e.g., synthetic, Table 2) which can be used with the present invention.

TABLE 1

| | Three-Letter | |
|---|---|---|
| Amino Acid | Abbreviation | One-letter Symbol |
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

Since the present peptides are preferably utilized in the clinic, which requires the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention may be synthesized by any techniques that are known to those skilled in the art of peptide synthesis. For solid phase peptide synthesis, a summary of the many techniques may be found in: Stewart, J. M. and Young, J. D. (1963), “Solid Phase Peptide Synthesis,” W. H. Freeman Co. (San Francisco); and Meienhofer, J (1973). “Hormonal Proteins and Peptides,” vol. 2, p. 46, Academic Press (New York). For a review of classical solution synthesis, see Schroder, G. and Lupke, K. (1965). The Peptides, vol. 1, Academic Press (New York).

In general, peptide synthesis methods comprise the sequential addition of one or more amino acids or suitably protected amino acids to a growing peptide chain. Normally, either the amino or the carboxyl group of the first amino acid is protected by a suitable protecting group. The protected or modified amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth; traditionally this process is accompanied by wash steps as well. After all of the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide, and so forth.

Further description of peptide synthesis is disclosed in U.S. Pat. No. 6,472,505. A preferred method of preparing the peptide compounds of the present invention involves solid-phase peptide synthesis, utilizing a solid support. Large-scale peptide synthesis is described by Andersson Biopolymers 2000, 55(3), 227-50.

Using the above teachings it is possible to identify substrates which can be ultimately used for the detection of any virus and as such may be used in a myriad of research and clinical applications.

Examples of optimal substrates identified according to the present teachings, are provided in Example 2 of the Examples section which follows (e.g., SEQ ID NOs.: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47).

Preferably peptides identified according to the teachings of the present invention are designed no more than 20, preferably 19, preferably 18, preferably 17, preferably 16, preferably 15, preferably 14, preferably 13, preferably 12, preferably 11 preferably 10, preferably 9, preferably 8, preferably 7, preferably 6, preferably 5, preferably 4, preferably 3 amino acids in length.

The present invention is not intended to encompass any of the peptides disclosed and claimed in U.S. Pat. Application 20050048473, and they are specifically excluded from the present invention, as are any known peptides according to the principles disclosed herein.

Peptides of the present invention may be comprised in compositions which further comprise means for cleavage detection (such means are further described hereinbelow, e.g., detectable moiety (also referred to herein as signaling moiety and quencher moiety) and a separating moiety.

Thus, according to another aspect of the present invention there is provided a method of detecting at least one virus in a sample.

Viruses which can be detected in accordance with this aspect of the present invention are those which comprise a protease which cleavage activity can serve as a basis for detection (are not expressed by the host cell). A non-limiting list of proteases which activity can be detected in accordance with this aspect of the present invention include, but are not limited to, a serine protease, a metalloprotease, an aspartic protease, a cysteine protease, a 3C proteinase, PA transcriptase, adenine protease, 2A protease, chymotrypsin or a trypsin. For example: NS3, NS2, NS-pro cysteine protease, nsP2 cysteine protease, nsP23pro, C protein protease, SFV NS, HIV aspartic protease, nsp4 Arteriviruses protease, HCMV protease, NS2-3, NS3-4Ap protease, HTLV-1 PR.

A non limiting list of viruses which can be detected in according with this aspect of the present invention are provided in Example 2 of the Examples section which follows.

As used herein the term “detecting” refers to identifying presence of the virus, classifying the virus and diagnosing a medical condition associated with the virus.

As used herein “diagnosing” refers to classifying a medical condition, determining a severity of such a disease, monitoring disease progression, forecasting an outcome of a disease and/or prospects of recovery.

Thus, for detecting adenovirus the substrate may comprise SEQ ID NO: 1 or 2.

For detecting alphavirus the substrate comprises SEQ ID NO: 3.

For detecting Rubella virus, the substrate comprises SEQ ID NO: 4.

For detecting HIV the substrate comprises SEQ ID NO: 5.

For detecting HTLV the substrate comprises SEQ ID NO: 6, 7 or 8.

For detecting Arteri virus the substrate comprises SEQ ID NO: 9.

For detecting Corona virus the substrate comprises SEQ ID NO: 10.

For detecting SARS corona virus the substrate comprises SEQ ID NO: 11 or 12.

For detecting Torovirus virus the substrate comprises SEQ ID NO: 13.

For detecting CMV virus the substrate comprises SEQ ID NO: 14 or 15.

For detecting Herpes virus the substrate comprises SEQ ID NO: 16.

For detecting Flavivirus virus the substrate comprises SEQ ID NO: 17.

For detecting Denguevirus the substrate comprises SEQ ID NO: 18, 19 or 20.

For detecting West Nile virus the substrate comprises SEQ ID NO: 21, 22 or 23.

For detecting Yellow fever virus the substrate comprises SEQ ID NO: 24, 25 or 26.

For detecting Japanese Encephalitis virus the substrate comprises SEQ ID NO: 27, 28 or 29.

For detecting Tick bone virus the substrate comprises SEQ ID NO: 30, 31 or 32.

For detecting Hepatitis C virus the substrate comprises SEQ ID NO: 33, 34 or 35.

For detecting Pestivirus the substrate comprises SEQ ID NO: 36,

For detecting Hepatitis A virus the substrate comprises SEQ ID NO: 37 or 38.

For detecting HRV the substrate comprises SEQ ID NO: 39 or 40.

For detecting Enterovirus the substrate comprises SEQ ID NO: 41, 42, 43, 44, 45, 46 or 47.

It will be appreciated that if different viruses have similar amino acid sequences, a single protease may cleave the polyprotein of these viruses. Hence, the present invention takes advantage of this specificity to provide detection methods that are specific for a single virus type or for more than one virus type. It will be further appreciated that the present method may also be designed for the identification of non-family related viruses, such as viruses which cause the same symptoms (multiple virus detection) an embodiment which is further described hereinbelow and in Examples 3 and 6 which follows.

As used herein the term “sample” refers to any biological sample (e.g., tissue culture sample or body fluid/tissue sample) which may comprise or permissive for the virus. Preferably the biological sample refers to body fluids such as whole blood, serum, plasma, cerebrospinal fluid, urine, lymph fluids, various external secretions of the respiratory (e.g., nasal wash sample), intestinal, and genitourinary tracts, tears, saliva, semen, sweat, feces, and milk, as well as white blood cells, malignant tissues, amniotic fluid, and chorionic villi.

The method of this aspect of the present invention is effected by contacting the sample with any of the substrates described herein under conditions allowing cleavage of said substrate; and monitoring cleavage of said substrate, wherein the cleavage of said substrate is indicative of the presence of said at least one virus in said sample.

Any assay known in the art for monitoring proteolytic substrate cleavage can be used in accordance with this aspect of the present invention.

Preferably the substrate cleavage sequence is comprised in a composition which further provides means for detection e.g., at least one detectable moiety. Examples are described at length in U.S. Pat. Appl. No. 20050048473 to Dorit Arad which is fully incorporated herein by reference.

As used herein a detectable moiety refers to any molecule which can be directly (e.g., fluorescent, radioisotope) or indirectly (e.g., a pre-enzyme) detected (visualized, counted etc.).

Thus monitoring proteolytic cleavage can be effected by a homogenous assay for the detection of a virus. The chosen substrate is synthesized and linked to a signaling moiety at one side of the cleavage region and to a quencher moiety at the other side of the cleavage region. It will be appreciated that of needed (pending on the configuration of decetion) all the moieties may be placed on one end of the cleavage sequence.

As used herein the phrase “homogeneous assay” refers to an assay not requiring separation of signaling moiety from other assay components.

For the purposes of the invention, a “quencher moiety” is any substance that is capable of reducing or eliminating the signal emitted by the signaling moiety. For example, the quencher moiety may act by absorption of the signal emitted by the signaling moiety or by an energy transfer mechanism. The distance between the signaling moiety and the quencher moiety is such that presence of the quencher moiety substantially reduces or eliminates the signal emitted from the signaling moiety unless the substrate is cleaved at a position resulting in separation of the signaling and quencher moieties.

In one embodiment, the signaling moiety and quencher moiety are separated by no more than 3 or 5 amino acid residues. In another embodiment, the signaling moiety and quencher moiety are separated by no more than 10 amino acid residues. In yet another embodiment, the signaling moiety and quencher moiety are separated by no more than 15 amino acid residues. In yet another embodiment, the signaling moiety and quencher moiety are separated by no more than 20 amino acid residues. Other moieties which are used as means for detection (e.g., also in heterogeneous assays) such as further described hereinbelow may be conjugated according to these guidelines. Also, it will be appreciated that any of the detection means (e.g., moieties) may be conjugated directly or indirectly to the substrate either sequentially or by amino acid modification to any one of the amino acids of the peptide cleavage sequence itself.

The composition is contacted with the sample being tested for the presence of a virus. If the virus is present in the sample, the viral protease is also present. This protease cleaves the substrate and a change in the signal from the signaling moiety can be observed. Such homogenous fluorescent and calorimetric assays are known to those skilled in the art. See, for example: Biochemistry, Allinger, Wang Q. M. et al., “A continuous calorimetric assay for rhinovirus-14 3C protease using peptide p-nitroanilides as substrates” Anal. Biochem. Vol. 252, pp. 238-45 (1997), and Basak S. et al. “In vitro elucidation of substrate specificity and bioassay of proprotein convertase 4 using intramolecularly quenched fluorogenic peptides” Biochem. J. Vol. 380, pp. 505-14 (2004).

In another embodiment of the present invention, the signaling moiety is a chemiluminescent signaling moiety. The chemiluminescent signaling moiety is attached to one side of the cleavage region of the substrate and a fluorescent accepting quencher moiety is attached at the other side of the cleavage region. U.S. Pat. No. 6,243,980, the contents of which are incorporated by reference, describes such a detection system, involving the use of a chemiluminescent 1,2-dioxetane compound as the signaling moiety. If the viral protease is not present in the sample, cleavage of the substrate does not occur. The energy from the 1,2-dioxetane decomposition is transferred to the fluorescent accepting moiety and released at a wavelength distinct from the emission spectrum of the 1,2-dioxetane. If the substrate is cleaved, the fluorescent accepting moiety is separated from the 1,2-dioxetane and a chemiluminescent emission from the dioxetane compound is observed.

In another embodiment, the signaling moiety is a fluorescent compound and the quencher moiety is a fluorescent compound having an excitation spectrum that overlaps the emission spectrum of the signaling moiety. Here, the two moieties are separated apart at a distance consistent with fluorescent resonance energy transfer so that the fluorescent moiety is capable of acting as a resonance energy donor.

In another embodiment, a quenching group, such as a non-fluorescent absorbing dye is used in place of the fluorescent accepting quenching moiety. Suitable quenching groups are described in U.S. Pat. No. 6,243,980, the contents of which are incorporated by reference.

Using such a detection method, the test sample is contacted with the substrate under conditions that allow cleavage of the substrate by the protease if the virus is present in the sample. In one embodiment, the temperature is controlled. For example, the temperature can be controlled at 37° C. to provide optimal conditions for the enzyme reaction. The signal from the cleaved substrate fragment is then detected using a detection device appropriate to the label used.

Another embodiment of the present invention provides a heterogeneous assay for the detection of a virus. A “heterogeneous assay” is an assay in which the solid-phase is separated from another assay component during the assay. A thorough description and examples of heterogeneous assays are provided in Example 6 of the Examples section which follows.

In this case the substrate is comprised in a composition which may have the following general formula.

X-Y-Z

wherein:

Y comprises the substrate of the viral protease, cleavage of X-Y-Z by said viral protease forming cleavage products X-Y′ and Y″-Z wherein Y′ is a first cleavage product of Y and Y′ is a second cleavage product of Y;

X comprises a detectable moiety; and

Z comprises a separating moiety capable of binding to a separate phase of a two phase separating system;

wherein said X-Y-Z does not form a contiguous portion of a natural substrate said viral protease.

The detectable moiety X may directly or indirectly detected and may comprise a labeling agent such as an enzyme, a fluorophore, a chromophore, a protein (e.g., an epitope tag), a chemiluminescent substance and a radioisotope.

Separating moiety Z is being capable of directly or indercslt bind to a separate phase of a two phase separating system (e.g., solid phase and liquid phase). Examples for separating moiety Z include an immunological binding agent, a magnetic binding moiety, a peptide binding moiety, an affinity binding moiety, a nucleic acid moiety. Further examples are provided in Example 6 which follows.

The composition of the present invention may be incubated with the separating system prior to, concomitantly with or following incubation with the sample.

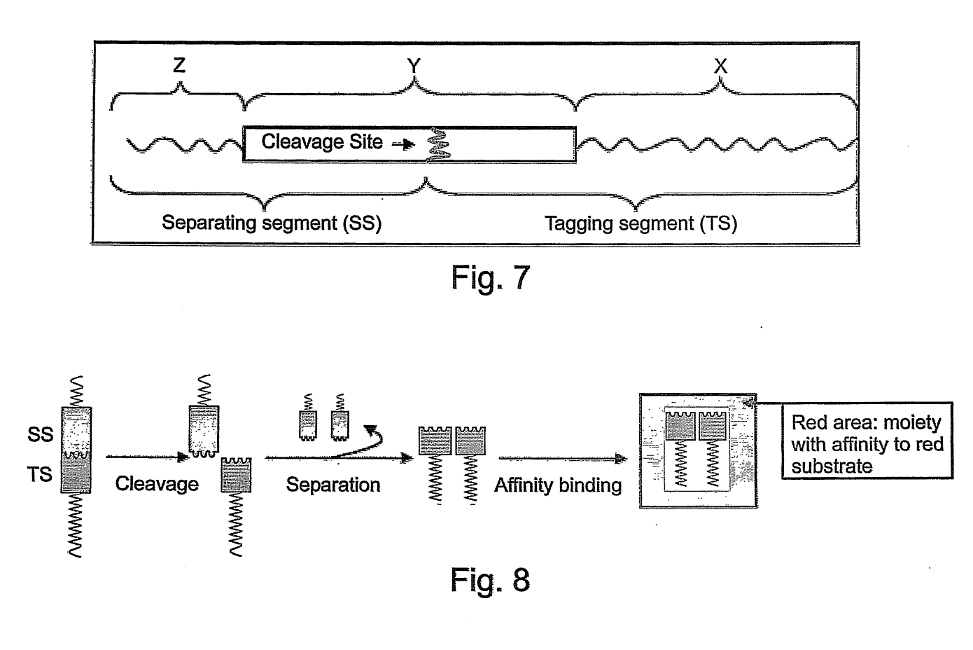

Monitoring cleavage using the heterogeneous assay of the present invention may be effected using any of the embodiments of Example 6 which are schematically depicted in FIGS. 7-10.

Measures should be taken that the detectable moiety does not bind to the separating moiety.

In one embodiment a detactable moiety of the present invention is a pre-enzyme. Accordingly upon substrate cleavage the enzyme can be activated and detected (via the detection of a catalytic activity of same). An example of such a pre-enzyme is pro-Thrombin (factor II) or other enzymes in this cascade.

In any of the embodiments described herein, any of the moieties can be directly linked to the peptide by a covalent bond or indirectly via a spacer molecule having coupling functional groups at each end. Examples of such linkers include an alkyl, a glycol, an ether, a polyether, a polynucleotide and a polypeptide molecule.

Solid-phases suitable for use in the heterogeneous assay include, but are not limited to test tubes, microtiter plates, microtiter wells, beads, dipsticks, polymer microparticles, magnetic microparticles, nitrocellulose, chip arrays and other solid phases familiar to those skilled in the art. The signaling moiety used in the heterogeneous assay may be any label known to those skilled in the art. Such labels include radioactive, calorimetric, fluorescent and luminescent labels.

A heterogeneous chemiluminescent assay for the detection of proteases is described in U.S. Pat. No. 56,243,980, the contents of which are incorporated by reference. In one embodiment, the homogeneous or heterogeneous assay method of the present invention is automated so that a result can be obtained without the need for medical staff to be exposed to a subject thought to be infected by the viral disease under test. For example, the subject can be tested in a clean room (for example, but not limited to P3 type room). The subject can pick up, or get before entering the room, a diagnostic kit, which can include a solid phase coated with a labeled peptide of the type discussed above. For example, the solid phase can be a tissue which was previously immersed with peptide, or a test stick that can be from the type used to test pregnancy. The subject can supply a sample, such as a saliva sample, at a pre-prepared spot on the solid phase.

The solid phase containing the sample is then incubated to allow the enzymatic reaction to occur. In one embodiment, the reaction temperature in controlled at 37° C. to provide optimal conditions for the enzyme reaction. When the incubation is complete, the sample to be tested can be measured on a spectrophotometer, using a remote control, or a mechanical system operated manually from outside the room. The sample can be tested for a qualitative color or UV detection. After the test the sample can be discarded by an automated system, or a remote operated handle that trashes the sample.

In order to detect the presence of a number of viruses at once (such as for detecting a virus associated with specific symptoms or specific hosts e.g., see Example 3), it is possible to use any of the methods known in the art or the above described methods adapted for detection of multiple viruses.

The following provides non-exhaustive examples of such means.

Microplate—in a X well plate. Each well contains a different and specific peptide sequence corresponding to different viruses. With the addition of the clinical sample, the reaction is monitored using a standard microplate reader at the appropriate wavelengths and records which wells demonstrated enzymatic activity. Since each well contains one specific peptide it is possible to elucidate which viral enzymes are present in the clinical sample according to the data provided by the microplate reader. The presence of the enzymes confirms the presence of the viruses.

Medisel chip technology (Schiffenbauer et al. 2002 Anticancer Res. 22:2663-9)—using Medisel technology it is possible immobilize the specific peptides (corresponding to the viruses of interest) on a chip. With the addition of the clinical sample the reaction is monitored using a laser beam. Since each point on the chip contains one specific peptide it is possible to elucidate which viral enzymes are present in the clinical sample according to the data provided by Medisel device. The presence of the enzymes confirms the presence of the viruses.

Separation on column—Specific peptides (corresponding to the viruses of interest) can be attached to beads from a commercial source with a unique DNA spacer. With the addition of the clinical sample the reaction is carried out. Once cleavage of a specific peptide occurs (by the specific viral enzyme in the clinical sample) the quencher is released and the bead emits fluoresce. The beads are then separated on a column via hybridization to the unique DNA spacer and fluorescent is measured for each type of bead (corresponding to each different virus) using a standard fluorometer at the appropriate wavelengths. Only beads that were cut by the viral enzyme emit fluoresce. It is then possible to elucidate which viral enzymes are present in the clinical sample. The presence of the enzymes confirms the presence of the viruses.

Bead FACS separation—Similar to column separation only, the separation step is done by FACS when each specific peptide is attached to a bead with different color. In this method the spacer can be either DNA or peptide or peptide-mimetic or carbohydrate or any organic moiety spacer [Gonzalez (2005) Clin. Biochem. 38:966-72].

Other methods which can be used in accordance with the present invention are described by Tozzoli et al. (2006) Clin. Chem. Lab Med. 44:837-42; Abreu (2005) Ann. N.Y. Acad. Sci. 1050:357-63; Buliard (2005) Ann. Biol. Clin. (Paris) 63:51-8; Yinnaki (2004) J. Immunoassay Immunochem. 25:345-57; Rouquette (2003) 120:676-81; Toellner (2006) Clinical Chemistry 52:1575-1583; Horejsh (2005) Nucl. Acids Res. 33, each of which is incorporated herein by reference in its entirety.

Peptide cleavage sequences identified according to the teachings of the present invention may be used for the detection of new viral strains. Such as for example detection of the SARS epidemic, which displays homology to the original virus family (like corona). This relies on the fast adaptation of the detection method to new viruses that cause pandemics.

Thus, once the genome of the emergent virus is identified and its reproduction system known, viral proteases and those regions of the viral polyprotein that are cleaved by such proteases can be determined by examining the sequence homology between the sequence of the emergent virus and that of known viruses. Based on this alignment selection of the optimal cleavage sequence may be effected.

Kits which comprise the peptides of the present invention are also provided. The different kit components may be packaged in separate containers and admixed immediately before use. Such packaging of the components separately may permit long-term storage without losing the active components' functions. Embodiments in which two or more of components are found in the same container are also contemplated. An exemplary kit may comprise one or more of the following reagents a wash buffer reagent for use using heterogeneous assays; a negative control reagent free of a protease capable of cleaving substrate; a positive control containing a protease capable of cleaving the substrate; (d) a signal generation reagent for development of a detectable signal from the signaling moiety; and (d) a sample collection means such as a syringe, throat swab, or other sample collection device.

For a multiple virus detection kit, in which one or more viruses are being detected as described hereinabove, each multi panel detection kit will be preferably designed according to a common theme, such as different viruses that cause the same or similar diseases, viruses that infect the same tissue or organ, viruses of close phylogenetic relationship such as viruses that are classified to the same family, subfamily and the like, viruses that can be detected in the same body fluid such as saliva, nasal secretion, blood, urine, feces etc., viruses that are common and widespread, viruses that spread via the same body fluid and more.

The reagents included in the kits can be supplied in containers of any sort such that the life of the different components are preserved and are not adsorbed or altered by the materials of the container. For example, sealed glass ampules may contain lyophilized reagents, or buffers that have been packaged under a neutral, non-reacting gas, such as nitrogen. Ampules may consist of any suitable material, such as glass, organic polymers, such as polycarbonate, polystyrene, etc.; ceramic, metal or any other material typically employed to hold similar reagents. Other examples of suitable containers include simple bottles that may be fabricated from similar substances as ampules, and envelopes, that may comprise foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to be mixed. Removable membranes may be glass, plastic, rubber, etc.

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, etc. Detailed instructions may not be physically associated with the kit; instead, a user may be directed to an internet web site specified by the manufacturer or distributor of the kit, or supplied as electronic mail.

The present invention further envisages the use of the peptides of the present invention for the design of therapeutic agents interfering with the virus proteolytic acitivity, and hence with viral replication and infectivity. Preferably, the peptide sequences are modified so as to bind the protease aid inhibit activity of same (e.g., non-reversible inhibitor). As such peptide mimetics may be used to replace the cleavage sequence with a non-cleavable sequence by incorporation of at least one non-peptide bond (as described hereinabove), as long as protease recognition is retained.

Therapeutic peptides of the present invention may be incorporated in a pharmaceutical composition identified for treating a viral disease of interest.

The present invention further envisages screening for anti-viral agents using the compositions of the present invention.

Thus, the present invention provides peptides which may be used in therapeutic and diagnostic applications and compositions and kits which comprise the same.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-m Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I.; ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Hepatitis C NS3 Protease Consensus Cleavage Sequence Identification

The following describes the identification of an optimized cleavage sequence for the NS3 protease of Hepatitis C.

Stage 1: deriving the cleavage mechanism of the polyprotein by its viral protease, essentially the sequence of cleavage events during the life cycle of the virus (1, 4, see FIG. 11 adapted from Hepatitis C Virus (HCV): Models structure and genome organization. Vol 5; Nov. 19, 2003, Cambridge University Press).

Stage 2: comparing all the cleavage sequences of as many strains of the same virus as possible using data bases such as Swiss prot., Pubmed, Gene bank and OWL and aligning them using the FASTA software (see Table 1 below),

TABLE 1

| | | | | cleavage | Seq id |
|---|---|---|---|---|---|
| Cleavage site | protease | strain | Gi: number | sequence | no: |
| NS2<=>NS3 | NS2-3 | HCV | 1814086 | PVSARR\|GKEIF | 52 |
| NS2<=>NS3 | NS2-3 | HCV | 22129793 | PVSARR\|EREIL | 53 |
| NS2<=>NS3 | NS2-3 | HCV | 221607 | QGWRLL\|APITA | 54 |
| NS2<=>NS3 | NS2-3 | HCV | 221615 | PVSARR\|GREIL | 55 |
| NS2<=>NS3 | NS2-3 | HCV | 2764398 | GGWKLL\|APITA | 56 |
| NS2<=>NS3 | NS2-3 | HCV | 59479 | PVSARR\|GREVL | 57 |
| NS2<=>NS3 | NS2-3 | HCV (6a33) | 57791994 | KGWKLL\|APITA | 58 |
| NS2<=>NS3 | NS2-3 | HCV (HC-G9) | 464178 | PVSARL\|GREVL | 59 |
| NS2<=>NS3 | NS2-3 | HCV | 9757542 | PVSARL\|GRELL | 60 |
| | | (JPUT971017) | | | |
| NS2<=>NS3 | NS2-3 | HCV (NZL1) | 1183029 | PVSARL\|GQEVL | 61 |
| NS2<=>NS3 | NS2-3 | HCV (Tr Kj) | 1435035 | QGWRLL\|AHITA | 62 |
| NS2<=>NS3 | NS2-3 | HCV (VAT96) | 6521009 | CLIRLK\|PTLHG | 63 |
| NS2<=>NS3 | NS2-3 | HCV 1b | 5748511 | DLEVVT\|STWVL | 64 |
| C<=>E1 | cellular | HCV | 1381032 | LTIPAS\|AYEVR | 65 |
| C<=>E1 | cellular | HCV | 1814085 | TIPASA\|YEVCN | 66 |
| C<=>E1 | cellular | HCV | 22129793 | TVPASA\|YQVRN | 67 |
| C<=>E1 | cellular | HCV | 221615 | TIPASA\|YQVRN | 68 |
| C<=>E1 | cellular | HCV | 2764398 | TIPASA\|YEVRN | 69 |
| C<=>E1 | cellular | HCV | 59479 | TVPVST\|YEVRN | 70 |
| C<=>E1 | cellular | HCV | 59483 | TVPASA\|YEVRN | 71 |
| C<=>E1 | cellular | HCV | 9843677 | TVPASA\|YHVRN | 72 |
| C<=>E1 | cellular | HCV (6a33) | 57791994 | TTPASA\|LTYGN | 73 |
| C<=>E1 | cellular | HCV (HC-G9) | 464178 | TVPASA\|VGVRN | 74 |
| C<=>E1 | cellular | HCV | 9757542 | TVPVSS\|VEIRN | 75 |
| | | (JPUT971017) | | | |
| C<=>E1 | cellular | HCV (NZL1) | 1183029 | IHPAAS\|LEWRN | 76 |
| C<=>E1 | cellular | HCV (Tr Kj) | 1435035 | TCPASS\|LEYRN | 77 |
| C<=>E1 | cellular | HCV (VAT96) | 6521009 | SVPVSA\|VEVKN | 78 |
| E1<=>E2 | cellular | HCV | 1814085 | FAGVDG\|NTYTT | 79 |
| E1<=>E2 | cellular | HCV | 1814086 | FAGVDG\|NTYVS | 80 |
| E1<=>E2 | cellular | HCV | 1814087 | FVGVDG\|STHVS | 81 |
| E1<=>E2 | cellular | HCV | 1814088 | FAGVDG\|DTYTT | 82 |
| E1<=>E2 | cellular | HCV | 1814089 | FAGVDG\|RTTVT | 83 |
| E1<=>E2 | cellular | HCV | 1814090 | FVGVDG\|STRVS | 84 |
| E1<=>E2 | cellular | HCV | 22129793 | FAGVDA\|ETHVT | 85 |
| E1<=>E2 | cellular | HCV | 221605 | FAGVDG\|ETYTS | 86 |
| E1<=>E2 | cellular | HCV | 221607 | FAGVDG\|ATYTS | 87 |
| E1<=>E2 | cellular | HCV | 2764398 | FAGVDG\|DTHTT | 88 |
| E1<=>E2 | cellular | HCV | 59479 | FAGVDG\|TTYVS | 89 |
| E1<=>E2 | cellular | HCV | 59485 | FAGVDG\|TTTVS | 90 |
| E1<=>E2 | cellular | HCV | 59487 | FAGVDG\|QTRVT | 91 |
| E1<=>E2 | cellular | HCV | 9843677 | FAGVDA\|NTYVT | 92 |
| E1<=>E2 | cellular | HCV (6a33) | 57791994 | FAGVEA\|TTTVG | 93 |
| E1<=>E2 | cellular | HCV (HC-G9) | 464178 | FAGVDA\|ETRVT | 94 |
| E1<=>E2 | cellular | HCV | 9757542 | VAGVDA\|TTYST | 95 |
| | | (JPUT971017) | | | |
| E1<=>E2 | cellular | HCV (NZL1) | 1183029 | FSGVDA\|HTYTT | 96 |
| E1<=>E2 | cellular | HCV (Tr Kj) | 1435035 | FSGVDA\|TTHTT | 97 |
| E1<=>E2 | cellular | HCV (VAT96) | 6521009 | TAGVDA\|QTHTI | 98 |
| E1<=>NS1 | cellular | HCV | 633202 | FSGVDA\|ETYIT | 99 |
| E1<=>NS1 | cellular | HCV | 221615 | ISQAEA\|ALENL | 100 |
| E2<=>NS1 | cellular | HCV | 22129793 | IAQAEA\|ALENL | 101 |
| E2<=>NS1 | cellular | HCV | 2764398 | ISNVEA\|AVERL | 102 |
| E2<=>NS1 | cellular | HCV (6a33) | 57791994 | LGQAEA\|ALEKL | 103 |
| E2<=>NS1 | cellular | HCV | 9757542 | IAQAEA\|TLENL | 104 |
| | | (JPUT971017) | | | |
| E2<=>NS1 | cellular | HCV 2 | 496367 | PQRAYA\|LDTEM | 105 |
| E2<=>NS2 | cellular | HCV | 9843677 | DARVCA\|CLWMM | 106 |
| NS1<=>NS2 | cellular | HCV | 1381032 | PQRAYA\|LDTEV | 107 |
| NS1<=>NS2 | cellular | HCV | 22129793 | PPRAYA\|LDREM | 108 |
| NS1<=>NS2 | cellular | HCV | 2764398 | PHRAYA\|MDNEQ | 109 |
| NS1<=>NS2 | cellular | HCV (6a33) | 57791994 | PQRAYA\|LDQEL | 110 |
| NS1<=>NS2 | cellular | HCV (HC-G9) | 464178 | PQQAYA\|LDAAE | 111 |
| NS1<=>NS2 | cellular | HCV | 9757542 | LLLADA\|RVCVA | 112 |
| | | (JPUT971017) | | | |
| NS1<=>NS2 | cellular | HCV (NZL1) | 1183029 | PPRAYA\|MDREM | 113 |
| NS1<=>NS2 | cellular | HCV 1b | 5420377 | PPQAYA\|MDREM | 114 |
| NS3<=>NS4A | NS3-4Ap | HCV | 221615 | DLEIVT\|STWVL | 115 |
| NS3<=>NS4A | NS3-4Ap | HCV | 2764398 | DLEVIT\|STWVL | 116 |
| NS3<=>NS4A | NS3-4Ap | HCV | 9843677 | DLEVMT\|STWVL | 117 |
| NS3<=>NS4A | NS3-4Ap | HCV (6a33) | 57791994 | DLEVTT\|STWVL | 118 |
| NS3<=>NS4A | NS3-4Ap | HCV | 9757542 | DLEVTT\|SAWVL | 119 |
| | | (JPUT971017) | | | |
| NS3<=>NS4A | NS3-4Ap | HCV (NZL1) | 1183029 | DLEIMT\|STWVL | 120 |
| NS3<=>NS4A | NS3-4Ap | HCV (Tr Kj) | 1435035 | DEMEEC\|SQHLP | 121 |
| NS3<=>NS4A | NS3-4Ap | HCV (VAT96) | 6521009 | AGVAGA\|LVAFK | 122 |
| NS4A<=>NS4B | NS3-4Ap | HCV | 22129793 | DEMEEC\|ASHLP | 123 |
| NS4A<=>NS4B | NS3-4Ap | HCV | 221615 | DEMEEC\|SRHIP | 124 |
| NS4A<=>NS4B | NS3-4Ap | HCV | 2764398 | DEMEEC\|ASKAA | 125 |
| NS4A<=>NS4B | NS3-4Ap | HCV (6a33) | 57791994 | DEMEEC\|SQAAP | 126 |
| NS4A<=>NS4B | NS3-4Ap | HCV | 9757542 | DEMEEC\|ASRAL | 127 |
| | | (JPUT971017) | | | |
| NS4A<=>NS4B | NS3-4Ap | HCV (NZL1) | 1183029 | ECTTPC\|SGSWL | 128 |
| NS4B<=>NS5A | NS3-4Ap | HCV | 221607 | DCSTPC\|SGSWL | 129 |
| NS4B<=>NS5A | NS3-4Ap | HCV | 2764398 | ESTTPC\|SGSWL | 130 |
| NS4B<=>NS5A | NS3-4Ap | HCV | 59479 | DTATPC\|ATSWL | 131 |
| NS4B<=>NS5A | NS3-4Ap | HCV | 9843677 | DCTAPC\|AGSWL | 132 |
| NS4B<=>NS5A | NS3-4Ap | HCV (6a33) | 57791994 | DCPVPC\|SGSWL | 133 |
| NS4B<=>NS5A | NS3-4Ap | HCV (HC-G9) | 464178 | DYPSPC\|SDDWL | 134 |
| NS4B<=>NS5A | NS3-4Ap | HCV | 9757542 | DYPSPC\|NGDWL | 135 |
| | | (JPUT971017) | | | |
| NS4B<=>NS5A | NS3-4Ap | HCV (Tr Kj) | 1435035 | EDVVCC\|SMSYT | 136 |
| NS4B<=>NS5A | NS3-4Ap | HCV (VAT96) | 6521009 | DDVVCC\|SMSYS | 137 |
| NS5A<=>NS5B | NS3-4Ap | HCV | 2764398 | QSVVCC\|SMSYS | 138 |
| NS5A<=>NS5B | NS3-4Ap | HCV (6a33) | 57791994 | GSEVCC\|SMSYS | 139 |
| NS5A<=>NS5B | NS3-4Ap | HCV (NZL1) | 1183029 | DDIVCC\|SMSYT | 140 |
| NS5A<=>NS5B | NS3-4Ap | HCV (VAT96) | 6521009 | EDVVCC\|SMSYS | 141 |
| NS5A<=>NS5B | NS3-4Ap | HCV 2 | 496367 | DSVICC\|SMSYS | 142 |

Step 3: deriving the fastest cleavage sequences according to substitutions or kinetically optimized sites by data mining according to the references listed in Table 2 below. In the case of HCV NS3 protease the site is NS5A/B (3, 4, 5).

TABLE 2

| | |
|---|---|
| 1. | Grakoui, A., D. W. McCourt, C. Wychowski, S. M. Feinstone, and C. M. Rice. |
| | 1993. A second hepatitis C virus-encoded proteinase. Proc. Natl. Acad. Sci. |
| | USA 90: 10583-10587. |
| 2. | Reed KE, Grakoui A, Rice CM. Hepatitis C virus-encoded NS2-3 protease: |
| | cleavage-site mutagenesis and requirements for bimolecular cleavage. |
| 3. | J Virol. 1995 Jul; 69(7): 4127-36 |
| 4. | Bartenschlager, R., L. Ahlborn-Laake, J. Mous, and H. Jacobsen. 1994. Kinetic |
| | and structural analysis of hepatitis C virus polyprotein processing. J. Virol. |
| | 68: 5045-5055. |
| 5. | Failla, C., L. Tomei, and R. De Francesco. 1994. Both NS3 and NS4A are |
| | required for proteolytic processing of hepatitis C virus nonstructural protein. J. |
| | Virol. 68: 3753-3760. |
| 6. | Grakoui, A., C. Wychowski, C. Lin, S. M. Feinstone, and C. M. Rice. 1993. |
| | Expression and identification of hepatitis C virus polyprotein products. J. Virol. |
| | 67: 1385-1395. |
| 7. | Pacini L, Vitelli A, Filocamo G, Bartholomew L, Brunetti M, Tramontano A, |
| | Steinkuhler C, Migliaccio G. In vivo selection of protease cleavage sites by |
| | using chimeric Sindbis virus libraries. J Virol. 2000 Nov; 74(22): 10563-70. |
| 8. | AA Kolykhalov, EV Agapov and CM Rice Specificity of the hepatitis C virus |
| | NS3 serine protease: effects of substitutions at the 3/4A, 4A/4B, 4B/5A, and |
| | 5A/5B cleavage sites on polyprotein processing. J. Virol. 1994 Nov 68(11), |
| | 7525-7533. |
| 9. | Urbani A, Bianchi E, Narjes F, Tramontano A, De Francesco R, Steinkuhler C, |
| | Pessi A. Substrate specificity of the hepatitis C virus serine protease NS3. J Biol |
| | Chem. 1997 Apr 4; 272(14): 9204-9. |

TABLE 3
Activity of substrate peptides based on the sequence of NS4A/B, NS5A/B,
and selected cleavage sites^{a}

| | | Kₘ | | Kₑₐₜ | | Kₑₐₜ/Kₘ | |
|---|---|---|---|---|---|---|---|
| | | (μM) | | (min⁻¹) | | (M⁻¹s⁻¹) | |
| Peptide | Sequence^{b} | + | − | + | − | + | − |
| B5s | BTLEFCSñSY | 27 | 3.5 | 36 | 23 | 22,200 | 109,000 |
| B8s | EQVVRCSñSY | 21 | 7 | 10.5 | 3 | 8,300 | 7,100 |
| B15s | ERVVLCSñSY | 13 | 30 | 44.5 | 30 | 57,000 | 16,600 |
| B19s | ERLVLCSñSY | 12 | 11 | 49 | 30 | 68,000 | 45,000 |
| B28s | ENSVPCSñSY | 56 | 15 | 14.5 | 4 | 4,300 | 4,400 |
| B14s(NS5A/B) | EDVVaCSñSY | 10 | 4 | 47 | 34 | 78,000 | 141,000 |
| NS4A/4Bs | DEMEECASHL | 500 | 10 | 4 | 4 | 133 | 7,000 |

^{a}Kinetic parameters were determined in the presence (+) or absence (−) of 150 mM Nacl.
^{b}ñ, noreleucine.

TABLE 4
Trans

| | | | | | | | | | | | | |
|---|---|---|---|---|---|---|---|---|---|---|---|---|
| Library B | | | | | | | | | | | | |
| B6 | T | P | E | D | T | I | S | C | S | M | S | Y |
| B20 | T | P | E | E | Y | V | N | C | S | M | S | Y |
| B8 | T | P | E | Q | V | V | R | C | S | M | S | Y |
| B14, B46 | T | P | E | D | V | V | C | C | S | M | S | Y |
| B80 | T | P | E | D | V | V | Y | C | S | M | S | Y |
| B62 | T | P | E | D | V | I | Q | C | S | M | S | Y |
| B82 | T | P | E | E | I | V | A | C | S | M | S | Y |
| B10, B27 | T | P | E | L | V | V | P | C | S | M | S | Y |
| B3 | T | P | E | E | V | I | P | C | S | M | S | Y |
| B84 | T | P | E | S | L | V | P | C | S | M | S | Y |
| B28 | T | P | E | N | S | V | P | C | S | M | S | Y |
| B40 | T | P | E | N | M | V | K | C | S | M | S | Y |
| B78 | T | P | E | N | I | V | K | C | S | M | S | Y |
| B44 | T | P | E | M | T | V | K | C | S | M | S | Y |
| B56, B47 | T | P | E | T | T | V | N | C | S | M | S | Y |
| B9 | T | P | E | C | T | V | N | C | S | M | S | Y |
| B74 | T | P | E | S | V | I | N | C | S | M | S | Y |
| B2 | T | P | E | A | L | V | H | C | S | M | S | Y |
| B7 | T | P | E | E | T | V | L | C | S | M | S | Y |
| B1 | T | P | E | E | T | I | L | C | S | M | S | Y |
| B11 | T | P | E | E | A | V | L | C | S | M | S | Y |
| B19, B89 | T | P | E | R | L | V | L | C | S | M | S | Y |
| B81 | T | P | E | R | L | I | L | C | S | M | S | Y |
| B15 | T | P | E | R | V | V | L | C | S | M | S | Y |
| B41 | T | P | E | G | I | V | L | C | S | M | S | Y |
| B42 | T | P | E | V | E | V | L | C | S | M | S | Y |
| B4 | T | P | E | T | V | E | L | C | S | M | S | Y |
| B48 | T | P | E | D | V | E | L | C | S | M | S | Y |
| B43 | T | P | E | D | I | T | L | C | S | M | S | Y |
| B5 | T | P | E | T | L | E | F | C | S | M | S | Y |
| HCV | | | | | | | | | | | | |
| NS4A/4B | E | F | D | E | M | E | E | C | A | S | H | L |
| NS4B/5A | N | E | D | C | S | T | P | C | S | G | S | W |
| NS5A/5B | A | S | E | D | V | V | C | C | S | M | S | Y |

TABLE 5
Cis

| Clone(s) | 8 | 7 | 6 | 5 | 4 | 3 | 2 | 1 | 1′ | 2′ | 3′ | 4′ | 5′ |
|---|---|---|---|---|---|---|---|---|---|---|---|---|---|
| Library A | | | | | | | | | | | | | |
| A35 | T | P | E | R | V | V | L | C | S | A | A | P | |
| A1 | T | P | E | T | I | I | K | C | S | A | A | P | |
| A14 | | T | P | E | V | V | Y | C | S | S | A | A | P |
| Library D | | | | | | | | | | | | | |
| D1 | E | F | D | T | M | S | E | T | A | S | R | G | |
| D3 | E | F | D | E | K | I | Q | T | A | S | R | G | |
| D43 | E | F | D | K | K | L | Q | C | A | S | R | G | |
| D32 | E | F | D | V | R | E | K | C | A | S | R | G | |
| D6 | E | F | D | V | A | T | K | C | A | S | R | G | |
| D48 | E | F | D | S | D | E | K | C | A | S | R | G | |
| D30 | E | F | D | A | T | P | T | C | A | S | R | G | |
| D45 | E | F | D | M | M | T | D | C | A | S | R | G | |
| D7 | E | F | D | S | Q | E | D | C | A | S | R | G | |
| HCV | S | A | D | L | E | V | V | T | S | T | W | V | |
| NS3/4A | | | | | | | | | | | | | |

TABLE 6
Kinetic parameters of cleavage of P6 and P1′-modified decamer peptides
corresponding to the NS4A/NS4B cleavage site
Residues in boldface type indicate modifications with respect to the wild type
sequence. Data are mean ± S.D. of at least three different determinations.

| | NS3 | | | NS3 + Pep4A | | |
|---|---|---|---|---|---|---|
| | Kₘ | k_{cat} | k_{cat}/Kₘ | Kₘ | k_{cat} | k_{cat}/Kₘ |
| Peptides | μM | Min⁻² | M⁻¹s⁻¹ | μM | Min⁻² | M⁻¹s⁻¹ |
| DEMEECASHL | 105 ± 20 | 0.26 ± 0.03 | 47.6 | 42.8 ± 4.1 | 1.4 ± 0.07 | 545 |
| EEMEECASHL | 254 ± 44 | 0.64 ± 0.04 | 42.0 | 166 ± 15 | 2.89 ± 0.20 | 289 |
| NEMEECASHL | 630 ± 62 | 0.37 ± 0.25 | 9.8 | 188 ± 44 | 2.90 ± 0.46 | 257 |
| KEMEECASHL | 1050 ± 430 | 0.26 ± 0.10 | 4.10 | 449 ± 91 | 2.68 ± 0.02 | 99.5 |
| DEMEECSSHL | 273 ± 21 | 0.32 ± 0.01 | 19.5 | 246 ± 54 | 1.66 ± 0.22 | 112 |
| DEMEECFSHL | ND | ND | 0.41 | 156 ± 54 | 0.20 ± 0.02 | 21.3 |

TABLE 7
Residues in boldface type indicate modifications with respect to the wild type
sequence. Data are mean ± S.D. of at least three different determinations.

| | NS3 | | | NS3 + Pep4A | | |
|---|---|---|---|---|---|---|
| | Kₘ | k_{cat} | k_{cat}/Kₘ | Kₘ | k_{cat} | k_{cat}/Kₘ |
| Peptides | μM | Min⁻² | M⁻¹s⁻¹ | μM | Min⁻² | M⁻¹s⁻¹ |
| DEMEECASHL | 105 ± 20 | 0.26 ± 0.03 | 47.6 | 42.8 ± 4.1 | 1.40 ± 0.07 | 545 |
| AEMEECASHL | 125 ± 28 | 0.10 ± 0.02 | 13.3 | 55.0 ± 11 | 0.90 ± 0.06 | 271 |
| DAMEECASHL | 569 ± 150 | 0.86 ± 0.46 | 25.2 | 143 ± 27 | 2.73 ± 0.84 | 317 |
| DEAEECASHL | 409 ± 24 | 0.30 ± 0.02 | 12.2 | 124 ± 22 | 2.13 ± 0.30 | 286 |
| DEMAECASHL | 790 ± 205 | 0.33 ± 0.06 | 7.0 | 190 ± 35 | 2.81 ± 0.66 | 246 |
| DEMEACASHL | 171 ± 23 | 0.26 ± 0.01 | 22.0 | 148 ± 44 | 2.59 ± 0.60 | 292 |
| DEMEEAASHL | | not cleaved | | | not cleaved | |
| DEMEECAAHL | 191 ± 11 | 0.67 ± 0.04 | 58.4 | 144 ± 5.6 | 3.08 ± 0.15 | 356 |
| DEMEECASAL | 231 ± 39 | 0.85 ± 0.16 | 61.5 | 130 ± 15 | 3.61 ± 0.96 | 462 |
| DEMEECASHA | 260 ± 37 | 0.58 ± 0.12 | 37.1 | 219 ± 10 | 3.00 ± 0.94 | 226 |

A Cis-acting protease cleaves only adjacent cleavage sites. A Trans-acting protease acts on remote cleavage sites.

### Cis-

1. P1—has threonine.
2. P4—accepted mostly residues with a non-polar aliphatic side chain.
3. P2—shows a preference to charged residues.
4. The Specificity of the protease cis-cleavage site NS4A/C junction is highly degenerated (6) indicating that the cleavage is determent by polyprotein folding.

### Trans

1. P1—the Cysteine is important and can not be replaced.
2. P3—accepted only valine, glutamic acid, threonine, and isoleucine. Substitutions of Glu to Asn or Lys had no effects
3. P4—tolerated most residues.
4. P2—displayed a preference for leucine. Substitution of Glu to Asn or Lys had no effects.
5. P5—preferred charged residues (negatively charged residue→aspartate).
6. P6—An acidic residue is important. (tyrosine)
7. P3—residues contribute to efficient substrate recognition (gln).
8. P4′—residues contribute to efficient substrate recognition (hydrophobic residues→Leu).
9. P7—Substitutions of Phe to His or Arg. Had no effects.
10. P51—Substitutions of Glu to Lys had no effects.
11. P2′—Substitutions of Gln to Lys had no effect.
12. P1′—Substitutions of Ser to Ile, Thr, Arg, Ala, Asp, or His permitted efficient cleavage. However, substitution with Pro dramatically inhibited cleavage (7)
13. Efficient in vitro cleavage requires a peptide substrate of at least 10 residues spanning P6 to P4′.

Step 4: deriving a consensus sequence site according to the two fastest sites and substitutions from step 3 and generalizing the consensus sequence formula from step 4 according to the allowed variations from step 3. All data combined results, for HCV NS3 protease in the following sequence:

(SEQ ID NO: 33)

(T/S/Acidic)X(E/T/I/N/K/D/V)(C/F)C −/− X(M/norL)
(S/D)(Y/ Hydrophobic)

### Example 2

### Kinetically Optimized Substrates Identified According to the Teachings of the Present Invention

The following symbols were used in the presentation of the consensus sequences.

### Hydrophobic: G, A, V, L, I, M, W, F, Y, H

### Basic: Q, N, K, H, R

HB donor: K, R, S, C, T, Q, N, Y

### Acidic: E, D, Y

### Aromatic: Y, F, H, W

-/-: Cleavage point

### Adenoviridae (1-9):

Simian: adenovirus 25, adenovirus 24, adenovirus 23, adenovirus 22, adenovirus 21, adenovirus 19.
Porcine: adenovirus C, adenovirus B, adenovirus A, adenovirus 5, adenovirus 4, adenovirus 3, adenovirus 2, adenovirus 1.
Ovine: adenovirus B, adenovirus A, adenovirus 5, adenovirus 4, adenovirus 3, adenovirus 2, adenovirus 1.
Murine: adenovirus A, adenovirus 1.
Human: adenovirus F, adenovirus E, adenovirus D, adenovirus C, adenovirus B, adenovirus A, adenovirus 9, adenovirus 8, adenovirus 7, adenovirus 6, adenovirus 51, adenovirus 50, adenovirus 5, adenovirus 49, adenovirus 48, adenovirus 47, adenovirus 46, adenovirus 45, adenovirus 44, adenovirus 43, adenovirus 42, adenovirus 41, adenovirus 40, adenovirus 4, adenovirus 39, adenovirus 38, adenovirus 37, adenovirus 36, adenovirus 35, adenovirus 34, adenovirus 33, adenovirus 32, adenovirus 31, adenovirus 30, adenovirus 3, adenovirus 29, adenovirus 28, adenovirus 27, adenovirus 26, adenovirus 25, adenovirus 24, adenovirus 23, adenovirus 22, adenovirus 21, adenovirus 20, adenovirus 2, adenovirus 19, adenovirus 18, adenovirus 17, adenovirus 16, adenovirus 15, adenovirus 14, adenovirus 13, adenovirus 12, adenovirus 11, adenovirus 10, adenovirus 1
Equine: adenovirus B, adenovirus A, adenovirus 2, adenovirus 1
Canine: adenovirus 2, adenovirus 1, adenovirus.
Bovine: adenovirus C, adenovirus B, adenovirus A, adenovirus 9, adenovirus 3, adenovirus 2, adenovirus 10, adenovirus 1

Adenovirus
Kinetically optimal site: GX/G site

SEQ ID NO: 1

(M/I/L)XGX −/− G

SEQ ID NO: 2

(M/L/I/V/N/Q)X(A/G)X −/− G

### Togaviridae (10-33)

Alphavirus: Aura virus, Barmah Forest virus, Eastern equine encephalitis virus Middelburg virus, Ndumu virus, Bebaru virus, Chikungunya virus, Getah virus, Mayaro virus, O'nyong-nyong virus, Ross River virus, Semliki forest virus, Una virus, Trocara virus, Cabassou virus, Mucambo virus, Pixuna virus, Venezuelan equine encephalitis virus, Fort Morgan virus, Highlands J virus, Sindbis virus, Western equine encephalomyelitis virus, Whataroa virus, Alphavirus HBb17, Norwegian salmonid alphavirus, Rio Negro virus, Seal louse virus.

Alphavirus:

SEQ ID NO: 3

(A/V)G(A/G/Basic) −/− (A/G/Y)(Hydrophobic)

Rubivirus: Rubella virus, Rubella virus (strain BRD 1), Rubella virus (strain BRDII), Rubella virus (strain Cendehill), Rubella virus (strain M33), Rubella virus (strain RN-UK86), Rubella virus (strain THERIEN), Rubella virus (strain TO-336 vaccine), Rubella virus (strain TO-336), Rubella virus (vaccine strain RA27/3).

Rubella virus:

SEQ ID NO: 4

SRGG −/− GTC

### Retroviridae (34-60)

Orthoretrovirinae, Lentivirus, Primate lentivirus group:
Human immunodeficiency virus 1: HIV-1 M:C_{—}92BR025, HIV-1 M:C_ETH2220, HIV-1 M:F1_{—}93BR020, HIV-1 M:F1_VI850, HIV-1 M:F2_MP255C, HIV-1 M:F2_MP257C, HIV-1 M:G_{—}92NG083, HIV-1 M:G_SE6165, HIV-1 M:H_{—}90CF056, HIV-1 M:H_VI991, HIV-1 M:J_SE9173, HIV-1 M:J_SE9280, HIV-1 M:K_{—}96CM-MP535, HIV-1 M:K_{—}97ZR-EQTB11, HIV-1 N_YBF106, HIV-1 N_YBF30, HIV-1 O_ANT70, HIV-1 O_MVP5180, Human immunodeficiency virus 3, Human immunodeficiency virus type 1 (ARV2/SF2 ISOLATE), Human immunodeficiency virus type 1 (BH10 ISOLATE), Human immunodeficiency virus type 1 (BH5 ISOLATE), Human immunodeficiency virus type 1 (BH7 isolate), Human immunodeficiency virus type 1 (BH8 ISOLATE), Human immunodeficiency virus type 1 (BRAIN ISOLATE), Human immunodeficiency virus type 1 (BRU ISOLATE), Human immunodeficiency virus type 1 (CDC-451′ ISOLATE), Human immunodeficiency virus type 1 (CLONE 12), Human immunodeficiency virus type 1 (ELI ISOLATE), Human immunodeficiency virus type 1 (HXB2 ISOLATE), Human immunodeficiency virus type 1 (HXB3 ISOLATE), Human immunodeficiency virus type 1 (isolate YU2), Human immunodeficiency virus type 1 (JH3 ISOLATE), Human immunodeficiency virus type 1 (JRCSF ISOLATE), Human immunodeficiency virus type 1 (KB-1 isolate), Human immunodeficiency virus type 1 (Lai isolate), Human immunodeficiency virus type 1 (MAL ISOLATE), Human immunodeficiency virus type 1 (MFA ISOLATE), Human immunodeficiency virus type 1 (MN ISOLATE), Human immunodeficiency virus type 1 (NDK ISOLATE), Human immunodeficiency virus type 1 (NEW YORK-5 ISOLATE), Human immunodeficiency virus type 1 (NIT-A isolate), Human immunodeficiency virus type 1 (OYI ISOLATE), Human immunodeficiency virus type 1 (PV22 ISOLATE), Human immunodeficiency virus type 1 (RF/HAT ISOLATE), Human immunodeficiency virus type 1 (SC ISOLATE), Human immunodeficiency virus type 1 (SF162 ISOLATE), Human immunodeficiency virus type 1 (SF33 ISOLATE), Human immunodeficiency virus type 1 (STRAIN UGANDAN/ISOLATE U455), Human immunodeficiency virus type I (WMJ1 isolate), Human immunodeficiency virus type 1 (WMJ2 ISOLATE), Human immunodeficiency virus type 1 (Z-84 ISOLATE), Human immunodeficiency virus type 1 (Z2/CDC-Z34 ISOLATE), Human immunodeficiency virus type 1 (ZAIRE 3 ISOLATE), Human immunodeficiency virus type I (ZAIRE 6 ISOLATE), Human immunodeficiency virus type 1 (ZAIRE HZ321 ISOLATE), Human immunodeficiency virus type 1 lw12.3 isolate.
Human immunodeficiency virus 2:Human immunodeficiency virus type 2 (ISOLATE BEN), Human immunodeficiency virus type 2 (ISOLATE ROD), Human immunodeficiency virus type 2 (ISOLATE ST), Human immunodeficiency virus type 2 (isolate ST/24.1C#2), HIV-2 B_EHO, HIV-2 B_UC1, HIV-2.D205, Human immunodeficiency virus type 2 (ISOLATE D205,7), Human immunodeficiency virus type 2 (isolate 7312A), Human immunodeficiency virus type 2 (ISOLATE CAM2), Human immunodeficiency virus type 2 (ISOLATE D194), Human immunodeficiency virus type 2 (ISOLATE GHANA-1), Human immunodeficiency virus type 2 (isolate KR), Human immunodeficiency virus type 2 (ISOLATE NIH-Z), Human immunodeficiency virus type 2 (ISOLATE SBLISY).

HIV

SEQ ID NO: 5

(S/G)(Q/G/R/K)(N/C/D)(Y/Hydrophobic/Aromatic) −/−
P(I/V/Hydrophobic(V/Q)

### Retroviridae; Orthoretrovirinae; Deltaretrovirus; Primate T-lymphotrophic

Human T-cell lymphotrophic virus type 1 (Caribbean isolate), Human T-cell lymphotrophic virus type 1 (isolate MT-2), Human T-cell lymphotrophic virus type 1 (strain ATK), Human T-cell lymphotrophic virus type I (African isolate), Human T-cell lymphotrophic virus type 1 (North American isolate).

| | |
|---|---|
| HTLV(Human T-cell lymphotrophic virus) | |
| Optimal site: CA/NC and Pr/P3 | |
| (V/L/T/P)X(Hydrophobic)(F/L) -/- V(Hydrophobic)Q | SEQ ID NO: 6 |
| KVKV(F/L) -/- VVQPK | SEQ ID NO: 7 |
| PPX(Hydrophobic)L -/- PI | SEQ ID NO; 8 |

### Nidovirales: Arteriviridae (78-82)

Arterivirus: Equine arteritis virus Bucyrus, Lactate dehydrogenase elevating, virus C, Lactate dehydrogenase elevating virus Plagemann, Lelystad virus, PRRSV 16244B, PRRSV HB-1(sh)/2002, PRRSV HB-2(sh)/2002, PRRSV HN1, PRRSV VR2332, Simian hemorrhagic fever virus.

| | |
|---|---|
| Arteri virus: | |
| (Aromatic/Basic/L)E -/- (G/S) | SEQ ID NO: 9 |

### Nidovirales: Coronaviridae (61-77)

Coronavirus: Canine coronavirus, Feline coronavirus, Human coronavirus 229E, Porcine epidemic diarrhea virus, Transmissible gastroenteritis virus, unclassified Bovine coronavirus, Human coronavirus OC43, Murine hepatitis virus, Porcine hemagglutinating encephalomyelitis virus, Puffinosis coronavirus, Rat coronavirus, SARS coronavirus, Infectious bronchitis virus, Turkey coronavirus, Bat coronavirus China 2005, Bat coronavirus strain 61, Bat coronavirus ZS-2005, Bird droppings coronavirus, Chicken enteric coronavirus, Duck coronavirus, Goose coronavirus, Human coronavirus NO, Parrot coronavirus AV71/99, Pigeon coronavirus

| | |
|---|---|
| Coronavirus: | |
| 3C Protease: | |
| Optimal site: P1/P2 | |
| (S/Hydrophobic)XLQ -/- (S/A)GX(Hydrophobic/Basic) | SEQ ID NO: 10 |
| SARS corona virus: | |
| PLpro: | |
| (Hydrophobic)(K/R/N/Q)GG -/- /(A/K)(Hydrophobic) | SEQ ID NO: 11 |
| 3CL: | |
| Optimal site P1/P2 | |
| (A/S)(Hydrophobic)LQ -/- SGF | SEQ ID NO: 12 |

Torovirus: Bovine torovirus, Breda virus, Equine torovirus, Berne virus, Human torovirus, Porcine torovirus, Porcine torovirus (strain P10).

Torovirus:

SEQ ID NO: 13

(Basic)(Aromatic/Basic/P)Q -/- (S/A/G)

### Herpesviridae (83-105);

### Betaherpesvirinae: Cytomegalovirus

Rhesus cytomegalovirus strain 68-1, Human herpesvirus 5 (strain 1042), Human herpesvirus 5 (strain 119), Human herpesvirus 5 (strain 2387) Human herpesvirus 5 (strain 4654), Human herpesvirus 5 (strain 5035), Human herpesvirus 5 (strain 5040), Human herpesvirus 5 (strain 5160), Human herpesvirus 5 (strain 5508), Human herpesvirus 5 strain AD169, Human herpesvirus 5 strain Eisenhardt, Human herpesvirus 5 strain Merlin, Human herpesvirus 5 strain PT, Human herpesvirus 5 strain Toledo, Human herpesvirus 5 strain Towne, Chimpanzee cytomegalovirus, Aotine herpesvirus 1, Baboon cytomegalovirus OCOM4-37, ercocebus agilis cytomegalovirus 1, Cercopithecus cephus cytomegalovirus 1, Colobus badius cytomegalovirus 1, Colobus guereza cytomegalovirus 1, Crocidura russula cytomegalovirus 1, Macaca fascicularis cytomegalovirus 1, Mandrillus cytomegalovirus, Phacochoerus africanus cytomegalovirus 1, Pongo pygmaeus cytomegalovirus 1, Porcine cytomegalovirus, Simian cytomegalovirus,

| | |
|---|---|
| CMV | |
| Kinetically optimal site: M site | |
| VV (X not K)A -/- S | SEQ ID NO: 14 |
| VVNA -/- SCR | SEQ ID NO: 15 |

### Alphaherpesvirinae: Simplexvirus:

Bovine herpesvirus type 2 (strain BHM-1), Bovine herpesvirus type 2 (strain BMV), Cercopithecine herpesvirus 1 (strain E2490), Cercopithecine herpesvirus 16, Cercopithecine herpesvirus 2 (SA8), Herpes simplex virus (type 1/strain 17), Herpes simplex virus (type 1/strain A44), Herpes simplex virus (type 1/strain Angelotti), Herpes simplex virus (type 1/strain CL101), Herpes simplex virus (type 1/strain CVG-2), Herpes simplex virus (type 1/strain F), Herpes simplex virus (type 1/strain HFEM), Herpes simplex virus (type 1/strain HZT), Herpes simplex virus (type 1/strain MGH-10), Herpes simplex virus (type 1/strain MP), Herpes simplex virus (type 1/strain Patton), Herpes simplex virus (type 1/strain R19), Herpes simplex virus (type 1/strain SC16), Herpes simplex virus 1 strain R-15, Human herpesvirus 7 strain JI, Human herpesvirus 1 strain KOS, Human herpesvirus 2 (Herpes simplex virus type 2), Herpes simplex virus (type 2/strain B4327UR), Human herpesvirus 2 strain 186, Human herpesvirus 2 strain 333, Human herpesvirus 2 strain G, Human herpesvirus 2 strain HG52, Human herpesvirus 2 strain SA8, Human herpesvirus 2 strain SN03, Human herpesvirus 2 strain SS01, Human herpesvirus 2 strain ST04.

| | |
|---|---|
| Herpes simplex | |
| Optimal site: C Terminal cleavage | |
| (Hydrophobic/Aromatic)(Hydrophobic)(Q/N/E/D)A -/- S(S/T/D/E) | SEQ ID NO: 16 |
| (Hydrophobic/HB Donor) | |

### Flaviviridae; Flavivirus (106-133)

Alfuy virus, Alkhurma hemorrhagic fever virus, Apoi virus, Aroa virus, Bagaza virus, Banzi virus, Batu Cave virus, Bouboui virus, Bukalasa bat virus, Bussuquara virus, CY1014 virus, Cacipacore virus, Carey Island virus, Cell fusing agent virus, Cowbone Ridge virus, Dakar bat virus, Deer tick virus, Dengue virus, Edge Hill virus, Entebbe bat virus, Flavivirus CbaAr4001, Flavivirus FSME, Flavivirus sp., Gadgets Gully virus, Greek goat encephalitis virus, Iguape virus, Ilheus virus, Israel turkey meningoencephalomyelitis virus, Japanese encephalitis virus, Jugra virus, Jutiapa virus, Kadam virus, Kamiti River virus, Karshi virus, Kedougou virus, Kokobera virus, Koutango virus, Kumlinge virus, Kunjin virus, Kyasanur forest disease virus, Langat virus, Langat virus (strain TP21), Langat virus (strain Yelantsev), Louping ill virus, Meaban virus, Modoc virus, Montana myotis leukoencephalitis virus, Murray Valley encephalitis virus, Naranjal virus, Negishi virus, Ntaya virus, Omsk hemorrhagic fever virus, Phnom Penh bat virus, Potiskum virus, Powassan virus, Rio Bravo virus, Rocio virus, Royal Farm virus, Russian Spring-Summer encephalitis virus, Saboya virus, Sal Vieja virus, San Perlita virus, Saumarez Reef virus, Sepik virus, Sitiawan virus, Sokoluk virus, Spanish sheep encephalitis virus, Spondweni virus, St. Louis encephalitis virus, Stratford virus, Tamana bat virus, Tembusu virus, Tick-borne encephalitis virus, Tick-borne flavivirus, Tick-borne powassan virus (strain 1b), Turkish sheep encephalitis virus, Tyuleniy virus, Uganda S virus, Usutu virus, Wang Thong virus, Wesselsbron virus, West Nile virus, Yaounde virus, Yellow fever virus, Yokose virus, Zika virus, mosquito-borne viruses.

| | |
|---|---|
| Flavivirus | |
| Optimal site: NS3/4A and NS2A/2B | |
| (Basic/G)(Basic)(Basic) -/- (S/G/A) | SEQ ID NO: 17 |
| Denguevirus: | |
| G(Basic)(Basic) -/- (S/T/A)(Hydrophobic)(HB donor) | SEQ ID NO: 18 |
| (S/T/A)XGRK -/- S(Hydrophobic)T | SEQ ID NO: 19 |
| AAGRK -/- SLT | SEQ ID NO: 20 |
| West Nile virus: | |
| (HB donor/A)X(Basic)(Basic) -/- (S/G)X(Hydrophobic) | SEQ ID NO: 21 |
| PNRKR -/- GWPA | SEQ ID NO: 22 |
| FASGKR -/- SQIG V 23 | |
| Yellow fever virus: | |
| G(Basic)(Basic) -/- (S/G)X(Hydrophobic) | SEQ ID NO: 24 |
| FGRR -/- SIP | SEQ ID NO: 25 |
| EGRR -/- GAA | SEQ ID NO: 26 |
| Japanese Encephalitis virus: | |
| (Basic)(Basic) -/- (S/G)(Hydrophobic)(Hydrophobic) | SEQ ID NO: 27 |
| NKKR -/- GWPA | SEQ ID NO: 28 |
| AATGKR -/- SA(Hydrophobic) | SEQ ID NO: 29 |
| Tick bone virus: | |
| (Basic)(Basic) -/- S(Hydrophobic)X(Acidic) | SEQ ID NO: 30 |
| RGRR -/- SFSE V | SEQ ID NO: 31 |
| SGRR -/- SFGD | SEQ ID NO: 32 |

### Flaviviridae; Hepacivirus (134-142); Hepatitis C Virus

Hepatitis C virus subtype 1a, Hepatitis C virus subtype 1b, Hepatitis C virus subtype 1c, Hepatitis C virus subtype 1d, Hepatitis C virus subtype 1e, Hepatitis C virus subtype 1f, Hepatitis C virus subtype 2a, Hepatitis C virus subtype 2b, Hepatitis C virus subtype 2c, Hepatitis C virus subtype 2d, Hepatitis C virus subtype 2f, Hepatitis C virus subtype 2i, Hepatitis C virus subtype 2k, Hepatitis C virus subtype 3a, Hepatitis C virus subtype 3b, Hepatitis C virus subtype 3g, Hepatitis C virus subtype 3k, Hepatitis C virus subtype 4a, Hepatitis C virus subtype 4c, Hepatitis C virus subtype 4d, Hepatitis C virus subtype 4f, Hepatitis C virus subtype 4h, Hepatitis C virus subtype 4k, Hepatitis C virus subtype 5a, Hepatitis C virus subtype 6a, Hepatitis C virus subtype 6b, Hepatitis C virus subtype 6d, Hepatitis C virus subtype 6g, Hepatitis C virus subtype 6h, Hepatitis C virus subtype 6k, Hepatitis C virus (isolate EC1), Hepatitis C virus (isolate EC10), Hepatitis C virus (isolate Glasgow), Hepatitis C virus (isolate HC-J2), Hepatitis C virus (isolate HC-J5), Hepatitis C virus (isolate HC-J7), Hepatitis C virus (isolate HCT18), Hepatitis C virus (isolate HCT27), Hepatitis C virus (isolate HCV-476), Hepatitis C virus (isolate HCV-KF), Hepatitis C virus (isolate Hunan), Hepatitis C virus (isolate TH), Hepatitis C virus (isolate VT204), Hepatitis C virus (isolate VT316), Hepatitis C virus (isolate VT681), Hepatitis C virus (isolate VT886), Hepatitis C virus (isolate VT887), Hepatitis C virus (isolate VT897), Hepatitis C virus (isolate VT898),

| | |
|---|---|
| Hepatitis C | |
| NS3 Protease: | |
| Optimal site: NS5A/B | |
| (T/S/Acidic)X(E/T/I/N/K/D/V)(C/P)C -/- X(M/norL)(S/D)(Y/Hydrophobic) | SEQ ID NO: 33 |
| NS2-3 Protease: | |
| (Hydrophobic)XXX(not E or P) -/- (Hydrophobic/Basic)X(not P) | SEQ ID NO: 34 |
| GXXLL -/- (A/V/R)PI | SEQ ID NO: 35 |

### Flaviviridae; Pestivirus (143-144);

Border disease virus, Bovine viral diarrhea virus, Chamois pestivirus, Classical Swine Fever virus, Classical swine fever virus, Hog cholera virus, Ovine pestivirus, Pestivirus, Porcine pestivirus, Pronghorn antelope pestivirus,

| | |
|---|---|
| Pestivirus: | |
| Optimal site: 3/4A and 4B/5A | |
| (Hydrophobic)X(G/N/Q)L -/- (S/A)(HB donor/G/A)(N/A) | SEQ ID NO: 36 |

### Picornaviridae; Hepatovirus; Hepatitis A Virus (145-151):

Hepatitis A virus (STRAIN 18F), Hepatitis A virus (STRAIN 24A), Hepatitis A virus (STRAIN 43C), Hepatitis A virus (STRAIN CR326), Hepatitis A virus (strain GA76), Hepatitis A virus (STRAIN HM-175), Hepatitis A virus (STRAIN LA), Hepatitis A virus (STRAIN LCDC-1), Hepatitis A virus (STRAIN MBB), Hepatitis A virus (strain MSM1), Simian hepatitis A virus (strain AGM-27), Simian hepatitis A virus (strain CY-145).

| | |
|---|---|
| Hepatitis A | |
| 3C protease: | |
| Kinetically optinial site: 2C/3A | |
| (L/I)WSQ -/- G/S(D/E)D | SEQ ID NO: 37 |
| EFFQ -/- SEP | SEQ ID NO: 38 |

### Picornaviridae; Rhinovirus (152-163);

Human rhinovirus 10, Human rhinovirus 100, Human rhinovirus 11, Human rhinovirus 12, Human rhinovirus 13, Human rhinovirus 15, Human rhinovirus 16, Human rhinovirus 18, Human rhinovirus 19, Human rhinovirus 1A, Human rhinovirus 1B, Human rhinovirus 2, Human rhinovirus 20, Human rhinovirus 21, Human rhinovirus 22, Human rhinovirus 23, Human rhinovirus 24, Human rhinovirus 25, Human rhinovirus 28, Human rhinovirus 29, Human rhinovirus 30, Human rhinovirus 31, Human rhinovirus 32, Human rhinovirus 33, Human rhinovirus 34, Human rhinovirus 36, Human rhinovirus 38, Human rhinovirus 39, Human rhinovirus 40, Human rhinovirus 41, Human rhinovirus 43, Human rhinovirus 44, Human rhinovirus 45, Human rhinovirus 46, Human rhinovirus 47, Human rhinovirus 49, Human rhinovirus 50, Human rhinovirus 51, Human rhinovirus 53, Human rhinovirus 54, Human rhinovirus 55, Human rhinovirus 56, Human rhinovirus 57, Human rhinovirus 58, Human rhinovirus 59, Human rhinovirus 60, Human rhinovirus 61, Human rhinovirus 62, Human rhinovirus 63, Human rhinovirus 64, Human rhinovirus 65, Human rhinovirus 66, Human rhinovirus 67, Human rhinovirus 68, Human rhinovirus 7, Human rhinovirus 71, Human rhinovirus 73, Human rhinovirus 74, Human rhinovirus 75, Human rhinovirus 76, Human rhinovirus 77, Human rhinovirus 78, Human rhinovirus 8, Human rhinovirus 80, Human rhinovirus 81, Human rhinovirus 82, Human rhinovirus 85, Human rhinovirus 88, Human rhinovirus 89, Human rhinovirus 9, Human rhinovirus 90, Human rhinovirus 94, Human rhinovirus 95, Human rhinovirus 96, Human rhinovirus 98, Human rhinovirus B, Human rhinovirus 14, Human rhinovirus 17, Human rhinovirus 26, Human rhinovirus 27, Human rhinovirus 3, Human rhinovirus 8001 Finland November 1995, Human rhinovirus 35, Human rhinovirus 37, Human rhinovirus 6253 Finland September 1994, Human rhinovirus 9166 Finland September 1995, Human rhinovirus 4, Human rhinovirus 42, Human rhinovirus 9864 Finland September 1996, Human rhinovirus 5, Human rhinovirus 52, Human rhinovirus 7425 Finland December 1995, Human rhinovirus 5928 Finland May1995, Human rhinovirus 70, Human rhinovirus 72, Human rhinovirus 79, Human rhinovirus 83, Human rhinovirus 8317 Finland August 1996, Human rhinovirus 86, Human rhinovirus 7851 Finland September 1996, Human rhinovirus 92, Human rhinovirus 93, Human rhinovirus 97, Human rhinovirus 99, Antwerp rhinovirus 98/99, Human rhinovirus 263 Berlin 2004, Human rhinovirus strain Hanks, Untyped human rhinovirus OK88-8162, Amblyomma americanum, Human rhinovirus UC.

| | |
|---|---|
| HRV | |
| 3C protease: | |
| Kinetically optimal site: 2C/3A | |
| FQ -/- GP | SEQ ID NO:39 |
| 2A protease: | |
| Kinetically optimal site: VP1/2A | |
| (S/T)(Hydrophobic) -/- G(Hydrophobic) | SEQ ID NO: 40 |

### Picornaviridae; Enterovirus (164-179)

Bovine enterovirus, Bovine enterovirus strain K2577, Bovine enterovirus strain SL305, Bovine enterovirus type 2, Coxsackievirus A16, Coxsackievirus B3, Enterovirus A01-2A-1, Enterovirus H02-2A-3, Enterovirus H02-2B-1, Enterovirus H04-2B-2, Enterovirus Hu, Enterovirus I01-1-2, Enterovirus S01-2A-1, Enterovirus S02-1-6, Enterovirus S03-1-3, Enterovirus S06-1-1, Human coxsackievirus A16, Human coxsackievirus A9, Human coxsackievirus A9B, Coxsackievirus, Enterovirus 69, Enterovirus 74, Enterovirus 79, Enterovirus 81, Enterovirus 82, Enterovirus 83, Enterovirus 86, Enterovirus Yanbian 96-83csf, Enterovirus Yanbian 96-85csf, Human coxsackievirus A1, Human coxsackievirus A10, Human coxsackievirus A11, Human coxsackievirus A12, Human coxsackievirus A13, Human coxsackievirus A14, Human coxsackievirus A15, Human coxsackievirus A17, Human coxsackievirus A18, Human coxsackievirus A19, Human coxsackievirus A2, Human coxsackievirus A20, Human coxsackievirus A21 Human coxsackievirus A22, Human coxsackievirus A24, Human coxsackievirus A3, Human coxsackievirus A4, Human coxsackievirus A5, Human coxsackievirus A6, Human coxsackievirus A7, Human coxsackievirus A8, Human coxsackievirus B1, Human coxsackievirus B2, Human coxsackievirus B4, Human coxsackievirus B5, Human coxsackievirus B6, Human echovirus 1, Human echovirus 11, Human echovirus 12, Human echovirus 13, Human echovirus 14, Human echovirus 15, Human echovirus 16, Human echovirus 17, Human echovirus 18, Human echovirus 19, Human echovirus 2, Human echovirus 20, Human echovirus 21, Human echovirus 24, Human echovirus 25, Human echovirus 26, Human echovirus 27, Human echovirus 29, Human echovirus 3, Human echovirus 30, Human echovirus 31, Human echovirus 32, Human echovirus 33, Human echovirus 4, Human echovirus 5, Human echovirus 6, Human echovirus 7, Human echovirus 8, Human echovirus 9, Human enterovirus 68, Human enterovirus 69, Human enterovirus 70, Human enterovirus 71, Human enterovirus 73, Human enterovirus 74, Human enterovirus 75, Human enterovirus 77, Human enterovirus 78, Human enterovirus 89, Human enterovirus 90, Human enterovirus 91, Human enterovirus B, Human poliovirus 1, Human poliovirus 2, Human poliovirus 3, Porcine enterovirus 10, Porcine enterovirus 9, Porcine enterovirus J10, Porcine enterovirus J4, Porcine enterovirus J6, Simian picornavirus 7, Simian picornavirus 7′, Swine vesicular disease virus, Wild poliovirus type 3.

| | |
|---|---|
| Enterovirus cleavage sites | |
| 3C protease: | |
| Kinetically optimal site: 2C3A | |
| FQ -/- GP | SEQ ID NO:41 |
| 2A protease: | |
| Kinetically optimal site: VPI2A | |
| Coxsackievirus: | |
| TT -/- GXX(G/S)QQ | SEQ ID NO: 42 |
| A(Aromatic) -/- G(H/Q)Q(G/S) | SEQ ID NO: 43 |
| Echovirus: | |
| (T/N)T -/- GX(Aromatic)(G/S)QQ | SEQ ID NO: 44 |
| (T/N)(Y/H) -/- GAF(G/S)QQ | SEQ ID NO: 45 |
| (Small Hydrophobic)F -/- GQQ(G/S) | SEQ ID NO: 46 |
| (T/E)X -/- GXF(G/S)QQS | SEQ ID NO: 47 |

Data mining for the sequences provided herein is based on the literature references placed at the end of this document.

### Example 3

### Exemplary Multipanel Detection Kits

TABLE 8
Respiratory Kit:

Virus strains
Corona Viruses
SARS
Other corona viruses
HMPV (Human Meta pneumo virus)
Influenza A + B
Avian Influenza
Adeno virus
RSV (Respiratory Syncytial Virus)
Rhino virus
Para influenza viruses:
1, 2, 3
Special Respiratory Kit
Hanta virus
La Crosse Encephalitis

TABLE 9
Gastro-intestinal Kit:
Virus strains

Rota virus
Adeno 40/41
Hepatitis A
Hepatitis C
Hepatitis E
caliciviruses
CMV (Cytomegalovirus)

TABLE 10
Meningitis Kit:

Virus strains
Enteroviruses (1-~80)
[including polio virus 1,2,3)
West Nile virus
Herpes Simplex 1 & 2 & 6
Special Meningitis Kit
Toga viruses (Eastern/Western Equine
Flavi viruse (St. Luis Encephalitis, Japanese Encephalitis . . . )
Rabies

TABLE 11
Sexually transmitted disease Kit:
Virus strains

HIV strains
Herpes simplex 1
Herpes simplex 2
HSV-1
HSV-2
HPV (Human Papilloma Viruses)
HTLV-1

TABLE 12
Travelers Kit:
Virus strains

Hepatitis A
Hepatitis B
Hepatitis C
HIV
Herpes Virus 1 & 2

TABLE 13
Veterinarian Kit:
Virus strains

Rabies
Distemper

The following provides example of diseases which can be detected using the above-mentioned kits.

Respiratory kit:

whooping cough, unspecified organism
pneumococcus infect.in condition classif.elsewhere;unsp.site
adenovirus inf.in conditions classif.elsewhere,unsp.site
ac.alcoholic intoxic.in alcoholism, episodic drinking behavior
acute nonsuppurative otitis media, unspecified
acute serous otitis media
acute mucoid otitis media
acute sanguinous otitis media
acute allergic serous otitis media
acute allergic mucoid otitis media
acute allergic sanguinous otitis media
ac.suppurat.otitis media without spontan.rupture of eardrum
acute suppurat.otitis media with spontan.rupture of eardrum
chronic tubotympanic suppurative otitis media
unspecified chronic suppurative otitis media
unspecified suppurative otitis media
unspecified otitis media
acute mastoiditis without complications
subperiosteal abscess of mastoid
acute mastoiditis with other complications
perforation of tympanic membrane, unspecified
central perforation of tympanic membrane
attic perforation of tympanic membrane
other marginal perforation of tympanic membrane
multiple perforations of tympanic membrane
total perforation of tympanic membrane
atrophic flaccid tympanic membrane
atrophic nonflaccid tympanic membrane
unspecified disorder of tympanic membrane
acute nasopharyngitis (common cold)
acute maxillary sinusitis
acute frontal sinusitis
acute ethmoidal sinusitis
acute sphenoidal sinusitis
other acute sinusitis
acute sinusitis, unspecified
acute pharyngitis
acute tonsillitis
acute laryngitis
acute tracheitis without mention of obstruction
acute tracheitis with obstruction
acute laryngotracheitis without mention of obstruction
acute laryngotracheitis with obstruction
acute epiglottitis without mention of obstruction
acute epiglottitis with obstruction
croup
acute laryngopharyngitis
acute upper respiratory infections of other multiple sites
acute upper respiratory infections of unspecified site
acute bronchitis
acute bronchiolitis
pneumonia due to adenovirus
pneumonia due to respiratory syncytial virus
pneumonia due to parainfluenza virus
pneumonia due to other virus not elsewhere classified
viral pneumonia, unspecified
pneumococcal pneumonia
pneumonia due to klebsiella pneumoniae
pneumonia due to pseudomonas
pneumonia due to hemophilus influenzae (h. influenzae)
bacterial pneumonia, unspecified
pneumonia in cytomegalic inclusion disease
pneumonia in whooping cough
pneumonia in anthrax
pneumonia in aspergillosis
pneumonia in other systemic mycoses
pneumonia in other infectious diseases classified elsewhere
bronchopneumonia, organism unspecified
pneumonia, organism unspecified
influenza with pneumonia
influenza with other respiratory manifestations
influenza with other manifestations
bronchitis, not specified as acute or chronic
other chronic bronchitis
extrinsic asthma without mention of status asthmaticus
extrinsic asthma with status asthmaticus
intrinsic asthma without mention of status asthmaticus
intrinsic asthma with status asthmaticus
asthma,unspecified type,without mention of status asthmaticus
asthma, unspecified type, with status asthmaticus
pneumonitis due to inhalation (food, vomitus, or n.o.s.)
empyema with fistula
empyema without mention of fistula
pleurisy without mention of effusion or current tuberculosis
pleurisy with effusion,with a bacterial cause other than tbc
other specified forms of pleural effusion, except tuberculous
unspecified pleural effusion
abscess of lung
abscess of mediastinum
pulmonary collapse
respiratory failure
other diseases of respiratory system, not elsewhere classified
unspecified disease of respiratory system
toxic gastroenteritis and colitis
other and unspec.noninfectious gastroenteritis and colitis
cyanosis
respiratory abnormality, unspecified
hyperventilation
orthopnea
other dyspnea and respiratory abnormality
cough
asphyxia

gastro-enteritis kit:

cholera due to vibrio cholerae
cholera due to vibrio cholerae el tor
cholera, unspecified
typhoid fever
paratyphoid fever a
paratyphoid fever b
paratyphoid fever c
paratyphoid fever, unspecified
salmonella gastroenteritis
salmonella septicemia
localized salmonella infection, unspecified
salmonella meningitis
salmonella pneumonia
salmonella arthritis
salmonella osteomyelitis
other localized salmonella infections
other specified salmonella infections
salmonella infection, unspecified
shigella dysenteriae
shigella flexneri
shigella boydii
shigella sonnei
other specified shigella infections
shigellosis, unspecified
staphylococcal food poisoning
botulism
food poisoning due to clostridium perfringens (c. welchii)
food poisoning due to other clostridia
food poisoning due to vibrio parahaemolyticus
food poisoning, unspecified
acute amebic dysentery without mention of abscess
chronic intestinal amebiasis without mention of abscess
amebic nondysenteric colitis
amebic liver abscess
amebic lung abscess
amebic brain abscess
amebic skin ulceration
amebic infection of other sites
amebiasis, unspecified
balantidiasis
giardiasis
coccidiosis
intestinal trichomoniasis
other specified protozoal intestinal diseases
unspecified protozoal intestinal disease
intestinal infection due to arizona group of paracolon
bacilli
intestinal infection due to aerobacter aerogenes
intestinal infection due to proteus (mirabilis) (morganii)
intestinal infection due to staphylococcus
intestinal infection due to pseudomonas
intestinal infection due to other specified bacteria
bacterial enteritis, unspecified
intestinal infection due to other organism, not elsew.class.
infectious colitis, enteritis, and gastroenteritis
colitis, enteritis, gastroenteritis presumed infectious origin
infectious diarrhea
diarrhea of presumed infectious origin

### Meningitis Kit

unspecified bacteremia

bacteremia

septicemia

pneumococcal septicemia

bacterial meningitis

pneumococcal meningitis

meningitis in other bacterial diseases classified elsewhere

meningitis due to other specified bacteria

meningitis, unspecified

septic arthritis

periorbital cellulitis

mastoiditis and related conditions

acute mastoiditis

acute mastoiditis without complications

pneumococcal peritonitis

herpetic meningoencephalitis

herpetic septicemia

### Sexually Transmitted Diseases (STD) Kit

herpes simplex

herpes simplex dermatitis of eyelid

herpes simplex disciform keratitis

herpes simplex iridocyclitis

herpes simplex meningitis

herpes simplex otitis externa

herpes simplex with ophthalmic complications

herpes simplex with other ophthalmic complications

herpes simplex with other specified complications

herpes simplex with other specified complications

herpes simplex with unspecified complication

herpes simplex with unspecified ophthalmic complication

herpes simplex without mention of complication

herpes zoster

herpes zoster dermatitis of eyelid

herpes zoster iridocyclitis

herpes zoster keratoconjunctivitis

herpes zoster with meningitis

herpes zoster with ophthalmic complications

herpes zoster with other nervous system complications

herpes zoster with other nervous system complications

herpes zoster with other ophthalmic complications

herpes zoster with other specified complications

herpes zoster with other specified complications

herpes zoster with unspecified complication

herpes zoster with unspecified nervous system complication

herpes zoster without mention of complication

herpetic gingivostomatitis

herpetic infection of penis

herpetic meningoencephalitis

herpetic septicemia

herpetic ulceration of vulva

herpetic vulvovaginitis

herpetic whitlow

### Example 4

### Protease Cloning and Activity Assays

Materials and Experimental Methods

Protease Assay:

HRV3C: A typical in vitro assay of HRV 3C activity was performed at room temperature in 25 mM HEPES, 150 mM NaCl, 1 mM EDTA, 5% Glycerol, PH 7.5 and total volume=1 ml. HRV 3C protease activity was monitored in samples, in cell lysates, in transformed cell extract (recombinant) and in samples of purified, recombinant HRV16 3C. A fluorescent substrate (pep 1, 5, 6, 8, Ori 2; EDANS/DANCYL) was typically used at concentration of 4 μM. Detectable substrates include, but are not limited to, Ori 2, PEP1. Reactions were monitored using a fluorometer at excitation/emission 340/490±15 nm corresponding to the excitation/emission characteristics of the EDANS/DABCYL groups. Enzyme concentration varied from 1 μM to 500 μM.

Alternatively, 3C protease assays were performed using a substrate labeled with 7-methoxy coumarin-4-acetic acid (MOC) fluorochrome and dinitrophenol (DNP) quencher in 100 μl volumes in a 96 well format at 30° C., containing 25 mM Tris HCl pH 8.0, 150 mM NaCl, 1 mM EDTA pH 8.0, 6 mM DTT, 2-6 uM substrate, 2% DMSO, 416 nM 3C protease and inhibitor as needed. Fluorescence is monitored by excitation at 328 nm and emission at 393 nm with 10 nm cutoffs. Data were analyzed with the nonlinear regression analysis program EnzFitter (BioSoft) with the equation:

Kᵢ=(I/((Vₘₐₓ×S)/V₀)/Kₛ)−I−S

Substrate concentrations used are lower than the Kₘ of the substrate (16.8 uM) so no corrections for an S/Kₘ term were used.

Additions to the reaction included DTT, Glycerol, Na₂SO₄, BSA and nasal wash fluid.

Enterovirus (Non-Polio Enterovirus NPEV) Activity Assay: Fresh CSF samples (stored 1-2 day at 4° C.) were sonicated for 3×20 sec at 4° C. 100 μl of the lysate was added to a 100 μl of 2× reaction buffer using 4 μM of Pep 1 as substrate. The reaction was performed at room temperature and was monitored using a fluorometer at excitation/emission 340/490±15 nm, respectively.

Tissue Culture Assay: H1 HeLa cells were grown in DMEM medium supplemented with 5% FBS, 1% pen-strep and 1% nonessential amino acids. Confluent flasks of H1 HeLa cells were infected with HRV sterotype14 & 1A at MOI of 1-10 PFU/cell. Cells were harvested 48h post infection either by trypsin treatment or scraping. Cells were washed 3 times with PBS and re-suspended in reaction buffer. Cells were broken by sonication 3×15 sec on ice followed by centrifugation 10 min, 13000 rpm, 4° C. The cleared lysate (contain the soluble 3C pro) was used for the assay.

Cloning of SARS and HRV 16 3C proteases: SARS and HRV proteases were cloned as a source of proteases for the experiments and kits.

HRV 16: Primers were designed to 4320-4869 bp position in the genome. The forward primer was added with 5′ prime extension, CACC, to facilitate directional cloning into a topoisomerase cloning vector. The reverse primer was used to introduce a stop codon (TGA) to the 3′ prime end. The expected PCR product using these primers is 556 bp. The template used was originated from a vector containing HRV 16 cDNA (GenBank Accession No. gi: 3915817)

| | |
|---|---|
| HRV 16 fwd: | |
| 5′ CACCGGTCCAGAAGAAGAAT 3′ | SEQ ID NO: 48 |
| HRV 16 rev: | |
| 5′ TCATTGTTGTTCAGTGAAGTAT 3′ | SEQ ID NO: 49 |

SARS: Primers were designed to 9985-10902 bp position in the genome (SARS 3CL, GenBank Accession No. gi:37999886). The forward primer was added with 5′ prime extension, CACC, to facilitate directional cloning. The reverse primer was used to introduce a stop codon (TAA) to the 3′ prime end. The PCR product expected using these primers is 925 bp. The template used originated from a cDNA library obtained from Dr. Av-Gay (Av Gay; Vancouver, Canada)

| | |
|---|---|
| SARS fwd: | |
| 5′ CACCAGTGGTTTTAGGAAAATGGC 3′ | SEQ ID NO: 50 |
| SARS rev: | |
| 5′ TTATTGGAAGGTAACACCAGAGC 3′ | SEQ ID NO: 51 |

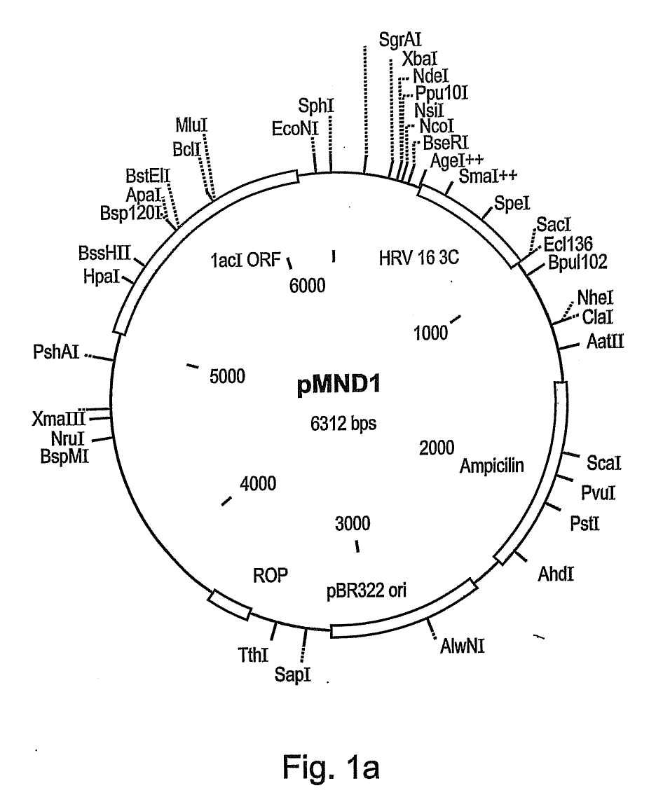

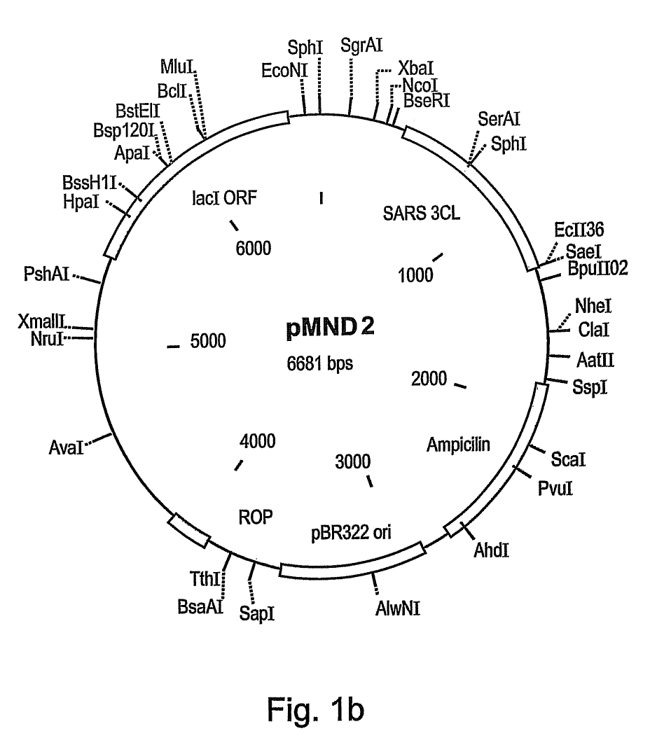

PCR amplification of the genes was performed using proof reading polymerase (Pfu) in a standard reaction mix (1× reaction buffer, 0.5 mM dNTPs, 100 pmol primers and 1 unit pfu. The following cycling protocol was used—3 min at 94° C. followed by 35 cycles of 94° C. 1 min, 59° C. 1 min, 72° C. 1 min and finished with 10 min at 72° C.). The PCR products from both SARS 3CL and HRV 16 3C proteases were cloned directly into pET 151/D-TOPO (Invitrogen, Carlsbad Calif.) (FIGS. 1a and 1b). Out of 35 colonies screened for HRV 16 3C protease one was found to be positive. For SARS 3CL protease 3 out of 15 screened were found to be positive.

Experimental Results

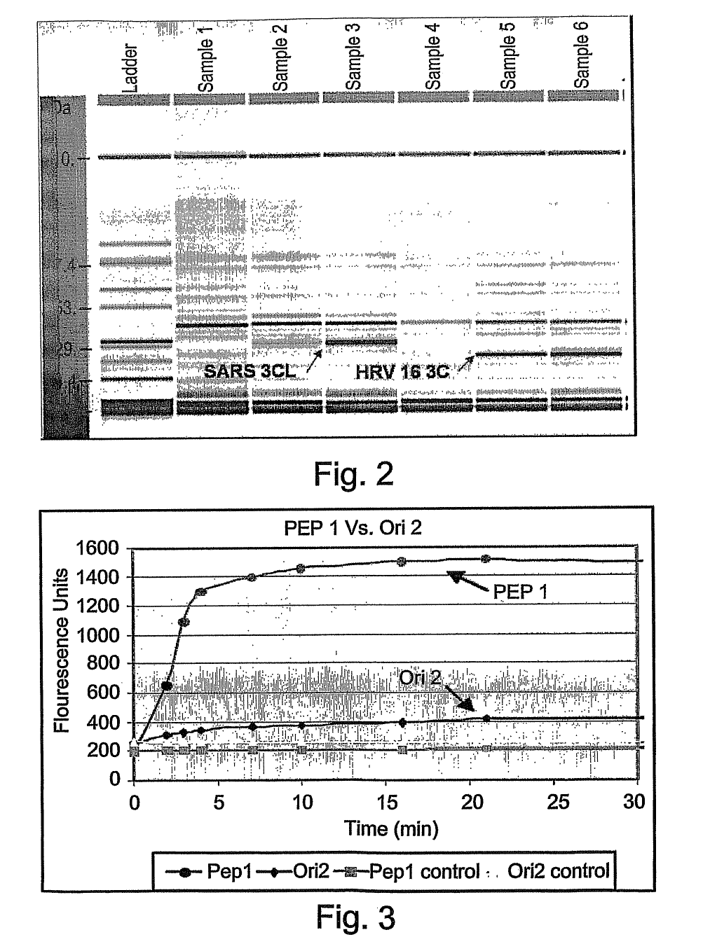

Cloning of SARS and HRV 16 3C proteases: SARS 3CL and HRV 16 proteases were cloned into pET 151/D-TOPO (Invitrogen, Carlsbad Calif.) as a source of proteases for the experiments and kits, as described hereinabove. FIGS. 1a and 1b show the respective SARS 3CL (pMND2) and HRV 16 3C (pMND1) plasmids. Out of 35 colonies screened for HRV 16 3C protease one was found to be positive. For SARS 3CL protease 3 out of 15 screened were found to be positive. Recombinant HRV 16 3C and SARS 3CL protease activity was detected in whole cell lysate from the transformed bacteria (FIG. 2).

Superior Substrate Activity Using Optimal Cleavage Sequence Substrate Peptides—In Vitro Assay

Peptide design: In order to provide selective, optimally efficient substrates for detection and characterization of proteases of interest, cleavage of native and designed peptide substrates is compared in an in-vitro assay. Peptide substrates were designed for use in assay for HRV 3C proteases. Peptide substrate sequences were designed either according to the native cleavage site sequence, or selected according to the method of the present invention, Typically, substrate sequence design was determined by executing a multiple sequence alignment of a plurality of known HRV cleavage sites, and determining the most optimal amino acid at a specific position based on its bioinformatics properties. Table 14 below illustrates the 3C protease substrates used in the in-vitro assay, and their origin.

TABLE 14

| SEQ ID | | | |
|---|---|---|---|
| NO | Name | Sequence | Target Design |
| 143 | Ori 2 | (DABCYL)-S-A-I-F-Q-G-P-I-S-M-D | Based on an original cleavage site of |
| | | (EDANS)-K | HRV 16. |
| 144 | PEP1 | (DABCYL)-L-E-A-L-F-Q-G-P-D | Designed based on multiple HRVs |
| | | (EDANS)-S-Q | cleavage sites and bioinformatics. |
| 145 | PEP3 | E-A-L-F-Q-pNA | Based on HRVs cleavage sites and |
| | | PEP 1 | |
| 146 | PEP4 | D-S-L-E-V-L-F-Q-pNA | Based on HRVs cleavage sites. |
| 147 | PEP5 | (DABCYL)-L-E-V-L-F-Q-G-P-D | Designed based on multiple HRVs |
| | | (EDANS)-S-Q | cleavage sites and bioinformatics. |
| 148 | PBP6 | (DABCYL)-T-S-A-V-L-Q-S-G-F-R-D | Based on an original cleavage site of |
| | | (EDANS)-K | SARS. |
| 149 | PEP7 | T-S-A-V-L-Q-pNA | Based on an original cleavage site of |
| | | SARS. | |
| 150 | PEP8 | (DABCYL)-L-E-A-L-F-Q-A-A-D | Designed not to be cut by HRVs |
| | | (EDANS)-S-Q-NH₂ | proteases. Also has a further N |
| | | | terminus modification designed to |
| | | | block the terminus and reduce |
| | | | background. |
| 151 | PEP9 | (DABCYL)-L-E-A-L-F-Q-G-P-D | Designed based on multiple HRVs |
| | | (EDANS)-S-Q-NH₂ | cleavage sites and bioinformatics. |
| | | | Also has a further N terminus |
| | | | modification designed to block the |
| | | | terminus and reduce background. |

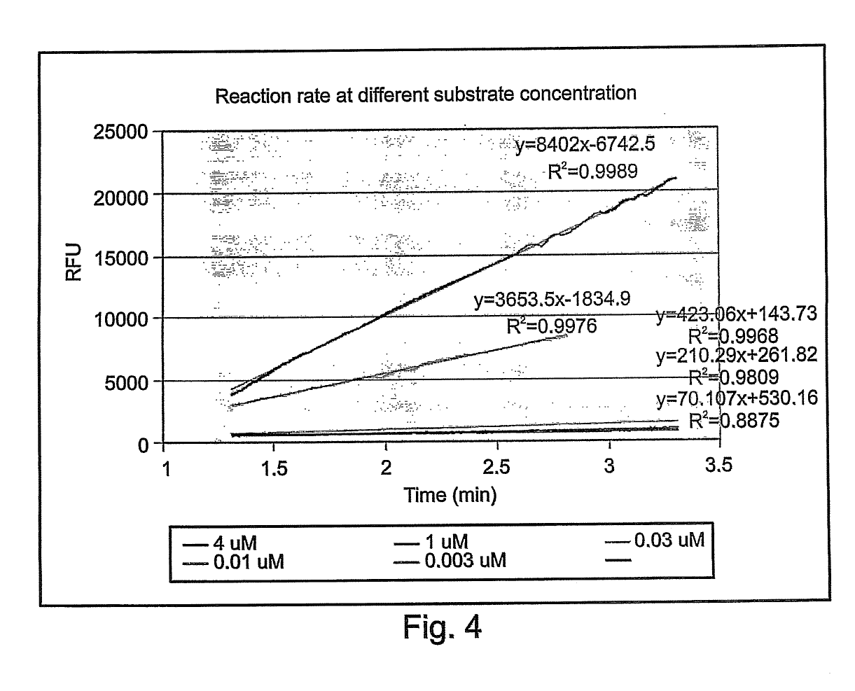

Determining optimal peptide substrate for 3C protease: In order to determine the most optimal cleavage substrate for 3C protease, cleavage of the synthetic peptide substrates Pep1-Pep9 (see Table 14 above) by recombinant 3C protease (see Example 1 hereinabove) was compared with cleavage of the native substrate sequence Ori 2. The substrate exhibiting the most rapid kinetics out of Pep 1-Pep 9 was Pep1, designed on the basis of multiple HRV 3C cleavage sites and bioinformatics. FIG. 3 illustrates the superior kinetics of the 3C protease assay, when compared with Ori 2. FIG. 4 illustrates the kinetics of cleavage of Pep1 by recombinant 3C protease in a range of substrate concentrations from 0.003 μM to 4 μM, using 250 nM 3C protease. Linear kinetics over at least 3 minutes reaction was observed for all substrate concentrations assayed.

Determining optimal conditions for 3C protease assay using optimal peptide substrate: In order to establish the optimal conditions for 3C protease assay using the designed substrate, sensitivity to alteration of reaction conditions was assessed. Concentrations of significant components of the standard reaction buffer, such as DTT, glycerol, Na₂SO₄ and BSA were altered, and the effect on reaction rate (RFU/min) was determined. The results indicated that the optimal reaction buffer for use with the Pep1 substrate comprises 6 mM DTT, 5% glycerol, 0.8 M Na₂SO₄, and 0.1-1 mg BSA/ml.

Determining sensitivity of 3C protease assay using optimal peptide substrate: Concentrations of the enzyme in clinical samples is expected to often be low. In order to determine the lower limits of detection of 3C protease activity using the designed peptide substrate, 4 μM of Pep1 was assayed with a range of recombinant enzyme concentrations. The lower limit of protease detection in this assay was determined to be from 0.5-1.0 nM 3C protease.

Determining specificity of 3C protease assay using optimal peptide substrate: Specificity of the designed substrate for the desired cleavage activity is crucial for evaluation of cleavage activity in an actual clinical sample. In order to test for specificity of HRV 3C protease for Pep1, enzymatic cross-reactivity of Pep1 substrate with SARS proteases and E. coli lysates was tested.

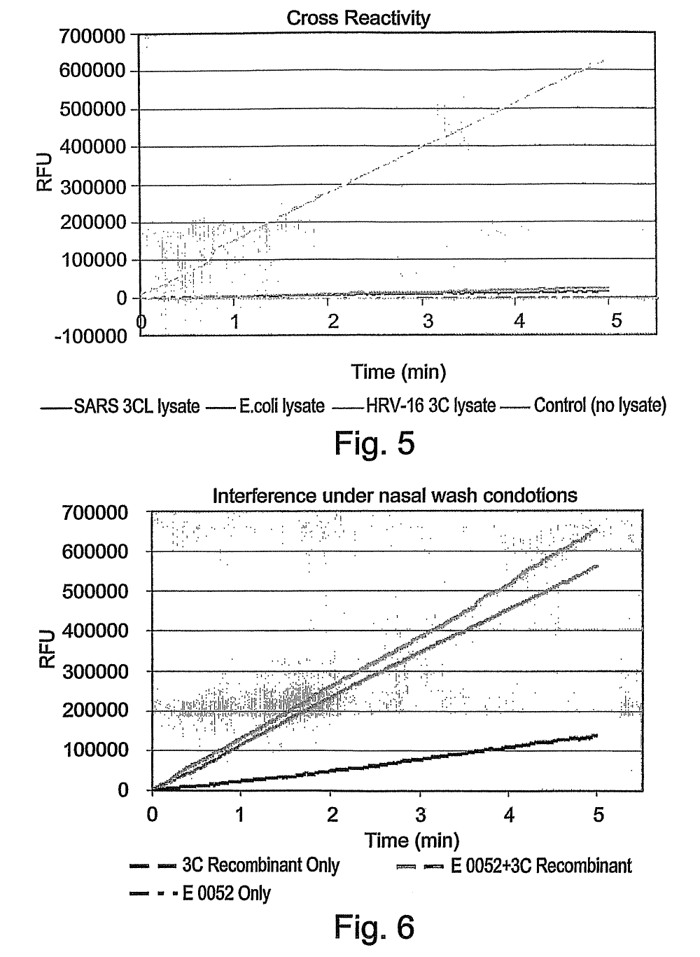

FIG. 5 shows the specificity of Pep1 for cleavage with HRV 3C protease in HRV 16 lysate, and the absence of any detectable cleavage reaction with SARS 3CL lysate or E. coli lysate.

Further tests for specificity were performed using H1 HeLa cell cultures to simulate an actual HRV infection in-situ, and test the efficacy of designed substrates in detection of 3C activity under such conditions. Cleared, sonicated lysates of the HRV infected HeLa cells were assayed using substrates Pep1, Ori 2 and Pep6. The results indicated that the HeLa cells have a high background of non-specific protease activity.

In an attempt to eliminate such non-specific effects in working with HeLa cells, the effects of a variety of known protease inhibitors (EDTA, EGTA, PMSF and Aprotinin) on the cleavage of designed substrate by HeLa lysate was assessed. Table 15 below illustrates the relative insensitivity of the protein cleavage reaction using Pep1 as substrate to the inhibitors PMSF and Aprotinin. EDTA/EGTA partially inhibited the 3C protease cleavage of Pep1.

TABLE 15

| | Control | Complete, | | | |
|---|---|---|---|---|---|
| | (H1 lysate | EDTA free | | | EDTA/ |
| | only, no | tablets | PMSF | Aprotinin | EGTA |
| | inhibitors) | (Sigma) | 2 mM | 8 ug/ml | 10 mM |
| Reaction rate | 119 | 110 | 119 | 115 | 65 |
| RFU/min | | | | | |
| % Inhibition | — | 7.5% | 0% | 3.3% | 45% |
| (relative | | | | | |
| to control) | | | | | |

Determining efficacy of 3C protease assay using optimal peptide substrate in clinical conditions: The clinical context for assay of HRV protease is characterized by the presence of mucosal secretions. In order to determine the efficacy of cleavage of designed substrates by 3C protease under actual clinical conditions, cleavage of Pep1 by recombinant 3C protease in the presence, and in the absence of added nasal wash samples was compared. FIG. 6 illustrates the absence of effect of nasal wash sample on the reaction kinetics, indicating the suitability of the reaction for clinical use.

Accurate detection of Rhinovirus viral infection by cleavage of optimal cleavage sequence substrate peptides in clinical nasal wash samples—In order to test the suitability of designed peptide substrates for use in the clinical setting, nasal wash samples obtained from subjects (using a mucus extractor kit—Mucosafe Extractor with filter—Maersk Medical A/S—Denmark) were evaluated for the presence of viruses, and then tested by the method of the invention for protease catalytic activity. A portion of each sample was assessed for the presence of RSV, influenza A and B viruses, parainfluenza viruses, and adenovirus by direct immunofluorescence assay (IFA), using commercial monoclonal antibodies (Chemicon International, Inc., Temecula, Calif.), and by tissue culture. The remainder was stored at −80° C. until analyzed for the presence of HRV.

Currently, a “gold standard” for HRV detection does not exist. Tissue culture is not a routine test due to virus susceptibility and there is no commercial immunoassay test available. Thus, RT-PCR analysis was selected, and the samples were analyzed at The Central Laboratory of Virology, Division of Infectious Diseases, Department of Internal Medicine, University Hospitals of Geneva, Geneva, Switzerland.

Clinical nasal washes tests: Assays were conducted using 24 clinical samples (nasal washes). Specific activity of 3C protease, using synthetic peptide substrate Pep1 was calculated relative to total protein concentration (by Bradford) (specific activity=RFU/min/mg protein).

Table 16 below shows the results of comparison of detection of HRV in the samples by RT-PCR and by protease activity (MND assay). 17 out of 24 samples determined protease-positive or protease-negative (specific activity greater or less than 0.5 RFU/min/mg protein, respectively) correlated with the RT-PCR results. Of the remaining 7 samples, 5 were negative in RT-PCR and positive according to the protease assay, and 2 were positive in RT-PCR and negative according to the protease assay. Statistical analysis of these preliminary results (Table 17), assuming the absolute accuracy of the RT-PCR, shows a 75% sensitivity compatibility and 70% specificity compatibility.

TABLE 16

| | Sample | | Additional | Specific activity | MND |
|---|---|---|---|---|---|
| | | RT-PCR | viruses | RFU/min/mg | results |
| | 2 uM | | | | |
| | substrate | | | Trash hold = 0.5 | |
| * | E0260 | N | | 0.49084 | N |
| * | E0265 | P | | 0.532999377 | P |
| * | E0269 | N | | 0 | N |
| * | E0281 | N | RSV PI AD | 0.296603037 | N |
| ** | E0282 | N | | 0.588661728 | P |
| * | E0283 | N | | 0 | N |
| ** | E0300 | N | CMV | 2.94504 | P |
| * | E0306 | N | | 0 | N |
| * | E0312 | N | | 0 | N |
| ** | E0313 | N | HPMV | 1.10124359 | P |
| * | E0314 | N | | 0.332420741 | N |
| * | E0317 | P | | 6.932318182 | P |
| | H1 HeLa | | | 0.191111111 | |
| | 4uM | | | | |
| | substrate | | | Trash hold = 5.3 | |
| * | E0320 | P | CMV | 19.30126825 | P |
| ** | E0335 | N | | 21.87407626 | P |
| * | E0336 | P | | 6.405326675 | P |
| ** | E0347 | N | PI | 16.08482439 | P |
| * | E0370 | N | | 0.658017493 | N |
| * | E0424 | P | | 9.198241407 | P |
| *** | E0474 | P | | 0.120745921 | N |
| *** | E0497 | P | | 0.334046092 | N |
| * | E0499 | N | | 0 | N |
| * | W0006 | N | | 5.290901444 | N |
| * | W0007 | N | AD | 3.075 | N |
| * | W0011 | P | | 9.290322581 | P |
| | H1 HeLa | | | 0.375 | |

P—Positive for HRV,
N—Negative for HRV,
PI—Para influenza virus,
AD—Adenovirus
* - correlation to RT-PCR,
** - RT-PCR negative MND positive,
*** - RT-PCR positive MND negative.

TABLE 17

RT-PCR

| | | Positive | Negative |
|---|---|---|---|
| MND | Positive | 6 | 5 |
| | Negative | 2 | 11 |

However, the accuracy of RT-PCR detection can be problematic, and protease detection using the method of the present invention has distinct advantages over detection by RT-PCR. RT-PCR only detects the RNA of the virus and therefore can also detect inactivated virus. Thus, it is likely that samples that were positive in RT-PCR and negative for protease activity are due to the fact that protease assay detects the virus only in its active form. Further, it will be appreciated that the samples tested were kept in storage at −80° C. for 3-4 years prior to use. Thus, samples that tested positive in RT-PCR and negative for protease may have had inactivated 3C protease due to the long period of storage. Yet further, protease detection may be more sensitive than RT-PCR, and this may be reflected in the samples testing negative in RT-PCR and positive for protease.

Thus, 3C protease assay using the optimal cleavage sequence substrate peptides successfully detected HRV 16 in clinical samples.

### Example 5

### Accurate Detection of Enterovirus Viral Infection by Cleavage of Optimal Cleavage Sequence Substrate Peptides in Clinical CSF Samples

Non-polio enterovirus (NPEV) infections are common throughout the late summer and early fall each year, and are second only to the rhinoviruses as the most common viral infectious agents in humans. Over 90% of aseptic meningitis cases are caused by NPEV, with classic symptoms being fever, severe headache, stiff neck, neck pain, nausea and vomiting, sensitivity to bright light, and possibly a rash.

Detection of NPEV in the CSF is currently performed by PCR and tissue cultures. NPEV PCR can provide results, within 5 to 24 hours of sample receipt with greater sensitivity and within a shorter time than viral culture. However, both tissue culture and RT-PCR are costly and require complicated equipment, unsuitable to rapid, on-site diagnostics. In order to test the efficacy of enterovirus detection by protease assay using optimal cleavage sequence substrate peptides, samples of human cerebro-spinal fluid (CSF) were assayed for NPEV by the method of the invention, and in the definitive tissue culture assay.

Clinical CSF tests: Table 18 illustrates the great sensitivity of the method of the present invention. Pep9 was selected as the optimal sequence substrate peptide for accurate and sensitive detection in the CSF. Pep9 was used for the following reasons; first, the proteases of NPEV and HRV belong to the same family showing that although non-optimal assay conditions are employed, results are still obtained. Second, since HRV does not reside in the CSF there is no risk of cross-reactivity. Fresh CSF was obtained by lumbar puncture.

TABLE 18

| CSF Sample | Cell/mm³ | MND's result | Tissue culture result |
|---|---|---|---|
| N1 | 450 | Positive | Positive |
| N2 | 4 | Negative | Negative |
| N3 | 2 | Negative | Negative |
| N4 | 1 | Negative | Negative |
| N5 | 148 | Negative | Negative |
| N6 | 194 | Negative | Negative |
| N7 | 4 | Negative | Negative |

Of the 7 fresh CSF samples, full correlation was found between the results of the tissue culture assay and the detection based on protease activity. Note that the positive correlation between the tissue culture results and the protease detection results was unaffected by the cell density (in the range of 450 cells/mm³-1 cell/mm³). Thus, accurate and rapid detection of enterovirus can be made using protease detection by cleavage of optimal cleavage sequence substrate peptides in CSF samples, under clinical conditions.

Taken together, these results show that optimal peptide substrates, designed and selected according to comparative enzyme kinetics, can be used to detect target enzymes of diagnostic interest, with great sensitivity and accuracy. Detection of such target enzymes of diagnostic interest can greatly improve rapid diagnosis of common diseases such as enterovirus and rhinovirus, and suspected epidemic and pandemic agents such as SARS and avian flu.

### Example 6

### Protease Detection Based on Separation

The present invention further provides a novel method for the simultaneous detection of one or more reaction agents (enzymes or specific chemicals) according to the specific substrate cleavage performed in the reaction, based on affinity separation is described. The detection of cleavage indicates the existence of a specific reaction which detection is required. In one embodiment one application of such method is for the detection of a specific enzyme that catalyses a specific reaction. In another embodiment, the enzyme is part of a biological system that its detection is required, in another embodiment the biological system is a virus or bacteria or another pathogen. The method of detection consists of a combination of two steps:

First—Separation between molecules that are processed in the reaction and molecules which are not processed. Second—Detection of processed molecules only.

The separation mechanism can utilize either the specific affinity binding of two moieties e.g. antibody-substrate, nucleic acid hybridization, or, on attachment to immobilized surface e.g. membranes, chips, beads etc. The detection step can be based either on affinity or any other way e.g. fluorimetric, colorimetric, enzymatic or both.

The substrate that undergoes the specific reaction to be detected is comprised of 3 parts: X, Y, Z and C (FIG. 7). The core molecule (segment Y e.g., kinetically optimal substrate, such as identified as described above) which has a specific cleavage site is connected in one end to a tagging molecule (segment X i.e., a detectable moiety), which purpose is to detect cleaved substrates. On the other end it is connected to a mechanism segment (segment Z, separating moiety) that separates between processed and unprocessed substrate. Upon cleavage of molecule Y the substrate cleavage products are formed: (1) Tagging Segment (TS, also referred to herein as X-Y′) that contains part X and a part of Y and (2) a Separating segment (SS. Also referred to herein as Y″-Z) that contain Z and a part of Y. The process that initiates the molecule cleavage and its detection is desired can be an enzymatic reaction, chemical reaction, denaturation or any other process.

The substrate described in FIG. 8 reacts with its designated cleaving moiety. Once cleavage had occurred, the Z segment (separating segment) is used to separate between the processed and unprocessed substrate. Therefore, only the TS of the processed substrate (that contains the detectable moiety) binds to the moiety with the high affinity to the detectable molecule. The affinity binding process is therefore detected only for cleaved substrates. Detection can be based on existing, known or novel methods. In this way it is possible to detect only molecules that were processed (FIG. 8).

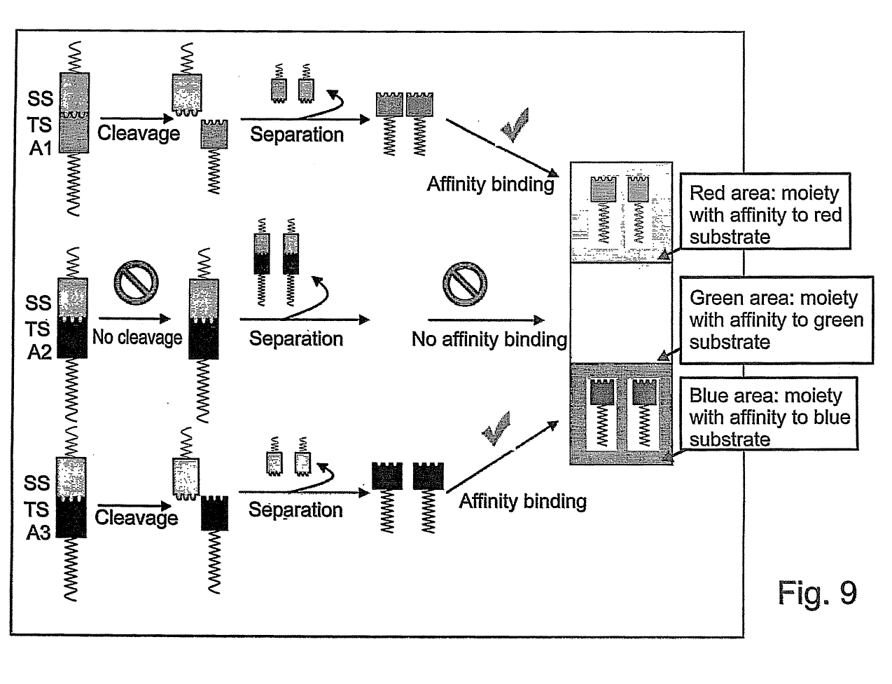

In another embodiment it is also possible to simultaneously detect the cleavage of a number of substrates using the above described method. This possibility occurs if the substrates are similar in their separating mechanism (Z) but different in their specific cleavage molecule (Y). In this case each substrate has a unique and different detectable moiety (X) that can be affiliated to its core molecule (Y). After cleavage and separating between processed and unprocessed substrates occurs, only the TS of the different (and processed) substrates are bound by affinity (in accordance to the above described method). Any molecule that contains Z (unprocessed substrates or SS of processed substrates) is retained by the separating mechanism. Further separation to the different TSs of the different substrates are obtained by designing a membrane, chip etc. on which each predetermined locus contain a moiety with affinity to each different TS, e.g. each locus can bind only to one kind of TS. The separated solution then comes in contact with this chip or membrane and affinity binding can occur. By knowing which predetermined loci are bound by affinity to the different TSs one can identify which substrates were processed. Since each substrate is specific to the enzyme that initiated the substrate cleavage it is possible to identify which of the enzymes has cleaved its corresponding substrate, deduce which protease, exists in the solution and consequently deduce which pathogen or agent corresponds to the corresponding enzyme (FIG. 9).

Reverse PH System (RPHS)—In this embodiment the C segment is a special molecule common to all substrates in the buffered solution. The A segments that correspond to the various substrates are dye molecular entities that their different colors are sensitive to different PHs. After cleavage the buffered solution is filtered through a column with affinity to segment C. Any molecule that contains segment C (unprocessed substrates or segment SS of processed substrates) will be retained at the column (by the affinity moiety corresponding to segment C). Only the TS segment of the processed substrates (that does not contain segment C) will be able to follow to a chamber that has a number of cells, each in different PH. Once the TS segment (that contains A) comes in contact with the cells (different PH) the cell changes color according to the properties of segment A. This indicates which substrates have been processed.

The Mechanism of Separation

The purpose of the separation mechanism is to separate between the TS and the SS in such a way that only the TS allows the binding to a moiety with affinity corresponding to the detectable moiety. In case of simultaneous detection of a number of substrates the separation mechanism has an additional role which is to remove the intact substrates in case that cleavage was not initiated for these substrates.

For the above described system any separation methods may be used. The following provided a brief description of such separation methods.

1. Immobilized Separation System (ISS)— in this system Z is a spacer linked to an immobilized surface via beads, nitrocellulose membrane, biotin-avidin or other affinity pair. After cleavage in a buffered solution any unprocessed substrate or the SS of the processed substrate is removed by separating the immobilized surface (by extraction, centrifugation, filtration etc.) from the buffered solution, leaving only the TS of the processed substrates. In this way it is also possible to monitor the kinetics of each substrate.

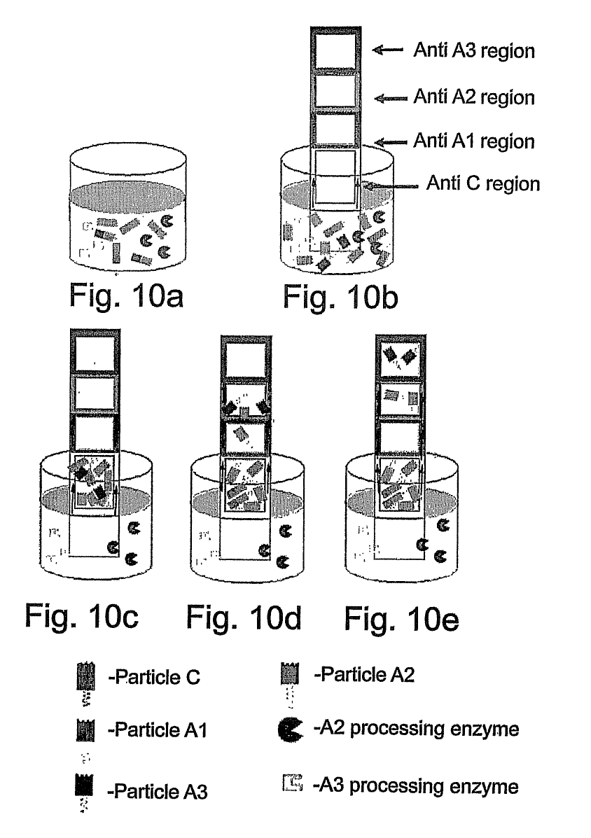

2. Dynamic Separation System (DSS)— in this system Z is a special molecule common or unique to all substrates in the buffered solution. After cleavage occurs, the buffered solution comes in contact with a specially designed membrane or chip. The membrane is vertical and at the bottom it has a moiety with affinity corresponding to Z. An adjacent part of the membrane includes different loci corresponding to X of the different substrates. The buffered solution is then pushed along the length of the membrane or chip by capillary or electro force. Any molecule that contains Z (unprocessed substrates or SS of processed substrates) will be retained at the bottom of the membrane (by the affinity moiety corresponding to Z). Only the TS of the processed substrates (do not contain Z) will be able to move up the membrane and bind by affinity to their predetermined loci (FIG. 10).

3. Affinity Filtration System (AFS) in this system the buffered solution is filtered trough a column with affinity to Z, thus any molecule that contains Z (unprocessed substrates or SS of processed substrates) will remain in the column. The flow trough will contain only the TS of the processed substrates.

Examples for Affinity Pairs

The affinity pairs for this system can be any affinity pair known in the art. Examples of affinity pairs include, but are not limited to, Biotin-Avidin, Antibody-Substrate; Receptor-Substrate; Sense-Anti-sense DNA/RNA strands, based on nucleic acid hybridization; PH dependent color molecule; Fluorescent—The detectable moiety can be based on FRET or other fluorescent detection method.

Examples for Detection Methods

Antibody/Receptor-substrate—Any existing or novel immunochemistry method either based on fluorescent or color is suitable.

Marker—The detectable moiety (X) can be or be attached to a molecule that produces color, fluorescent, FRET or any other measurable, visible or easily detectable molecule.

DNA Hybridization—Any existing or novel hybridization method such as based on fluorescent or color probe, is suitable.

Enzymatic reaction—The detectable moiety (X) can be attached to an enzyme that catalyzes color or fluorescent or any other measurable, visible or any other easily detectable reaction.

Protease Detection Based on Antibody Affinity

The presence of a number of viruses in a clinical sample based on their specific protease activity is detected. Many virus families utilize a specific protease activity as an important part of their life cycle. For example Rhinovirus (3C protease), Enterovirus (3C protease) and SARS (3CL protease) all utilize proteases with a unique cleavage sequence to their virus family.

In this example the substrate is a peptide cleavage sequence (corresponding to Y) connected in one end to biotin (corresponding to Z). The other end is connected to a molecule with affinity to a specific antibody (corresponding to X). Cleavage of this substrate will result in separation to SS and TS (as described above).

The reaction mixture contains different types of the above described substrate. All the substrates contain biotin moiety in one end. The peptides are different in the cleavage sequences corresponding to the specific protease and the unique antibody substrate that can be associated with a specific cleavage sequence (e.g. A1, A2 A3 . . . ). In this way each substrate can be associated with a different virus.

The reaction mixture is brought in contact with the clinical sample and cleavage can occur. After cleavage, the reaction mixture is analyzed on a membrane or chip, shaped as a strip and designed specifically for this purpose (FIG. 10). At the bottom it has a region with avidin. Above this region it has different loci with antibodies corresponding to the different antibody substrate of the different substrates [e.g. anti A1 (corresponding to anti X1), anti A2 (corresponding to anti X2), anti A3 (corresponding to anti X3).]. The buffered solution is then pushed along the length of the membrane or chip by capillary or electro force. Any molecule that contains biotin (unprocessed substrates or SS of processed substrates) will be retained at the bottom of the membrane (by the affinity to avidin). Only the TS of the processed substrates (do not contain biotin) will be able to move up the membrane and bind by affinity to their predetermined loci (e.g. A1-antiA1, A2-antiA2 etc, corresponding to the nomenclature described above).

Since each locus represents a different protease it is possible to determine from the presence of the processed substrates which proteases are present in the clinical sample. The presence of the protease confirms the presence of the corresponding specific virus.

Protease Detection Based on Nucleic Acid Hybridization

Sense and anti-Sense strands of DNA or RNA have a high affinity towards one another. The system utilizes this property. This system is designed with the same guidelines as described in the antibody example described above.

The difference is that the affinity binding is based on nucleic acid hybridization. In this example the substrate is a peptide cleavage sequence (corresponding to Y) connected in one end to a separating ssDNA strand (corresponding to Z). The other end is connected to a unique ssDNA strand (corresponding to X). Cleavage of this substrate results in separation to SS and TS (as described above).

The reaction mixture contains different types of the above described substrate. All the substrates have the same separating ssDNA strand. They are different in the cleavage sequences corresponding to the specific protease and the unique ssDNA that can be associated with a specific cleavage sequence (e.g. A1ssDNA, A2ssDNA, A3ssDNA . . . ). In this way each substrate can be affiliated to a different virus.

The reaction mixture comes in contact with the clinical sample and cleavage can occur. After cleavage, the reaction mixture is analyzed on a membrane or chip, shaped as a strip designed specifically for this purpose (FIG. 10). At the bottom in has a region with an anti-sense separating DNA strand. Above this region it has different loci with anti-sense DNA strand corresponding to the unique ssDNA of the different substrates (e.g. anti-sense A1ssDNA, anti-sense A2ssDNA, anti-sense A3ssDNA . . . ). The buffered solution is then pushed along the length of the membrane or chip by capillary or electro force. Any molecule that contains the separating ssDNA strand (unprocessed substrates or SS of processed substrates) is be retained at the bottom of the membrane (by the affinity to the anti-sense separating DNA). Only the TS of the processed substrates (do not contain separating ssDNA strand) is be able to move up the membrane and bind by affinity to their predetermined loci (e.g. A1ssDNA-anti-sense A1ssDNA, A2ssDNA-anti-sense A2ssDNA etc).

Since each locus represents a different protease it is possible to determine from the presence of the processed substrates which proteases are present in the clinical sample. The presence of the protease confirms the presence of the corresponding specific virus. Several viruses may be detected using single detectable moieties merely be the addressable location of the assay product on the solid phase.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

### Other References are Cited in the Document

1. http://virus.stanford.edu/adeno/adeno.html
2. http://www.tulane.edu/˜dmsander/WWW/335/Adenoviruses.html
3. THE JOURNAL OF BIOLOGICAL CHEMISTRY Vol. 270, No. 40, Issue of October 6, pp. 23250-23253, 1995
4. VIROLOGY 213, 494-502 (1995) The Adenovirus Protease Is Required for Virus Entry into Host Cells
5. J Biol. Chem. 1996 Dec. 20; 271(51):32511-4. Cleavage efficiency by adenovirus protease is site-dependent.
6. THE JOURNAL OF BIOLOGICAL CHEMISTRY Vol. 271, No. 51, Issue of December 20, pp. 32511-32514, 1996 Cleavage Efficiency by Adenovirus Protease Is Site-dependent*
7. http://www.socgenmicrobiol.org.uk/jgvdirect/19497/19497ft.htm
8. Rev. Med. Virol. 8: 213-222 (1998) Virus Assembly and Disassembly: the Adenovirus Cysteine Protease as a Trigger Factor
9. Ruzindana-Umunyana, A., Imbeault, L. & Weber, J. M. (2002). Substrate specificity of adenovirus protease. Virus Res 89, 41-52.
10. Bowden, D. S., and E. G. Westaway. 1984. Rubella virus structural and nonstructural proteins. J. Gen. Virol. 65:933-943.
11. Chen, J.-P., J. H. Strauss, E. G. Strauss, and T. K. Frey. 1996. Characterization of the rubella virus nonstructural protease domain and its cleavage site. J. Virol. 70:4707-4713.
12. 14. Gorbalenya, A. E., E. V. Koonin, and M. M.-C. Lai. 1991. Putative papainrelated thiol proteases of positive-strand RNA viruses. FEBS Lett. 288:201-205.
13. Gorbalenya, A. E., E. V. Koonin, and Y. I. Wolf. 1990. A new superfamily of putative NTP-binding domains encoded by genomes of small DNA and RNA viruses. FEBS Lett. 262:145-148.
14. Yao, J., D. Yang, P. Chong, D. Hwang, Y. Liang, and S. Gillam. 1998. Proteolytic processing of rubella virus nonstructural proteins. Virology 246: 74-82.
15. Liang Y, Yao J, Gillam S. Rubella virus nonstructural protein protease domains involved in trans- and cis-cleavage activities. J. Virol. 2000 June; 74(12):5412-23.
16. Liu X, Ropp S L, Jackson R J, Frey T K. The rubella virus nonstructural protease requires divalent cations for activity and functions in trans. J. Virol. 1998 May; 72(5):4463-6.
17. Bonilla, P. J., Hughes, S. A. & Weiss, S. R. (1997). Characterization of a second cleavage site and demonstration of activity in trans by the papain-like proteinase of the murine coronavirus mouse hepatitis virus strain A59. Journal of Virology 71, 900±909.
18. Snijder, E. J. & Meulenberg, J. J. M. (1998). The molecular biology of
19. arteriviruses. Journal of General Virology 79, 961±979.
20. Carrington, J. C., Freed, D. D. & Sanders, T. C. (1989). Autocatalytic processing of the potyvirus helper component proteinase in Escherichia coli and in vitro. Journal of Virology 63, 4459±4463.
21. Hardy, W. R., Hahn, Y. S., de Groot, R. J., Strauss, E. G. & Strauss, J. H. (1990). Synthesis and processing of the nonstructural polyproteins of several temperature-sensitive mutants of Sindbis virus. Virology 177,199±208.
22. Strauss, E. G., De Groot, R. J., Levinson, R. & Strauss, J. H. (1992). Identification of the active site residues in the nsP2 proteinase of Sindbis virus. Virology 191, 932±940.
23. Shirako, Y. & Strauss, J. H. (1990). Cleavage between nsP1 and nsP2 initiates the processing pathway of Sindbis virus nonstructural polyprotein P123. Virology 177, 54-64.
24. http://www.expasy.org/uniprot/Q8JJX1.
25. Hardy, W. R. & Strauss, S. H. (1989). Processing the nonstructural polyproteins of Sindbis virus: nonstructural proteinase is in the C terminal half of nsP2 and functions both in cis and in trans. Journal of Virology 63, 4653±4664.
26. De Groot, R. J., Hardy, W. R., Shirako, Y. & Strauss, J. H. (1990). Cleavage-site preferences of Sindbis virus polyproteins containing the non-structural proteinase. Evidence for temporal regulation of polyprotein processing in vivo. EMBO Journal 9, 2631±2638.
27. Ten Dam E, Flint M, Ryan M D. Virus-encoded proteinases of the Togaviridae. J Gen Virol. 1999 August; 80 (Pt 8):1879-88.
28. http://www.freshpatents.com/Optimized-recognition-site-of-the-alphavirus-non-stuctural-protease-for-tag-removal-and-specific-processing-of-recombinant-proteins-dt20051222ptan20050282254.php?type=description
29. Halin, C. S., Strauss, E. G. & Strauss, J. H. Sequence analysis of three Sindbis virus mutants temperature-sensitive in the capsid protein autoprotease. Proc. Natl Acad. Sci. USA 82, 4648-4652 (1985).
30. Hahn, C. S. & Strauss, J. H. Site-directed mutagenesis of the proposed catalytic amino acids of the Sindbis virus capsid protein autoprotease. J. Virol. 64, 3069-3073 (1990).
31. Melancon, P. & Garoff, H. (1987). Processing of the Semliki Forest virus structural polyprotein: role of the capsid protease. Journal of Virology 61, 1301±1309.
32. Strauss, J. H. & Strauss, E. G. The alphaviruses: gene expression, replication, and evolution. Microbiol. Rev. 58, 491-562 (1994).
33. Takkinen, K., Peranen, J. & Kaariainen, L. (1991). Proteolytic processing of Semliki Forest virus-specific non-structural polyprotein. Journal of General Virology 72, 1627±1633.
34. Jacks, T., M. D. Power, F. R. Masiarz, P. A. Luciw, P. J. Barr, H. E. Varmus. 1998. Characterization of ribosomal frameshifting in HIV-1 gag-pol expression. Nature 331:280-283.
35. Wondrak, E. M., Louis, J. M., De Rooquigny, H., Chermann, J.-C., And Roques, B. P. (1993). The Gag Precursor Contains A Specific HIV-1 Protease Cleavage site between the NO (pT) and P1 proteins. FEBS Lett. 333, 21-24.
36. Billich, S., M. T. Knoop, J. Hansen, P. Strop, J. Sedlacek, R. Mertz, and K. Moelling. 1988. Synthetic peptides as substrates and inhibitors of human immunodeficiency virus-1 protease. J. Biol. Chem. 263:17905-17908.
37. Berkhout B. HIV-1 evolution under pressure of protease inhibitors: climbing the stairs of viral fitness. J Biomed Sci 1999, 6: 298-305.
38. Cote H C, Brumme Z L, Harrigan P R. Human immunodeficiency virus type 1 protease cleavage site mutations associated with protease inhibitor cross-resistance selected by indinavir, ritonavir, and/or saquinavir.
39. J. Virol. 2001 January; 75(2):589-94.
40. Kotler, M., Katz, R. A, Danho, W., Leis, J., and Skalka, A. M. (1988) Proc.
41. Natl. Acad. Sci. U.S.A. 86, 4185-4189
42, Rose J R, Salto R, Craik C S. Regulation of autoproteolysis of the HIV-1 and HIV-2 proteases with engineered amino acid substitutions.
43. J Biol. Chem. 1993 Jun. 5; 268(16):11939-45.
44. To\ddot{}zse´r, J., A. Gustchina, I. T. Weber, I. Blaha, E. M. Wondrak, and S. Oroszlan. 1991. Studies on the role of the S4 substrate binding site of HIV proteinases. FEBS Lett. 279:356-360.
45. To\ddot{}zse´r, J., I. T. Weber, A. Gustchina, I. Blaha, T. D. Copeland, J. M. Louis,
46. and S. Oroszlan. 1992. Kinetic and modeling studies of S3-S3_subsites of
47. HIV proteinases. Biochemistry 31:4793-4800.
48. Bagossi P, Sperka T, Feher A, Kadas J, Zahuczky G, Miklossy G, Boross P, Tozser J. Amino acid preferences for a critical substrate binding subsite of retroviral proteases in type 1 cleavage sites. J Virol. 2005 April; 79(7):4213-8.

49. Fehe´r, A., I. T. Weber, P. Bagossi, P. Boross, B. Mahalingam, J. M. Louis, T. D. Copeland, I. Y. Torshin, R. W. Harrison, and J. To\ddot{}zse´r. 2002. Effect of sequence polymorphism and drug resistance on two HIV-1 Gag processing sites. Eur. J. Biochem. 269:4114-4120.
50. Matayoshi, E. D., et al., Novel fluorogenic substrates for assaying retroviral proteases by resonance energy transfer. Science, 247, 954-958 (1990).
51. http://www.sigmaaldrich.com/sigma/datasheet/h6660dat.pdf.
52. http://www.sigmaaldrich.com/sigma/datasheet/h6660dat.pdf
53. To\ddot{}zse´r, J., P. Bagossi, I. T. Weber, J. M. Louis, T. D. Copeland, and S. Oroszlan. 1997. Studies on the symmetry and sequence context dependence of the HIV-1 proteinase specificity. J. Biol. Chem. 272:16807-16814.
54. http://bioafrica.mrc.ac.za/proteomics/HIVcleavagesites.html#ma-ca
55. Poiesz, B. J., Ruscetti, F. W., Gazdar, A. F., Bunn, P. A., Minna, J. D. & Gallo, R. C. (1980) Detection and isolation of type C retrovirus particles from fresh and cultured lymphocytes of a subject with cutaneous T-cell lymphoma. Proc. Natl Acad. Sci. USA 77, 7415±7419.
56. Sheremata, W. A., Benedict, D., Squilacote, D.C., Sazant, A. & DeFreitas, E. (1993) High-dose zidovudine induction in HTLV-I associated myelopathy: safety and possible efficacy. Neurology 43, 2125-2129.
57. Oroszlan, S. & Luftig, R. B. (1990) Retroviral proteinases. Curr. Top. Microbiol. Immunol. 157, 153±185.
58. Hatanaka M, Nam S H. Identification of HTLV-I gag protease and its sequential processing of the gag gene product. J Cell Biochem. 1989 May; 40(1):15-30.
59. Tozser J, Zahuczky G, Bagossi P, Louis J M, Copeland T D, Oroszlan S, Harrison R W, Weber I T. Comparison of the substrate specificity of the human T-cell leukemia virus and human immunodeficiency virus proteinases. Eur J Biochem. 2000 October; 267(20):6287-95.
60. Louis J M, Oroszlan S, Tozser J. Stabilization from autoproteolysis and kinetic characterization of the human T-cell leukemia virus type 1 proteinase. J Biol Chem. 1999 Mar. 5; 274(10):6660-6.
61. V. Thiel, J. Herold, B. Schelle, S. G. Siddell, J. Virol. 75, 6676 (2001).
62. J. Ziebuhr, J. Herold, S. G. Siddell, J. Virol. 69, 4331 (1995).
63. Gorbalenya, A. E., Koonin, E. V. & Lai, M. M. (1991). Putative papain-related thiol proteases of positive-strand RNA viruses. Identification of rubi- and aphthovirus proteases and delineation of a novel conserved domain associated with proteases of rubi-, alpha- and coronaviruses. FEBS Lett 288, 201-205.
64. J. Ziebuhr, E. J. Snijder, A. E. Gorbalenya, J. Gen. Virol. 81, 853 (2000).
65. Ziebuhr, J., Snijder, E. J. & Gorbalenya, A. E. (2000). Virus-encoded proteinases and proteolytic processing in the Nidovirales. J Gen Virol 81, 853-79.
66. Rota, P. A., Oberste, M. S., Monroe, S, S., Nix, W. A., Campagnoli, R., Icenogle, J. P., Penaranda, S., Bankamp, B., Maher, K., Chen, M. H., Tong, S., Tamin, A., Lowe, L., Frace, M., DeRisi, J. L., Chen, Q., Wang, D., Erdman, D. D., Peret, T. C., Burns, C., Ksiazek, T. G., Rollin, P. E., Sanchez, A., Liffick, S., Holloway, B., Limor, J., McCaustland, K., Olsen-Rasmussen, M., Fouchier, R., Gunther, S., Osterhaus, A. D., Drosten, C., Pallansch, M. A., Anderson, L. J. & Bellini, W. J. (2003). Characterization of a novel coronavirus associated with severe acute respiratory syndrome. Science 300, 1394-1399.
67. Snijder, E. J., Bredenbeek, P. J., Dobbe, J. C., Thiel, V., Ziebuhr, J., Poon, L. L. M., Guan, Y., Rozanov, M., Spaan, W. J. M. & Gorbalenya, A. E. (2003). Unique and conserved features of genome and proteome of SARS coronavirus, an early split-off from the coronavirus group 2 lineage. J Mol Biol, submitted.
68. M. Marra et al., (2003) The Genome sequence of the SARS-associated coronavirus. Science 300, 1399-1404.
69. Ziebuhr, J., Thiel, V. & Gorbalenya, A. E. (2001). The autocatalytic release of a putative RNA virus transcription factor from its polyprotein precursor involves two paralogous papain-like proteases that cleave the same peptide bond. J Biol Chem 276, 33220-33232.
70. Hegyi A, Ziebuhr J. Conservation of substrate specificities among coronavirus main proteases. J Gen Virol. 2002 March; 83(Pt 3):595-9.
71. Fan K, Wei P, Feng Q, Chen S, Huang C, Ma L, Lai B, Pei J, Liu Y, Chen J, Lai L. Biosynthesis, purification, and substrate specificity of severe acute respiratory syndrome coronavirus 3C-like proteinase. J Biol Chem. 2004 Jan. 16; 279(3):1637-42. Epub 2003 Oct. 15.
72. Huang C, Wei P, Fan K, Liu Y, Lai L. 3C-like proteinase from SARS coronavirus catalyzes substrate hydrolysis by a general base mechanism. Biochemistry. 2004 Apr. 20; 43(15):4568-74.
73. Saskia L. Smits, Eric J. Snijder, and Raoul J. de Groot Characterization of a Torovirus Main Proteinase J Virol. 2006 April; 80(8): 4157-4167.
74. Ziebuhr, J., S. Bayer, J. A. Cowley, and A. E. Gorbalenya. 2003. The 3C-like proteinase of an invertebrate nidovirus links coronavirus and potyvirus homologs. J. Virol. 77:1415-1426.
75. Anand, K., G. J. Palm, J. R. Mesters, S. G. Siddell, J. Ziebuhr, and R. Hilgenfeld. 2002. Structure of coronavirus main proteinase reveals combination of a chymotrypsin fold with an extra alpha-helical domain. EMBO J. 21:3213-3224.
76. Barrette-Ng, I. H., K. K. Ng, B. L. Mark, D. Van Aken, M. M. Cherney, C. Garen, Y. Kolodenko, A. E. Gorbalenya, E. J. Snijder, and M. N. James. 2002. Structure of arterivirus nsp4. The smallest chymotrypsin-like proteinase with an alpha/beta C-terminal extension and alternate conformations of the oxyanion hole. J. Biol. Chem. 277:39960-39966.
77. Anand, K., J. Ziebuhr, P. Wadhwani, J. R. Mesters, and R. Hilgenfeld. 2003. Coronavirus main proteinase (3CLpro) structure: basis for design of anti-SARS drugs. Science 13:13,
78. Den Boon, J. A., Snijder, E. J., Chirnside, E. D., de Vries, A. A. F., Horzinek, M. C., and Spaan, W. J. M. (1991) J. Virol. 65, 2910-2920
79. Snijder, E. J., Wassenaar, A. L. M., and Spaan, W. J. M. (1992) J. Virol. 66, 7040-7048
80. Van Dinten L C, Rensen S, Gorbalenya A E, Snijder E J. Proteolytic processing of the open reading frame 1b-encoded part of arterivirus
81. replicase is mediated by nsp4 serine protease and Is essential for virus replication. J. Virol. 1999 March; 73(3):2027-37.
82. Snijder E J, Wassenaar A L, van Dinten L C, Spaan W J, Gorbalenya A E. The arterivirus nsp4 protease is the prototype of a novel group of chymotrypsin-like enzymes, the 3C-like serine proteases. J Biol. Chem. 1996 Mar. 1; 271(9):4864-71.
83. proteolytic activity of human cytomegalovirus u180 protease cleavage site mutants Thomas R. Jones,* Lei Sun, Geraldine A. Bebernitz, Viera P. Muzithras, Hong-Jin Kim, Stuart H. Johnston, and Ellen Z. Baum
84. Peptide Substrate Cleavage Specificity of the Human Cytomegalovirus Protease
85. Liu, F., and B. Roizman. 1991. The herpes simplex virus 1 gene encoding a protease also contains within its coding domain the gene encoding the more abundant substrate. J. Virol. 65:5149-5156.
86. Preston, V. G., F. J. Rixon, I. M. McDougall, M. McGregor, and M. F. Al Kobaisi. 1992. Processing of the herpes simplex virus assembly protein ICP35 near its carboxy terminal end requires the product of the whole of the UL26 reading frame. Virology 186:87-98.
87. Preston, V. G., J. A. Coates, and F. J. Rixon. 1983. Identification and characterization of a herpes simplex virus gene product required for encapsidation of virus DNA. J. Virol. 45:1056-1064.
88. Welch A R, McNally L M, Hall M R, Gibson W. Herpesvirus proteinase: site-directed mutagenesis used to study maturational, release, and inactivation cleavage sites of precursor and to identify a possible catalytic site serine and histidine. J Virol. 1993 December; 67(12):7360-72.
89. Liu, F., and B. Roizman. 1992. Differentiation of multiple domains in the herpes simplex virus 1 protease encoded by the UL26 gene. Proc. Natl. Acad. Sci. USA 89:2076-2080.
90. Baum, E. Z., G. A. Bebemitz, J. D. Hulmes, V. P. Muzithras, T. R. Jones, and Y. Gluzman. 1993. Expression and analysis of the human cytomegalovirus UL80-encoded protease: identification of auto proteolytic sites. J. Virol. 67:497-506.
91. Qiu X Y, Culp J S, Dilella A G, Hellmig B, Hoog S S, Janson C A, Smith W W, Abdelmeguid S S. Unique fold and active site in cytomegalovirus protease. Nature 1996; 383:275-279.
92. Hoog S S, Smith W W, Qiu X, Janson C A, Hellmig B, McQueney M S, O'Donnell K, O'Shannessy D, Dilella A G, Debouck C, Abdelmeguid S S. Active site cavity of herpesvirus proteases revealed by the crystal structure of herpes simplex virus protease/inhibitor complex. Biochemistry 1997; 36: 14023-14029.
93. Qiu X, Janson C A, Culp J S, Richardson S B, Debouck C, Smith W W, Abdelmeguid S S. Crystal structure of varicella-zoster virus protease. Proc Natl Acad Sci USA 1997; 94:2874-2879.
94. Reiling K K, Pray T R, Craik C S, Stroud R M. Functional consequences of the Kaposi's sarcoma-associated herpesvirus protease structure: Regulation of activity and dinerization by conserved structural elements. Biochemistry 2000; 39: 12796-12803.
95, Buisson M, Hernandez J-F, Lascoux D, Schoehn G, Forest E, Arlaud G, Seigneurin J-M, Ruigrok R W H, Burmeister W P. The crystal structure of the Epstein-Barr virus protease shows rearrangement of the processed C terminus. J Mol Bio 2002; 324:89-103.
96. Herpes proteases reviews. (a) Holwerda BC. Herpesvirus proteases: Targets for novel antiviral drugs. Antiviral Res 1997; 35:1-21. (b) Qiu X Y, Abdelmeguid S S. Human Herpes Proteases. In: Dunn B M, editor. Proteases of Infectious Agents. Chapter 3. San Diego: Academic Press; 1999. pp. 93-115. (c) Waxman L, Darke P L. The herpes proteases as targets for antiviral chemotherapy. Antiviral Chem Chemother 2000; 11:1-22
97. Gibson W, Welch A R, Hall M R T. Assemblin, a herpesvirus serine maturational proteinase and a new molecular target for antivirals. Perspect Drug Discov Design 1994; 2:413-426.
98. Haley T M, Angier S J, Borthwick A D, Montogomery D S, Purivs I J, Smart D H, Bessant C, Van Wely C, Hart G J. Investigation of the covalent modification of the catalytic triad of human cytomegalovirus protease by pseudo-reversible â-lactam inhibitors and a peptide chloromethyl ketone. J Mass Spectrom 1998; 33:1246-1255.
99. Borthwick A D, Weingarten G G, Haley T M, Tomaszewski M, Wang W, Hu Z, Bedard J, Jin H, Yeun L, Mansour T S. Design and synthesis of monocyclic b-lactams as mechanism-based inhibitors of human cytomegalovirus protease. Bioorg Med Chem Lett 1998; 8:365-370.
100. W. Gibson, A. R. Welch and M. R. T. Hall, Perspect. Drug Discov. Desi. 2,413 (1994).
101. H. S. Shieh, R. G. Kurumbail, A. M. Stevens, R. A. Stegeman, E. J. Sturman, J. Y. Pak, A. J. Wittwer, M. O. Palmier, R. C. Wiegand, B. C. Holwerda and W. C. Stallings, Nature (London) 383, 279 (1996).
102. http://www.expasy.org/cgi-bin/ft_viewer.pl?P10210
103. http://www.ncbi.nlm.nih.gov/entrez/viewer.fcgi?db=protein&val=139237
104. http://www.ncbi.nlm.nih.gov/entrez/viewer.fcgi?db=protein&val=139231.
105. http://72.14.207.104/search?q=cache:tZ7siCbEuq4J:www.labtestsonline.org/understanding/analytes/cmv/test.html+%22CMV++strain %22+pregnancy&hl=iw&ct=clnk&cd=3.
106. Barrett, A. D., 2001. Current status of flavivirus vaccines. Ann. N.Y. Acad. Sci. 951, 262-271.
107. Chambers, T. J., C. S. Hahn, R. Galler, and C. M. Rice. 1990. Flavivirus genome organization, expression and replication. Annu. Rev. Microbiol. 44:649-688.
108. Biedrzyclca, A., M. R. Cauchi, A. Bartholomeusz, J. J. Gorman, and P. J. Wright. 1987. Characterization of protease cleavage sites involved in the formation of envelope glycoprotein and three nonstructural proteins of dengue virus type 2, New Guinea C strain. J. Gen. Virol. 68:1317-1326.
109. Lin, C., S. M. Amberg, T. J. Chambers, and C. M. Rice. 1993. Cleavage at a novel site in the NS4A region by the yellow fever virus NS2B-3 proteinase is a prerequisite for processing at the downstream 4A/4B signalase site. J. Virol. 67:2327-2335.
110. Gorbalenya, A. E., A. P. Donchenko, V. M. Blinov, and E. V. Koonin. 1989. Cysteine proteases of positive strand RNA viruses and chymotrypsin-like serine proteases: a distinct protein superfamily with a common structural fold. FEBS Lett. 243:103-114.
111. Arias, C. F., Preugschat, F., Strauss, J. H., 1993. Dengue 2 virus NS2B and NS3 form a stable complex that can cleave NS3 within the helicase domain. Virology 193 (2), 888-899.
112. Chambers, T. J., R. C. Weir, A. Grakoui, D. W. McCourt, J. F. Bazan, R. J. Fletterick, and C. M. Rice. 1990. Evidence that the N-terminal domain of nonstructural protein NS3 from yellow fever virus is a serine protease responsible for site-specific cleavages in the viral polyprotein. Proc. Natl. Acad. Sci. USA 87:8898-8902.
113. Stocks, C. E., and M. Lobigs. 1998. Signal peptidase cleavage at the flavivirus C-prM junction: dependence on the viral NS2B-3 protease for efficient processing requires determinants in C, the signal peptide, and prM. J. Virol. 72:2141-2149.
114. Kummerer, B. M., Rice, C. M., 2002. Mutations in the yellow fever virus nonstructural protein NS2A selectively block production of infectious particles. J. Virol. 76 (10), 4773-4784.
115. Lin, C., Amberg, S. M., Chambers, T. J., Rice, C. M., 1993. Cleavage at a novel site in the NS4A region by the yellow fever virus NS2B-3 proteinase is a prerequisite for processing at the downstream 4A/4B signalase site. J. Virol. 67 (4), 2327-2335.
116. Ryan, M. D., Monaghan, S., Flint, M., 1998. Virus-encoded proteinases of the Flaviviridae. J. Gen. Virol. 79 (Pt 5), 947-959.
117. Rice, C. M., E. M. Lenches, S. R. Eddy, S. J. Shin, R. L. Sheets, and J. H. Strauss. 1985. Nucleotide sequence of yellow fever virus: implication for flavivirus gene expression and evolution. Science 229:726-733.
118. Yusof, R., Clum, S., Wetzel, M., Murthy, H. M., and Padmanabhan, R. (2000) J. Biol. Chem. 275, 9963-9969
119. Heinz, F. X., K. Stiasny, G. Pu\ddot{}schner-Auer, H. Holzmann, S. L. Allison, C. W. Mandl, and C. Kunz. 1994. Structural changes and functional control of the tick-borne encephalitis virus glycoprotein E by the heterodimeric association with protein prM. Virology 198:109-117.
120. Lobigs M. Flavivirus premembrane protein cleavage and spike heterodimer secretion require the function of the viral proteinase NS3.
121. Proc Natl Acad Sci USA. 1993 Jul. 1; 90(13):6218-22.
122. Yamshchikov V F, Compans R W. Formation of the flavivirus envelope: role of the viral NS2B-NS3 protease. J Virol. 1995 April; 69(4):1995-2003.
123. Lin, C., S. M. Amberg, T. J. Chambers, and C. M. Rice. 1993. Cleavage at a novel site in the NS4A region by the yellow fever virus NS2B-3 proteinase is a prerequisite for processing at the downstream 4A/4B signalase site. J. Virol. 67:2327-2335.
124. Nestorowicz, A., T. J. Chambers, and C. M. Rice. 1994. Mutagenesis of the yellow fever virus NS2A/2B cleavage site: effects on proteolytic processing, viral replication and evidence for alternative processing of the NS2A protein. Virology 199:114-123.
125. Yusof R, Clum S, Wetzel M, Krishna Murthy H M and Padmanabhan R. Purified NS2B/NS3 serine protease of dengue virus type 2 exhibits cofactor NS2B dependence for cleavage of substrates with dibasic amino acids. J Biol Chem, 2000, 275: 9963-9969.
126. Khumthong R, Niyomrattanakit P, Chanprapaph S, Angsuthanasombat C, Panyin S and Katzenmeier G. Steady-state cleavage kinetics for dengue virus type 2 NS2B-NS3(pro) serine protease with synthetic peptides. Prot Pept Lett, 2003, 10: 19-26.
127. Li, J., Lim, S. P., Beer, D., Patel, V., Wen, D., Tumanut, C., Tully, D. C., Williams, J. A., Jiricek J., Priestle, J. P., Harris, J. L., Vasudevan, S. G., 2005. Functional profiling of recombinant NS3 proteases from all four serotypes of dengue virus using tetrapeptide and octapeptide substrate libraries. J. Biol. Chem. 280 (31), 28766-28774
128. Yusof R, Clum S, Wetzel M, Krishna Murthy H M and Padmanabhan R. Purified NS2B/NS3 serine protease of dengue virus type 2 exhibits cofactor NS2B dependence for cleavage of substrates with dibasic amino acids. J Biol Chem, 2000, 275: 9963-9969.
129. Khumthong R, Niyomrattanakit P, Chanprapaph S, Angsuthanasombat C, Panyim S and Katzenmeier G. Steady-state cleavage kinetics for dengue virus type 2 NS2B-NS3(pro) serine protease with synthetic peptides. Prot Pept Lett, 2003, 10: 19-26.
130. Khumthong R, Angsuthanasombat C, Panyim S, Katzenmeier G. In vitro determination of dengue virus type 2 NS2B-NS3 protease activity with fluorescent peptide substrates. J Biochem Mol. Biol. 2002 Mar. 31; 35(2): 206-12.
131. Yusof, R., Clum, S., Wetzel, M., Krishn a Murthy, H. M. and Padmanabhan, R. (2000) Purified NS2B/NS3 serine protease of dengue virus type 2 exhibits cofactor NS2B dependence for cleavage of substrates with dibasic amino acids in vitro. J. Biol. Chem. 275, 9963-9969.
132. Chambers T J, Grakoui A, Rice C M. Processing of the yellow fever virus nonstructural polyprotein: a catalytically active NS3 proteinase domain and NS2B are required for cleavages at dibasic sites.
133. J. Virol. 1991 November; 65(11):6042-50.
134. Grakoui, A., D. W. McCourt, C. Wychowski, S. M. Feinstone, and C. M. Rice. 1993. A second hepatitis C virus-encoded proteinase. Proc. Natl. Acad. Sci. USA 90:10583-10587.
135. Reed K E, Grakoui A, Rice C M. Hepatitis C virus-encoded NS2-3 protease: cleavage-site mutagenesis and requirements for bimolecular cleavage.
136. J Virol. 1995 July; 69(7):4127-36
137. Bartenschlager, R., L. Ahlborn-Laake, J. Mous, and H. Jacobsen. 1994. Kinetic and structural analysis of hepatitis C virus polyprotein processing. J. Virol. 68:5045-5055.
138. Failla, C., L. Tomei, and R. De Francesco. 1994. Both NS3 and NS4A are required for proteolytic processing of hepatitis C virus nonstructural protein. J. Virol. 68:3753-3760.
139. Grakoui, A., C. Wychowski, C. Lin, S. M. Feinstone, and C. M. Rice. 1993. Expression and identification of hepatitis C virus polyprotein products. J. Virol. 67:1385-1395.
140. Pacini L, Vitelli A, Filocamo G, Bartholomew L, Brunetti M, Tramontano A, Steinkuhler C, Migliaccio G. In vivo selection of protease cleavage sites by using chimeric Sindbis virus libraries. J Virol. 2000 November; 74(22):10563-70.
141. A A Kolykhalov, E V Agapov and C M Rice Specificity of the hepatitis C virus NS3 serine protease: effects of substitutions at the 3/4A, 4A/4B, 4B/5A, and 5A/5B cleavage sites on polyprotein processing. J. Virol. 1994 Nov. 68(11), 7525-7533.
142. Urbani A, Bianchi E, Narjes F, Tramontano A, De Francesco R, Steinkuhler C, Pessi A. Substrate specificity of the hepatitis C virus serine protease NS3. J Biol. Chem. 1997 Apr. 4; 272(14):9204-9.
143. Collett, M. S., M. A. Wiskerchen, E. Welniak, and S. K. Belzer. 1991. Bovine viral diarrhea virus genomic organization. Arch. Virol. 3 (Suppl.):19-27.
144. Tautz N, Elbers K, Stoll D, Meyers G, Thiel H J. Serine protease of pestiviruses: determination of cleavage sites. J Virol. 1997 July; 71(7):5415-22.
145. http://content.karger.com/ProdukteDB/produkte.asp?Aktion=ShowPDF&ProduktNr=224031&Ausgabe=224837&ArtikelNr=24967&filename24967.pdf
146. Pinto R M, Guix S, Gonzalez-Dankaart J F, Caballero S, Sanchez G, Guo K J, Ribes E, Bosch A. Hepatitis A virus polyprotein processing by Escherichia coli proteases. J Gen Virol. 2002 February; 83 (Pt 2):359-68.
147. Harmon S A, Emerson S U, Huang Y K, Summers D E, Ehrenfeld E. Hepatitis A viruses with deletions in the 2A gene are infectious in cultured cells and marmosets. J Virol. 1995 September; 69(9):5576-81.
148. Probst, C., M. Jecht, and V. Gauss-Mu\ddot{}ller. 1998. Processing of proteinase precursors and their effect on hepatitis A virus particle formation. J. Virol. 72:8013-8020.
149. Schultheiss, T., Y. Y. Kusov, and V. Gauss-Mu\ddot{}ller. 1994. Proteinase 3C of hepatitis A virus (HAV) cleaves the HAV polyprotein P2-P3 at all sites including VP1/2A and 2A/2B. Virology 198:275-281.
150. Schultheiss T, Sommergruber W, Kusov Y, Gauss-Muller V. Cleavage specificity of purified recombinant hepatitis A virus 3C proteinase on natural substrates. J Virol. 1995 March; 69(3): 1727-33.
151. Kusov Y, Gauss-Muller V. Improving proteolytic cleavage at the 3A/3B site of the hepatitis A virus polyprotein impairs processing and particle formation, and the impairment can be complemented in trans by 3AB and 3ABC. J Virol. 1999 December; 73(12):9867-78.
152. A continuous colorimetric assay for rhinovirus-14 3C protease using peptide p-nitroanilides as substrates. Wang, Q. May; Johnson, Robert B.; Cox, Gregory A.; Villarreal, Elcira C.; Loncharich, Richard J. Infectious Diseases Research, Lilly Research Laboratories, Eli Lilly and Company, Indianapolis, Ind., USA. Anal. Biochem. (1997), 252(2), 238-245.
153. Development of a continuous fluorescence assay for rhinovirus 14 3C protease using synthetic peptides. Wang, Q. M.; Johnson, R. B.; Cohen, J. D.; Voy, G. T.; Richardson, J. M.; Jungheim, L. N. Infectious Diseases Res., Lilly Res. Labs., Eli Lilly and Co., Indianapolis, Ind., USA. Antiviral Chem. Chemother. (1997), 8(4), 303-310.
154. Rhinovirus 3C protease catalyzes efficient cleavage of a fluorescein-labeled peptide affording a rapid and robust assay. Hopkins, Jerry L.; Betageri, Rajashekhar; Cohen, Kenneth A.; Emmanuel, Michel J.; Joseph, Cathleen R.; Bax, Patricia M.; Pallai, Peter V.; Skoog, Mark T. Dep. Anal. Chem., Boehringer Ingelheim Pharm. Inc., Ridgefield, Conn., USA. J. Biochem. Biophys. Methods (1991), 23(2), 107-13.
155. Substrate requirements of human rhinovirus 3C protease for peptide cleavage in vitro. Cordingley, Michael G.; Callahan, Pia L.; Sardana, Vinod V.; Garsky, Victor M.; Colonno, Richard J. Dep. Virus Cell Biol., Merck Sharp and Dohme Res. Lab., West Point, Pa., USA. J. Biol. Chem. (1990), 265(16), 9062-5.
156. Cleavage of small peptides in vitro by human rhinovirus 14 3C protease expressed in Escherichia coli. Cordingley, Michael G.; Register, R. Bruce; Callahan, Pia L.; Garsky, Victor M.; Colonno, Richard J. Dep. Virus Cell Biol., Merck Sharp and Dohme Res. Lab., West Point, Pa., USA. J. Virol. (1989), 63(12), 5037-45.
157. The expression and purification of human rhinovirus protease 3C. Knott, Jacqueline A.; Orr, David C.; Montgomery, Douglas S.; Sullivan, Carole A.; Weston, Anthony. Genet. Dep., Glaxo Group Res., Greenford/Middlesex, UK. Eur. J. Biochem. (1989), 182(3), 547-55.
158. Human rhinovirus 3C protease: cloning and expression of an active form in Escherichia coli. Libby, Randell T.; Cosman, David; Cooney, Marion K.; Merriam, Janet E.; March, Carl J.; Hopp, Thomas P. Dep. Mol. Biol., Immunex Corp., Seattle, Wash., USA. Biochemistry (1988), 27(17), 6262-8.
159. Rapid multiserotype detection of human rhinoviruses on optically coated silicon surfaces.
160. Rachel Ostroff, Anna Ettinger, Helen La, Marynette Rihanek, Leora Zalman, James Meador III, Amy K. Patick, Steve Worland, Barry Polisky. Journal of Clinical Virology 21 (2001) 105-117
161. Inhibition of 3C protease from human rhinovirus strain 1B by peptidyl bromomethylketonehydrazides. Kati, Warren M.; Sham, Hing L.; McCall, J. Owen; Montgomery, Debra A.; Wang, Gary T.; Rosenbrook, William; Miesbauer, Laura; Buko, Alex; Norbeck, Daniel W. Discovery Research, Abbott Laboratories, Abbott Park, Ill., USA. Arch. Biochem. Biophys. (1999), 362(2), 363-375.
162. Small peptidic aldehyde inhibitors of human rhinovirus 3C protease. Shepherd, Timothy A.; Cox, Gregory A.; McKinney, Emma; Tang, Joseph; Wakulchik, Mark; Zimmerman, Ronald E.; Villarreal, Elcira C. Lilly Res. Lab., Eli Lilly Co., Indianapolis, Ind., USA. Bioorg. Med. Chem. Lett. (1996), 6(23), 2893-2896.
163. Ventoso I, Barco A, Carrasco L. Genetic selection of poliovirus 2Apro-binding peptides. J Virol. 1999 January; 73(1):814-8
164. Svitkin Y V, Gradi A, Imataka H, Morino S, Sonenberg N. Eukaryotic initiation factor 4GII (eIF4GII), but not eIF4GI, cleavage correlates with inhibition of host cell protein synthesis after human rhinovirus infection. J Virol. 1999 April; 73(4):3467-72.
165. Zamora M, Marissen W E, Lloyd R E. Multiple eIF4GI-specific protease activities present in uninfected and poliovirus-infected cells. J Virol. 2002 January; 76(1):165-77.
166. Lee C K, Wimmer E. Proteolytic processing of poliovirus polyprotein: elimination of 2Apro-mediated, alternative cleavage of polypeptide 3CD by in vitro mutagenesis. Virology. 1988 October; 166(2):405-14.
167. King, A. M. Q., Brown, F., Christian, P. & 8 other authors (2000). Picornaviruses. In Virus Taxonomy. Seventh Report of the International Committee for the Taxonomy of Viruses, pp. 657-673. Edited by M. H. V. Van Regenmortel, C. M. Fauquet, D. H. L. Bishop, C. H. Calisher, E. B. Carsten, M. K. Estes, S. M. Lemon, J. Maniloff, M. A. Mayo, D. J. McGeoch, C. R. Pringle & R. B. Wickner. San Diego: Academic Press.
168. Stanway, G., Hughes, P. J., Mountford, R. C., Minor, P. D. & Almond, J. W. (1984). The complete nucleotide sequence of a common cold virus: human rhinovirus 14. Nucleic Acids Res 12, 7859-7875.
169. Savolainen, C., Blomqvist, S., Mulders, M. N. & Hovi, T. (2002b). Genetic clustering of all 102 human rhinovirus prototype strains: serotype 87 is close to human enterovirus 70. J Gen Virol 83, 333-340.
170. Hellen C U, Lee C K, Wimmer E. Determinants of substrate recognition by poliovirus 2A proteinase. J Virol. 1992 June; 66(6):3330-8.
171. Ventoso I, Barco A, Carrasco L. Genetic selection of poliovirus 2Apro-binding peptides. J Virol. 1999 January; 73(1):814-8
172. Berger, A., and I. Schechter. 1970. Mapping the active site of papain with the aid of peptide substrates and inhibitors. Phil. Trans. R. Soc. Lond. B 257: 249-264.
173. Jewell J E, Ball L A, Rueckert R. Limited expression of poliovirus by vaccinia virus recombinants due to inhibition of the vector by proteinase 2A. J Virol. 1990 March; 64(3):1388-93.
174. Wang Q M, Johnson R B, Cox G A, Villarreal E C, Churgay L M, Hale J E.
175. Enzymatic characterization of refolded human rhinovirus type 14 2A protease expressed in Escherichia coli. J Virol. 1998 February; 72(2):1683-7.
176. http://www.cdc.gov/ncidod/dvrd/entrvirs.htm.
177. Rotbart H A. Meningitis and encephalitis. In: Rotbart H A, editor. Human enterovirus infections. Washington, D.C.: American Society for Microbiology Press; 1995 p. 271-89.
178. Rotbart H A: Enteroviral infections of the central nervous system. Clin Infect Dis 1995, 20:971-981.
179. Attia J. Hatala R, Cook D J, Wong J G: Does This Adult Subject Have Acute Meningitis? JAMA. 1999; 282(2) 175-181.

1. An isolated peptide comprising an amino acid sequence selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 and 47, said amino acid sequence being no more than 14 amino acids in length.

2. A composition comprising a substrate of a viral protease attached to at least one detactable moiety, said substrate comprising the amino acid sequence of claim 1.

3. The composition of claim 2, wherein said at least one detectable moiety is a pre-enzyme, and whereas cleavage of said substrate activates said pre-enzyme.

4. The composition of claim 2, wherein said at least one detectable moiety is a FRET pair, and whereas cleavage of said substrate generates a signal from said FRET pair.

5. The composition of claim 4, wherein the composition further comprising a separating moiety.

6. A composition being of the general formula:
X-Y-Z
wherein:
Y comprises a substrate of a viral protease said substrate comprising the amino acid sequence of claim 1, cleavage of X-Y-Z by said viral protease forming cleavage products X-Y′ and Y″-Z wherein Y′ is a first cleavage product of Y and Y″ is a second cleavage product of Y;
X comprises a detectable moiety; and
Z comprises a separating moiety capable of binding to a separate phase of a two phase separating system;
wherein said X-Y-2 does not form a contiguous portion of a natural substrate said viral protease.

7. (canceled)

8. The composition of claim 6, wherein said separating moiety Z is selected from the group consisting of an immunological binding agent, a magnetic binding moiety, a peptide binding moiety, an affinity binding moiety, a nucleic acid moiety.

9. A composition being of the general formula:
X-Y-Z
wherein:
Y comprises a substrate of a viral protease said substrate comprising the amino acid sequence of claim 1, cleavage of X-Y-Z by said viral protease forming cleavage products X-Y′ and Y″-Z wherein Y′ is a fast cleavage product of Y and Y″ is a second cleavage product of Y;
X or Z comprises a marker, either a detectable moiety and/or a separating moiety capable of separating between cleaved and uncleaved composition in a suited manner.
wherein said X-Y-Z does not form a contiguous portion of a natural substrate said viral protease.

10. (canceled)

11. A method for detecting at least one virus in a sample, the method comprising:
(a) contacting the sample with at least one of the compositions of claim 2, under conditions allowing cleavage of said substrate; and
(b) monitoring cleavage of said substrate, wherein said cleavage of said substrate is indicative of the presence of said at least one virus in said sample.

12.-16. (canceled)

17. A diagnostic kit for detection of at least one virus in a sample, the kit comprising at least one composition of claim 2 and reagents for detecting cleavage of said substrate.

18. A diagnostic kit comprising a packaging material and a plurality compositions for detecting presence of a plurality of viruses, wherein each of said compositions is of a general formula,
X-Y-Z
wherein:
Y comprises a substrate of a viral protease, cleavage of X-Y-Z by said viral protease forming cleavage products X-Y′ and Y″-Z wherein Y′ is a first cleavage product of Y and Y″ is a second cleavage product of Y;
X or Z comprises a marker, either a detectable moiety and/or a separating moiety capable of separating between cleaved and uncleaved compositions in a suited manner;
wherein said X-Y-Z does not form a contiguous portion of a natural substrate said viral protease,
wherein each of said X or Z comprise of at least one distinctively detectable moiety and whereas said packaging material comprises a label or package insert indicating that the kit is for detection of plurality of viruses in a sample.

19. A diagnostic kit comprising a packaging material and a plurality of compositions for detecting presence of a plurality of viruses, wherein each of said compositions is of a general formula,
X-Y-Z
wherein:
Y comprises a substrate of a viral protease, cleavage of X-Y-Z by said viral protease forming cleavage products X-Y′ and Y″-Z wherein Y′ is a first cleavage product of Y and Y″ is a second cleavage product of Y,
X comprises a detectable moiety; and
Z comprises a separating moiety capable of binding to a separate phase of a two phase separating system;
wherein said X-Y-Z does not form a contiguous portion of a natural substrate said viral protease,
wherein each of said X is distinctively detectable and whereas said packaging material comprises a label or package insert indicating that the kit is for detection of plurality of viruses in a sample.

20. The diagnostic kit of claim 18, wherein said plurality of compositions are attached to a single solid support.

21. The diagnostic kit of claim 20 wherein said distinctive detection is effected by an addressable location on said single solid support.

22. The diagnostic kit of claim 20 wherein said distinctive detection is effected by different detectable moieties.

23.-25. (canceled)

26. The diagnostic kit of claim 17, is a respiratory kit comprising at least two viruses selected from group consisting of Corona Viruses, SARS, HMPV (Human Meta pnemo virus), Influenza A+B, Avian Influenza, Adeno virus, RSV (Respiratory Syncytial Virus), Rhino virus, Para influenza viruses.

27. (canceled)

28. The diagnostic kit of claim 17, is a gastro-intestinal kit comprising at least two viruses selected from group consisting of Rota virus, Adeno 40/41, Hepatitis A, Hepatitis C, Hepatitis E, caliciviruses and CMV (Cytomegalovims).

29. The diagnostic kit of claim 17, is a meningitis kit comprising at least two viruses selected from group consisting of Enteroviruses (1-80), West Nile virus, Herpes Simplex 1, 2, and 6.

30. (canceled)

31. The diagnostic kit of claim 17, is a sexually transmitted diseases kit comprising at least two viruses selected from group consisting of HIV strain, Herpes simplex 1, Herpes simplex 2, HSV-1, HSV-2, HPV (Human Papilloma Viruses), and HTLV-1.

32. The diagnostic kit of claim 17, is a Traveler's kit comprising at least two viruses selected from group consisting of Hepatitis A, Hepatitis B, Hepatitis C, HIV, Herpes Virus 1 and 2.

33. The diagnostic kit of claim 17, is a veterinarian kit comprising at least two viruses selected from group consisting of Rabies and Distemper.

34. (canceled)

35. The diagnostic kit of claim 17, wherein said at least one virus comprises a plurality of viruses.

36.-38. (canceled)

39. The method or lets of claim 17, wherein said virus is HIV and said substrate comprises SEQ ID NO: 5.

40.-44. (canceled)

45. The kit of claim 17, wherein said virus is CMV virus and said substrate comprises SEQ ID NO: 14 or 15.

46.-48. (canceled)

49. The kit of claim 17, wherein said virus is West Nile virus and said substrate comprises SEQ ID NO: 21, 22 or 23.

50.-52. (canceled)

53. The kit of claim 17, wherein said virus is Hepatitis C virus and said substrate comprises SEQ ID NO: 33, 34 or 35.

54. (canceled)

55. The method or kits of claim 17, wherein said virus is Hepatitis A virus and said substrate comprises SEQ ID NO: 37 or 38.

56. The kit of claim 17, wherein said virus is HRV and said substrate comprises SEQ ID NO: 39 or 40.

57. The kit of claim 17, wherein said virus is Enterovirus and said substrate comprises SEQ ID NO: 41, 42, 43, 44, 45, 46 or 47.

58. The kit of claim 17, wherein said virus is an HRV virus and said substrate comprises SEQ ID NOS: 143-147, 150-151.

59. A method for designing a kinetically optimal substrate for a protease of a virus, the method comprising:
(a) identifying in a plurality of cleavage sequences of a polyprotein of at least one strain of the virus, a cleavage sequence displaying most rapid cleavage kinetics by the protease, and
(b) identifying a family-wide consensus cleavage sequence displaying most rapid cleavage kinetics, said family-wide consensus cleavage sequence being useful for designing the kinetically optimal substrate for the protease of the virus.

60.-62. (canceled)

63. The method of claim 59, wherein said viral protease is selected from the group consisting of a serine protease, a metalloprotease, an aspartic protease, a cysteine protease, a 3C proteinase, PA transcriptase, adenine protease, 2A protease, chimotrypsin or a trypsin. For example: NS3, NS2, NS-pro cysteine protease, nsP2 cysteine protease, nsP23pro, C protein protease, SFV NS, HIV aspartic protease, nsp4 Arteriviruses protease, HCMV protease. NS2-3, NS3-4Ap protease, HTLV-1 PR.

64.-67. (canceled)

68. An isolated peptide comprising an amino acid sequence selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 and 47, said amino acid sequence being no more than 14 amino acids in length and comprises mimetics for inhibiting activity of a respective viral protease.

69. Use of the peptide of claim 68 for the manufacture of a medicament identified for treating viral infection.

70. A pharmaceutical composition comprising as an active ingredient the isolated peptide of claim 68.
